(19) United States
(12) Patent Application Publication
(10) Pub. No.: US 2026/0250408 A1
(43) Pub. Date: Aug. 27, 2026

(54) COMPOSITIONS AND METHODS FOR TREATING MULTIPLE SCLEROSIS

(71) Applicants: Genentech, Inc., South San Francisco, CA (US); Hoffmann-La Roche Inc., Little Falls, NJ (US)

(72) Inventors: Heidemarie KLETZL, Basel (CH); Nina Jingzhu XIAO, South San Francisco, CA (US)

(21) Appl. No.: 19/667,436

(22) Filed: May 4, 2026

Related U.S. Application Data
(63) Continuation of application No. 19/444,781, filed on Jan. 9, 2026.
Application No. 19/444,781 is a continuation of application No. PCT/US2024/037434, filed on Jul. 10, 2024.
(60) Provisional application No. 63/513,109, filed on Jul. 11, 2023.
(60) Provisional application No. 63/588,938, filed on Oct. 9, 2023.

(51) Int. Cl. C07K 16/28; A61K 47/12; A61K 47/20; A61K 47/26; A61K 47/42; A61P 37/06

(52) U.S. Cl. CPC C07K 16/2887; A61K 47/12; A61K 47/20; A61K 47/26; A61K 47/42; A61P 37/06; C07K 2317/24

(57) ABSTRACT

The present invention relates to methods for treating multiple sclerosis (MS) in a patient which in some cases involves subcutaneously administering an anti-CD20 antibody into the patient at a dose of about 920 mg. Compositions, formulations and articles of manufacture with instructions for such use are also included.

### CROSS REFERENCE TO RELATED APPLICATION

This application is a continuation of U.S. application Ser. No. 19/444,781, filed on Jan. 9, 2026, which is a continuation of International Application No. PCT/US2024/037434, filed on Jul. 10, 2024, which claims the benefit of U.S. provisional application No. 63/513,109, filed Jul. 11, 2023, and U.S. provisional application No. 63/588,938, filed Oct. 9, 2023, the disclosures of which are herein incorporated by reference in their entirety.

### REFERENCE TO AN ELECTRONIC SEQUENCE LISTING

The content of the electronic sequence listing (146392067102SeqList.xml; Size: 11,885 bytes; and Date of Creation: May 1, 2026) is herein incorporated by reference in its entirety.

### FIELD

The present disclosure relates to methods for treating multiple sclerosis (MS) in a patient, and articles of manufacture with instructions for such use.

### BACKGROUND

Multiple sclerosis (MS) is a chronic, inflammatory, demyelinating, and degenerative disease of the central nervous system (CNS) that affects approximately 900,000 people in the United States (Wallin et al. (2019) Neurology. 92: e1029-40) and 2.3 million worldwide (GBD 2016 Multiple Sclerosis Collaborators. (2019) Lancet. 18:269-85). It is primarily a disease that first presents in young adults, with 70%-80% of patients having an age of onset (i.e., initial clinical presentation to a physician) between 20 and 40 years (Anderson et al. (1992) Ann Neurol 31:333-336; Noonan et al. (2002) Neurology. 58:136-138.) and has a gender bias influenced by the phenotype, with 64%-70% of diagnosed patients being women.

MS is classified into three clinical phenotypes: relapsing remitting (RRMS), secondary progressive (SPMS), and primary progressive (PPMS) (Lublin et al. (2014) Neurology. 83:278-86). These three phenotypes are further subdivided into active and non-active forms based on the presence or absence of disease activity, defined by the presence of clinical relapses and/or so-called active lesions on a magnetic resonance imaging (MRI) scan. Active MRI lesions are gadolinium-enhancing lesions on T1-weighted scan (T1Gd⁺) or new T2-weighted lesions/enlarging T2-weighted lesions. Relapsing MS (RMS) forms encompass RRMS and active SPMS, and progressive MS (PMS) forms constitute non-active SPMS and PPMS.

Evidence available to date suggests that despite the potential heterogeneity of the clinical expression of the disease, PPMS, SPMS, and RRMS belong to the same disease spectrum, and that pathological mechanisms responsible for relapses/disease activity and progression biology are largely identical across the MS spectrum (Lassmann (2018) Cold Spring Harb Perspect Med. 8 (3): a028936). Although the mechanisms associated with disease progression are assumed to be present from the onset of the disease (Cree et al. (2019) Curr Opin Neurol. 32:365-77), clinical disability progression manifests often later in the course of a patient's disease most likely due to the degree of brain reserve of the patient. The symptomatic worsening associated with MS disability progression results in a slow, insidious loss of a patient's motor and sensory function, as well as cognitive decline and autonomic dysfunctions.

Disability progression across the spectrum of MS might occur as a result of two concurrent inflammatory mechanisms: acute inflammation and chronic compartmentalized inflammation.

Acute inflammation can be observed on an MRI scan (as T1Gd⁺lesions or new T2 lesions/enlarging T2 lesions) and clinically manifests as relapses, where it can also lead to step-wise increase of disability due to incomplete relapse recovery. Pathophysiologically, relapsing forms of MS (i.e., RMS) are associated with focal T-cell and B-cell invasion, with blood brain barrier leakage that give rise to classic active demyelinating plaques in the white matter. However, RMS also harbors signs of progression biology/chronic compartmentalized inflammation.

By contrast to these acute inflammatory processes, chronic compartmentalized inflammation is responsible for an increase in disability that occurs independently from relapses or radiological disease activity and is characterized by demyelination and axonal loss (progression biology). Progressive forms of MS (i.e., PMS) are associated with a chronic and slow accumulation of T cells and B cells in the connective tissue spaces of the brain, without leakage of the blood brain barrier. There is a typical formation of subpial-demyelinated lesions in the cerebral and cerebellar cortex, with slow expansion of pre-existing lesions in the white matter and diffuse chronic inflammation in the normal appearing white or gray matter.

Ocrelizumab® (“OCR”) is a recombinant humanized, monoclonal IgG1 antibody that selectively targets and depletes CD20-expressing B cells, while preserving the capacity of B-cell reconstitution and preexisting humoral immunity. CD20 is a B-cell surface molecule that is restricted in expression to pre-B cells and mature B cells but is not expressed earlier in the development of B cells. Ocrelizumab® administered by intravenous (IV) infusion at a dose of 600 mg every 6 months was approved by the U.S. Food and Drug Administration (FDA) in 2017 for the treatment of adult patients with MS. All references cited herein, including patent applications, patent publications, and UniProtKB/Swiss-Prot Accession numbers are herein incorporated by reference in their entirety, as if each individual reference were specifically and individually indicated to be incorporated by reference.

### BRIEF SUMMARY OF THE APPLICATION

The present application in one aspect provides a method of treating multiple sclerosis in a patient comprising subcutaneously administering an anti-CD20 antibody to the patient at a dose of about 920 mg, wherein the anti-CD20 antibody comprises a VH (or V_{H}) domain comprising the amino acid sequence set forth in SEQ ID NO: 8, a VL (or V_{L}) domain comprising the amino acid sequence set forth in SEQ ID NO: 7, and a human IgG1 constant region. In some embodiments, the patient has not been subjected to a prior intravenous administration of the anti-CD20 antibody at a dose of about 600 mg. In some embodiments, the patient has not been subjected to a prior intravenous administration of the anti-CD20 antibody. In some embodiments, the patient has not been subjected to a prior subcutaneous administration of the anti-CD20 antibody at a dose lower than about 900 mg. In some embodiments, the patient has received a prior treatment with an anti-CD20 antibody, optionally wherein the prior treatment comprises an intravenous administration of the anti-CD20 antibody, optionally the prior treatment comprises the intravenous administration of the anti-CD20 antibody at a dose of about 300 mg or about 600 mg.

In some embodiments according to any of the methods described above or herein, the method comprises subcutaneously administering more than one dose of the anti-CD20 antibody, wherein each dose comprises about 920 mg of the anti-CD20 antibody. In some embodiments, the method comprises administering at least two, three or four doses of the antibody anti-CD20 antibody.

In some embodiments according to any of the methods described above or herein, the anti-CD20 antibody is administered at a frequency of no more than once every 24 weeks or about once every 24 weeks.

In some embodiments according to any of the methods described above or herein, the anti-CD20 antibody is administered at a frequency of no more than once every 6 months or about once every 6 months.

In some embodiments according to any of the methods described above or herein, the anti-CD20 antibody is the only medicament administered to the patient to treat multiple sclerosis.

In some embodiments according to any of the methods described above or herein, the anti-CD20 antibody is in a pharmaceutical formulation, wherein the pharmaceutical formulation comprises: a. about 35-45 mg/ml (e.g., 40 mg/ml) of anti-CD20 antibody; b. about 20 mM sodium acetate providing a pH of about 5 to about 5.6 (e.g., 5.3); c. about 190 mM to about 290 mM (e.g., 240 mM) trehalose; d. about 5 mM to 15 mM (e.g., 10 mM) methionine; e. about 0.04-0.08% (w/v) (e.g., 0.06%) polysorbate 20; and f. about 1000 U/ml hyaluronidase. In some embodiments according to any of the methods described above or herein, the anti-CD20 antibody is in a pharmaceutical formulation, wherein the pharmaceutical formulation comprises: a. about 40 mg/ml of anti-CD20 antibody; b. about 20 mM sodium acetate providing a pH of about 5.3; c. about 240 mM trehalose; d. about 10 mM methionine; e. about 0.06% (w/v) polysorbate 20; and f. about 1000 U/ml hyaluronidase.

In some embodiments according to any of the methods described above or herein, the patient has been subject to oral dexamethasone (or an equivalent corticosteroid) and antihistamine shortly prior to the subcutaneous administration of the anti-CD20 antibody, optionally the oral dexamethasone (or an equivalent corticosteroid) and antihistamine are administered within 30 minutes, 20 minutes, or 15 minutes prior to the subcutaneous administration.

In some embodiments according to any of the methods described above or herein, the anti-CD20 antibody comprises a light chain comprising the amino acid sequence of SEQ ID NO: 9 and a heavy chain comprising the amino acid sequence of SEQ ID NO: 10 or 11.

In some embodiments according to any of the methods described above or herein, the anti-CD20 antibody is ocrelizumab.

In some embodiments according to any of the methods described above or herein, the multiple sclerosis is relapsing multiple sclerosis (RMS). In some embodiments, the patient has a clinical isolated syndrome (CIS), relapsing-remitting multiple sclerosis (RRMS) or active secondary progressive multiple sclerosis (SPMS).

In some embodiments according to any of the methods described above or herein, the multiple sclerosis is primary progressive multiple sclerosis (PPMS).

In some embodiments according to any of the methods described above or herein, the treatment achieves a) the result of being non-inferior to an intravenous infusion of said anti-CD20 antibody (e.g., at a dose of 600 mg), as measured by pharmacokinetics (levels in the blood, e.g., serum area under the curve (AUC)) over 12 weeks, b) the result of having comparable MRI lesion activity in the brain of the patient over 12 weeks to an intravenous infusion of said anti-CD20 antibody (e.g., at a dose of 600 mg), and/or c) a safety profile consistent with that of an intravenous infusion of said anti-CD20 antibody (e.g., at a dose of 600 mg).

In some embodiments according to any of the methods described above or herein, the anti-CD20 antibody is comprised in a liquid formulation, and the subcutaneous administration takes no more than 10 minutes.

The present application in another aspect provides a liquid formulation comprising: a. about 35 to about 45 mg/mL (e.g., 40 mg/ml) anti-CD20 antibody; b. about 20 mM sodium acetate providing a pH of about 5.0 to about 5.6 (e.g., a pH of 5.3); c. about 190 to about 290 mM (e.g., 240 mM) trehalose; d. about 5 to about 15 mM (e.g., 10 mM) methionine; e. about 0.04-0.08% (w/v) (e.g., 0.06% (w/v)) polysorbate 20; and f. about 1000 U/ml hyaluronidase, wherein the anti-CD20 antibody comprises a VH domain comprising the amino acid sequence set forth in SEQ ID NO: 8, a VL domain comprising the amino acid sequence set forth in SEQ ID NO: 7, and a human IgG1 constant region. In some embodiments, the anti-CD20 antibody is ocrelizumab. In some embodiments, the formulation is for subcutaneous administration.

The present application in another aspect provides a liquid formulation comprising: a. about 40 mg/ml anti-CD20 antibody; b. about 20 mM sodium acetate providing a pH of about 5.3; c. about 240 mM trehalose; d. about 10 mM methionine; e. about 0.06% (w/v) polysorbate 20; and f. about 1000 U/ml hyaluronidase, wherein the anti-CD20 antibody comprises a VH domain comprising the amino acid sequence set forth in SEQ ID NO: 8, a VL domain comprising the amino acid sequence set forth in SEQ ID NO: 7, and a human IgG1 constant region. In some embodiments, the anti-CD20 antibody is ocrelizumab. In some embodiments, the formulation is for subcutaneous administration.

The present application in another aspect provides a unit dosage form of an anti-CD20 antibody comprising a sealed vial containing a quantity of an anti-CD20 antibody suitable for subcutaneous administration to a patient, wherein said quantity is sufficient to deliver a dose of about 920 mg, wherein the anti-CD20 antibody comprises a VH domain comprising the amino acid sequence set forth in SEQ ID NO: 8, a VL domain comprising the amino acid sequence set forth in SEQ ID NO: 7, and a human IgG1 constant region. In some embodiments, the anti-CD20 antibody is comprised in a liquid formulation at the concentration of about 35-45 mg/ml (e.g., 40 mg/ml). In some embodiments, said formulation further comprises a buffering agent, a stabilizer and surfactant, optionally wherein the formulation further comprises a hyaluronidase. In some embodiments, the buffering agent comprises sodium acetate, optionally wherein the formulation comprises about 20 mM sodium acetate providing a pH of about 5 to about 5.6 (e.g., 5.3). In some embodiments, the stabilizer comprises trehalose and/or methionine, optionally wherein the formulation comprises a) about 190 mM to about 290 mM (e.g., 240 mM) trehalose and b) about 5 mM to about 15 mM (e.g., 10 mM) methionine. In some embodiments, the surfactant comprises polysorbate 20, and optionally wherein the formulation comprises about 0.04-0.08% (w/v) (e.g., 0.06% (w/v)) polysorbate 20. In some embodiments, the hyaluronidase comprises a recombinant human hyaluronidase, optionally the recombinant human hyaluronidase is rHuPH20, further optionally the formulation comprises about 1000 U/ml hyaluronidase. In some embodiments, the formulation comprises about 23,000 units of hyaluronidase in a unit dose.

The present application in another aspect provides an article of manufacture containing a single fixed dose of an anti-CD20 antibody comprising a VH domain comprising the amino acid sequence set forth in SEQ ID NO: 8, a VL domain comprising the amino acid sequence set forth in SEQ ID NO: 7, and a human IgG1 constant region, wherein the fixed dose is about 920 mg. In some embodiments, the article of manufacture comprises a single-used vial. In some embodiments, the article of manufacture comprises a syringe or a device comprising a needle, optionally wherein the device is an on-body device. In some embodiments, the anti-CD20 antibody is ocrelizumab. In some embodiments, the article of manufacture further comprises a package insert instructing a user to administer the fixed doses of the anti-CD20 antibody subcutaneously to a patient with multiple sclerosis, optionally wherein the user is the patient.

The present application in another aspect provides a single-use vial containing a quantity of an anti-CD20 antibody suitable for subcutaneous administration to a patient, wherein said quantity is sufficient to deliver a dose of about 920 mg, wherein the anti-CD20 antibody comprises a VH domain comprising the amino acid sequence set forth in SEQ ID NO: 8, a VL domain comprising the amino acid sequence set forth in SEQ ID NO: 7, and a human IgG1 constant region. In some embodiments, the anti-CD20 antibody is ocrelizumab.

### BRIEF DESCRIPTION OF THE DRAWINGS

The drawings illustrate certain features and advantages of this disclosure. These embodiments are not intended to limit the scope of the appended claims in any manner.

FIG. 1 provides a schematic for a Phase III, non-inferiority, randomized, open-label, parallel group, multicenter study to investigate the pharmacokinetics, pharmacodynamics, safety and radiological and clinical effects of subcutaneous (SC) ocrelizumab in multiple sclerosis.

FIG. 2A depicts pharmacokinetics results of the OCARINA II study.

FIG. 2B depict pharmacokinetics results of the OCARINA II study.

FIG. 3A depicts radiological and clinical effects of the OCARINA II study.

FIG. 3B depicts radiological and clinical effects of the OCARINA II study.

FIG. 3C depicts relapses observed in the OCARINA II study.

FIG. 4 depicts B-cell depletion results of the OCARINA II study.

FIG. 5 depicts B-cell depletion results of the OCARINA I study.

### DETAILED DESCRIPTION

The present application is at least partly based upon Applicant's findings that administering an anti-CD20 antibody (e.g., ocrelizumab) subcutaneously (SC) at a dose of about 920 mg, optionally once every six months or 24 weeks for at least 12, 18, or 24 months, to an individual having multiple sclerosis (MS) can achieve non-inferior exposure and/or similar advantageous therapeutic effects demonstrated by the currently approved intravenous (IV) administration of ocrelizumab at a dose of 600 mg. In a phase III clinical trial, ocrelizumab subcutaneous injection (e.g., at a dose of about 920 mg) was shown to be non-inferior to ocrelizumab given by intravenous infusion (IV) (e.g., at a dose of about 600 mg), as measured by pharmacokinetics (levels in the blood) over 12 weeks. Ocrelizumab subcutaneous injection (e.g., at a dose of about 920 mg) was also comparable with ocrelizumab given by intravenous infusion (IV) (e.g., at a dose of about 600 mg) in controlling magnetic resonance imaging (MRI) lesion activity in the brain over 12 weeks. The safety profile of ocrelizumab subcutaneous injection (e.g., at a dose of about 920 mg) was consistent with that of ocrelizumab given by intravenous infusion (IV) (e.g., at a dose of about 600 mg).

The present method has various benefits over previous methods of multiple sclerosis treatment such as IV administration of ocrelizumab at a dose of 600 mg. These benefits include more convenient and faster administration times, fewer administrations, enabling administration at locations which lack additional infusion capacity or are absent of structure and experience for IV infusion, and the potential for administration outside of a controlled healthcare setting such as home administration.

In some aspects, the present application provides methods of treating multiple sclerosis in a patient comprising subcutaneously administering an anti-CD20 antibody to the patient every six months or 24 weeks at a dose of about 920 mg, wherein the anti-CD20 antibody is ocrelizumab. In some embodiments, the present application provides methods of treating multiple sclerosis in a patient comprising subcutaneously administering an anti-CD20 antibody (e.g., ocrelizumab) every six months or 24 weeks at a dose of about 920 mg, achieving one or more of the following: a) the result of being non-inferior to an intravenous infusion of said anti-CD20 antibody (e.g., at a dose of 600 mg), as measured by pharmacokinetics (levels in the blood, e.g., serum area under the curve (AUC)) over 12 weeks; b) the result of having comparable magnetic resonance imaging (MRI) lesion activity in the brain of the patient over 12 weeks to an intravenous infusion of said anti-CD20 antibody (e.g., at a dose of 600 mg); and/or c) a safety profile consistent with that of an intravenous infusion of said anti-CD20 antibody (e.g., at a dose of 600 mg). In some embodiments, the anti-CD20 antibody is ocrelizumab.

### I. Definitions

A “B cell” is a lymphocyte that matures within the bone marrow, and includes a naïve B cell, memory B cell, or effector B cell (plasma cells). The B cell herein may be a normal or non-malignant B cell.

A “B-cell surface marker” or “B-cell surface antigen” herein is an antigen expressed on the surface of a B cell that can be targeted with an antibody that binds thereto. Exemplary B-cell surface markers include the CD10, CD19, CD20, CD21, CD22, CD23, CD24, CD37, CD40, CD53, CD72, CD73, CD74, CDw75, CDw76, CD77, CDw78, CD79a, CD79b, CD80, CD81, CD82, CD83, CDw84, CD85 and CD86 leukocyte surface markers (for descriptions, see The Leukocyte Antigen Facts Book, 2nd Edition. 1997, ed. Barclay et al. Academic Press, Harcourt Brace & Co., New York). Other B-cell surface markers include RP105, FcRH2, B-cell CR2, CCR6, P2X5, HLA-DOB, CXCR5, FCER2, BR3, Btig, NAG14, SLGC16270, FcRH1, IRTA2, ATWD578, FcRH3, IRTA1, FcRH6, BCMA, and 239287. The B-cell surface marker of particular interest herein is preferentially expressed on B cells compared to other non-B-cell tissues of a mammal and may be expressed on both precursor B cells and mature B cells. The preferred B-cell surface marker herein is CD20.

The “CD20” antigen, or “CD20,” is an about 35-kDa, non-glycosylated phosphoprotein found on the surface of greater than 90% of B cells from peripheral blood or lymphoid organs. CD20 is present on both normal B cells as well as malignant B cells, but is not expressed on stem cells. Other names for CD20 in the literature include “B-lymphocyte-restricted antigen” and “Bp35”. The CD20 antigen is described in Clark et al. Proc. Natl. Acad. Sci. (USA) 82:1766 (1985), for example.

“Antibodies” are usually heterotetrameric glycoproteins of about 150,000 daltons, composed of two identical light (L) chains and two identical heavy (H) chains. Each light chain is linked to a heavy chain by one covalent disulfide bond, while the number of disulfide linkages varies among the heavy chains of different immunoglobulin isotypes. Each heavy and light chain also has regularly spaced intrachain disulfide bridges. Each heavy chain has at one end a variable domain (VH or V_{H}) followed by a number of constant domains. Each light chain has a variable domain at one end (VL or V_{L}) and a constant domain at its other end; the constant domain of the light chain is aligned with the first constant domain of the heavy chain, and the light chain variable domain is aligned with the variable domain of the heavy chain. Particular amino acid residues are believed to form an interface between the light chain and heavy chain variable domains.

The “light chains” of antibodies (immunoglobulins) from mammalian species can be assigned to one of two clearly distinct types, called kappa (κ) and lambda (2), based on the amino acid sequences of their constant domains.

The “heavy chains” of antibodies from mammalian species can also be assigned to different classes. There are five major classes of intact antibodies: IgA, IgD, IgE, IgG, and IgM, and several of these may be further divided into subclasses (isotypes), e.g., IgG1, IgG2, IgG3, IgG4, IgA, and IgA2. The heavy chain constant domains that correspond to the different classes of antibodies are called a, 8, &, Y, and u, respectively. The subunit structures and three-dimensional configurations of different classes of immunoglobulins are well known.

The term “ocrelizumab” “OCR” or “Ocrevus®” (CAS Registration No. 637334-45-3) herein refers to the genetically engineered humanized monoclonal antibody directed against the CD20 antigen and comprising (a) a light chain comprising the amino acid sequence of SEQ ID NO: 9 and (b) a heavy chain comprising the amino acid sequence of SEQ ID NO: 10 or 11. Ocrelizumab is available from Genentech.

A “subject” or “patient” herein is a human subject or patient. Generally, the subject or patient is eligible for treatment for multiple sclerosis. For the purposes herein, such eligible subject or patient is one who is experiencing, has experienced, or is likely to experience, one or more signs, symptoms or other indicators of multiple sclerosis; has been diagnosed with multiple sclerosis, whether, for example, newly diagnosed (with “new onset” MS), previously diagnosed with a new relapse or exacerbation, previously diagnosed and in remission, etc.; and/or is at risk for developing multiple sclerosis. One suffering from or at risk for suffering from multiple sclerosis may optionally be identified as one who has been screened for elevated levels of CD20-positive B cells in serum, cerebrospinal fluid (CSF) and/or MS lesion(s) and/or is screened for using an assay to detect autoantibodies, assessed qualitatively, and preferably quantitatively. Exemplary such autoantibodies associated with multiple sclerosis include anti-myelin basic protein (MBP), anti-myelin oligodendrocytic glycoprotein (MOG), anti-ganglioside and/or anti-neurofilament antibodies. Such autoantibodies may be detected in the subject's serum, cerebrospinal fluid (CSF) and/or MS lesion. By “elevated” autoantibody or B cell level(s) herein is meant level(s) of such autoantibodies or B cells which significantly exceed the level(s) in an individual without MS.

As used herein, “treatment” or “treating” is an approach for obtaining beneficial or desired results including clinical results. For purposes of this invention, beneficial or desired clinical results include, but are not limited to, one or more of the following: decreasing one or more symptoms resulting from the disease, diminishing the extent of the disease, stabilizing the disease (e.g., preventing or delaying the worsening of the disease), delay or slowing the progression of the disease, ameliorating the disease state, decreasing the dose of one or more other medications required to treat the disease, and/or increasing the quality of life.

As used herein, “delaying” or “slowing” the progression of multiple sclerosis means to prevent, defer, hinder, slow, retard, stabilize, and/or postpone development of the disease. This delay can be of varying lengths of time, depending on the history of the disease and/or individual being treated.

A “symptom” of MS is any morbid phenomenon or departure from the normal in structure, function, or sensation, experienced by the subject and indicative of MS.

“Multiple sclerosis” refers to the chronic inflammatory, often disabling disease of the central nervous system characterized by demyelination and neurodegeneration. There are three internationally recognized forms of MS, namely, primary progressive multiple sclerosis (PPMS), relapsing-remitting multiple sclerosis (RRMS), and secondary progressive multiple sclerosis (SPMS).

“Progressive multiple sclerosis” as used herein refers to primary progressive multiple sclerosis (PPMS), and secondary progressive multiple sclerosis (SPMS). In some embodiments, progressive multiple sclerosis is characterized by documented, irreversible loss of neurological function persisting for ≥6 months that cannot be attributed to clinical relapse.

“Primary progressive multiple sclerosis” or “PPMS” is characterized by a gradual progression of the disease from its onset with rare superimposed relapses and remissions. There may be periods of a leveling off of disease activity and there may be good and bad days or weeks. PPMS differs from RRMS and SPMS in that onset is typically in the late thirties or early forties, men are as likely women to develop it, and initial disease activity is often in the spinal cord and not in the brain. PPMS disease activity can also be observed (or found) in the brain. PPMS is the sub-type of MS that is least likely to show inflammatory (gadolinium enhancing) lesions on MRI scans. The Primary Progressive form of the disease affects about 15% of all people with multiple sclerosis. PPMS may be defined according to the criteria in Thompson et al. (2018) Lancet 7 (2): 162-173. The subject with PPMS treated herein is usually one with probable or definitive diagnosis of PPMS.

“Relapsing-remitting multiple sclerosis” or “RRMS” is characterized by relapses (also known as exacerbations) during which time new symptoms can appear and old ones resurface or worsen. The relapses are followed by periods of remission, during which time the person fully or partially recovers from the deficits acquired during the relapse. Relapses can last for days, weeks or months and recovery can be slow and gradual or almost instantaneous. The vast majority (about 85%) of people presenting with MS are first diagnosed with RRMS. This is typically when they are in their twenties or thirties, though diagnoses much earlier or later are known. Twice as many women as men present with this sub-type of MS. During relapses, myelin, a protective insulating sheath around the nerve fibers (neurons) in the white matter regions of the central nervous system (CNS), may be damaged in an inflammatory response by the body's own immune system. This causes a wide variety of neurological symptoms that vary considerably depending on which areas of the CNS are damaged. Immediately after a relapse, the inflammatory response dies down and a special type of glial cell in the CNS (called an oligodendrocyte) sponsors remyelination—a process whereby the myelin sheath around the axon may be repaired. It is this remyelination that may be responsible for the remission. Approximately 50% of patients with RRMS convert to SPMS within 10 years of disease onset. After 30 years, this figure rises to 90%. At any one time, the relapsing-remitting form of the disease accounts around 55% of all people with MS.

In some embodiments, an initial or first “antibody dose” refers to contact with or exposure to the antibody herein in one or more infusions administered over a period of time of about 1-20 days. The infusions may be given at one time or at fixed or irregular time intervals over this period of exposure. Initial and later (e.g. second or third) antibody doses are separated in time from each other as described in detail herein. In some embodiments, an initial or first “antibody dose” refers to subcutaneous administration.

As used herein, an “interval” between antibody doses refers to time period between an earlier antibody dose and a later antibody dose.

A “stable” formulation is one in which the protein therein essentially retains its physical stability and/or chemical stability and/or biological activity upon storage. Preferably, the formulation essentially retains its physical and chemical stability, as well as its biological activity upon storage. The storage period is generally selected based on the intended shelf-life of the formulation. Various analytical techniques for measuring protein stability are available in the art and are reviewed in Peptide and Protein Drug Delivery, 247-301, Vincent Lee Ed., Marcel Dekker, Inc., New York, N.Y., Pubs. (1991) and Jones, A. Adv. Drug Delivery Rev. 10:29-90 (1993), for example. Stability can be measured at a selected temperature for a selected time period. Stability can be evaluated qualitatively and/or quantitatively in a variety of different ways, including evaluation of aggregate formation (for example using size exclusion chromatography, by measuring turbidity, and/or by visual inspection); by assessing charge heterogeneity using cation exchange chromatography, image capillary isoelectric focusing (icIEF) or capillary zone electrophoresis; amino-terminal or carboxy-terminal sequence analysis; mass spectrometric analysis; SDS-PAGE analysis to compare reduced and intact antibody; peptide map (for example tryptic or LYS-C) analysis; evaluating biological activity or antigen binding function of the antibody; etc. Instability may involve any one or more of: aggregation, deamidation (e.g., Asn deamidation), oxidation (e.g., Met oxidation), isomerization (e.g., Asp isomeriation), clipping/hydrolysis/fragmentation (e.g., hinge region fragmentation), succinimide formation, unpaired cysteine(s), N-terminal extension, C-terminal processing, glycosylation differences, etc.

A protein “retains its physical stability” in a pharmaceutical formulation if it shows no signs or very little of aggregation, precipitation and/or denaturation upon visual examination of color and/or clarity, or as measured by UV light scattering or by size exclusion chromatography.

A protein “retains its chemical stability” in a pharmaceutical formulation, if the chemical stability at a given time is such that the protein is considered to still retain its biological activity as defined below. Chemical stability can be assessed by detecting and quantifying chemically altered forms of the protein. Chemical alteration may involve size modification (e.g. clipping) which can be evaluated using size exclusion chromatography, SDS-PAGE and/or matrix-assisted laser desorption ionization/time-of-flight mass spectrometry (MALDI/TOF MS), for example. Other types of chemical alteration include charge alteration (e.g. occurring as a result of deamidation) which can be evaluated by ion-exchange chromatography or icIEF, for example.

An antibody “retains its biological activity” in a pharmaceutical formulation, if the biological activity of the antibody at a given time is at least about 60% (within the errors of the assay) of the biological activity exhibited at the time the pharmaceutical formulation was prepared as determined in an assay (e.g., an antigen binding assay). Other “biological activity” assays for antibodies are elaborated herein below.

As used herein, “biological activity” of a monoclonal antibody includes the ability of the antibody to bind to antigen and resulting in a measurable biological response which can be measured in vitro or in vivo.

“Corticosteroid” refers to any one of several synthetic or naturally occurring substances with the general chemical structure of steroids that mimic or augment the effects of the naturally occurring corticosteroids. Examples of synthetic corticosteroids include prednisone, prednisolone (including methylprednisolone), dexamethasone, glucocorticoid and betamethasone.

A “package insert” is used to refer to instructions customarily included in commercial packages of therapeutic products, that contain information about the indications, usage, dosage, administration, contraindications, other therapeutic products to be combined with the packaged product, and/or warnings relating to the use of such therapeutic products, etc.

A “label” is used herein to refer to information customarily included with commercial packages of pharmaceutical formulations including containers such as vials and package inserts, as well as other types of packaging.

Reference to “about” a value or parameter herein includes (and describes) variations that are directed to that value or parameter per se. For example, description referring to “about X” includes description of “X.” In some embodiments, “about” a value or parameter describes a range of 50% to 150%, 60% to 140%, 70% to 130%, 80% to 120%, 90% to 110%, or 95% to 105% of the value or parameter.

As used herein and in the appended claims, the singular forms “a,” “or,” and “the” include plural referents unless the context clearly dictates otherwise. It is understood that aspects and variations of the invention described herein include “consisting” and/or “consisting essentially of” aspects and variations.

It is to be understood that one, some, or all of the properties of the various embodiments described herein may be combined to form other embodiments of the present invention. These and other aspects of the invention are apparent to one of skill in the art.

All references cited herein, including patent applications and publications, are incorporated by reference in their entirety.

### II. Methods of Treatment

The various aspects and embodiments described in this section in the context of a method of treatment also apply to an anti-CD20 antibody for use according to the methods described herein. Similarly, the various aspects and embodiments described in this section in the context of a method of treatment also apply to an anti-CD20 antibody for use with a delivery device, according to the methods described herein, and an anti-CD20 antibody for use according to the methods described herein, in combination with a delivery device, and an anti-CD20 antibody for use according to the methods described herein, wherein the anti-CD20 antibody is administered by a delivery device (e.g., an on-body device).

In some embodiments, there is provided a method of treating multiple sclerosis in a patient comprising subcutaneously administering an anti-CD20 antibody to the patient at a dose of about 920 mg, wherein the anti-CD20 antibody comprises a V_{H} domain comprising the amino acid sequence set forth in SEQ ID NO: 8, a V_{L} domain comprising the amino acid sequence set forth in SEQ ID NO: 7, and a human IgG1 constant region, wherein the patient has not been subject to a prior administration of the anti-CD20 antibody. In some embodiments, the anti-CD20 antibody comprises a light chain comprising the amino acid sequence of SEQ ID NO: 9 and a heavy chain comprising the amino acid sequence of SEQ ID NO: 10 or 11. In some embodiments, the anti-CD20 antibody is ocrelizumab. In some embodiments, the multiple sclerosis is relapsing multiple sclerosis (RMS). In some embodiments, the patient has a clinical isolated syndrome (CIS), relapsing-remitting multiple sclerosis (RRMS) or active secondary progressive multiple sclerosis (SPMS). In some embodiments, the multiple sclerosis is a primary progressive multiple sclerosis (PPMS). In some embodiments, the patient has taken or been administered oral dexamethasone (or an equivalent corticosteroid) and/or antihistamine shortly prior to the subcutaneous administration of the anti-CD20 antibody, optionally the oral dexamethasone (or an equivalent corticosteroid) and antihistamine are administered within 30 minutes, 20 minutes, or 15 minutes prior to the subcutaneous administration. In some embodiments, the anti-CD20 antibody is the only medicament administered to the patient to treat multiple sclerosis. In some embodiments, the anti-CD20 antibody is comprised in a liquid formulation at the concentration of about 40 mg/ml. Said formulation further comprises a buffering agent, a stabilizer and surfactant, optionally wherein the formulation further comprises a hyaluronidase. In some embodiments, the buffering agent comprises sodium acetate, optionally wherein the formulation comprises about 20 mM sodium acetate providing a pH of about 5.3. In some embodiments, the stabilizer comprises trehalose and/or methionine, optionally wherein the formulation comprises a) about 240 mM trehalose and b) about 10 mM methionine. In some embodiments, the surfactant comprises polysorbate 20, and optionally wherein the formulation comprises about 0.06% (w/v) polysorbate 20. In some embodiments, the hyaluronidase comprises a recombinant human hyaluronidase, optionally the recombinant human hyaluronidase is rHuPH20, further optionally the formulation comprises about 1000 U/ml hyaluronidase. In some embodiments, the formulation comprises about 23,000 units of hyaluronidase. In some embodiments, the anti-CD20 antibody is comprised in a liquid formulation at the concentration of about 35-45 mg/ml (e.g., 40 mg/ml). In some embodiments, said formulation further comprises a buffering agent, a stabilizer and surfactant, optionally wherein the formulation further comprises a hyaluronidase. In some embodiments, the buffering agent comprises sodium acetate, optionally wherein the formulation comprises about 20 mM sodium acetate providing a pH of about 5 to about 5.6 (e.g., 5.3). In some embodiments, the stabilizer comprises trehalose and/or methionine, optionally wherein the formulation comprises a) about 190 mM to about 290 mM (e.g., 240 mM) trehalose and b) about 5 mM to about 15 mM (e.g., 10 mM) methionine. In some embodiments, the surfactant comprises polysorbate 20, and optionally wherein the formulation comprises about 0.04-0.08% (w/v) (e.g., 0.06% (w/v)) polysorbate 20. In some embodiments, the hyaluronidase comprises a recombinant human hyaluronidase, optionally the recombinant human hyaluronidase is rHuPH20, further optionally the formulation comprises about 1000 U/ml hyaluronidase. In some embodiments, the formulation comprises about 23,000 units of hyaluronidase. In some embodiments, the subcutaneous administration takes no more than 10 minutes. In some embodiments, the treatment achieves the result of being non-inferior to an intravenous infusion of said anti-CD20 antibody (e.g., at a dose of 600 mg), as measured by pharmacokinetics (levels in the blood, e.g., serum area under the curve (AUC)) over 12 weeks. In some embodiments, the treatment achieves the result of having comparable MRI lesion activity in the brain of the patient over 12 weeks to an intravenous infusion of said anti-CD20 antibody (e.g., at a dose of 600 mg). In some embodiments, the treatment achieves the result of a rapid, complete and sustained B-cell depletion. In some embodiments, the treatment achieves a safety profile consistent with that of an intravenous infusion of said anti-CD20 antibody (e.g., at a dose of 600 mg). In some embodiments, the anti-CD20 antibody is in a pharmaceutical formulation, wherein the pharmaceutical formulation comprises about 920 mg of the anti-CD20 antibody, about 23,000 units of hyaluronidase, about 2.088 g of α,α-trehalose dihydrate, about 5.5 mg of glacial acetic acid, about 34.4 mg of L-methionine, about 13.8 mg of polysorbate 20, about 50.1 mg of sodium acetate trihydrate, and water for injection at a pH of about 5.3. In some embodiments, the anti-CD20 antibody is ocrelizumab. In some embodiments, the anti-CD20 antibody is supplied in a 50 mL single-dose vial. In some embodiments, the formulation is in a volume of 23 mL.

In some embodiments, there is provided a method of treating multiple sclerosis in a patient comprising subcutaneously administering an anti-CD20 antibody to the patient at a dose of about 920 mg, wherein the anti-CD20 antibody comprises a V_{H} domain comprising the amino acid sequence set forth in SEQ ID NO: 8, a V_{L} domain comprising the amino acid sequence set forth in SEQ ID NO: 7, and a human IgG1 constant region, wherein the patient has not been subject to a prior administration of the anti-CD20 antibody (e.g., at a dose of about 600 mg). In some embodiments, the anti-CD20 antibody comprises a light chain comprising the amino acid sequence of SEQ ID NO: 9 and a heavy chain comprising the amino acid sequence of SEQ ID NO: 10 or 11. In some embodiments, the anti-CD20 antibody is ocrelizumab. In some embodiments, the multiple sclerosis is relapsing multiple sclerosis (RMS). In some embodiments, the patient has a clinical isolated syndrome (CIS), relapsing-remitting multiple sclerosis (RRMS) or active secondary progressive multiple sclerosis (SPMS). In some embodiments, the multiple sclerosis is a primary progressive multiple sclerosis (PPMS). In some embodiments, the patient has been subject to oral dexamethasone (or an equivalent corticosteroid) and antihistamine shortly prior to the subcutaneous administration of the anti-CD20 antibody, optionally the oral dexamethasone (or the equivalent corticosteroid) and antihistamine are administered within 30 minutes, 20 minutes, or 15 minutes prior to the subcutaneous administration. In some embodiments, the anti-CD20 antibody is the only medicament administered to the patient to treat multiple sclerosis. In some embodiments, the anti-CD20 antibody is comprised in a liquid formulation at the concentration of about 40 mg/ml. said formulation further comprises a buffering agent, a stabilizer and surfactant, optionally wherein the formulation further comprises a hyaluronidase. In some embodiments, the buffering agent comprises sodium acetate, optionally wherein the formulation comprises about 20 mM sodium acetate providing a pH of about 5.3. In some embodiments, the stabilizer comprises trehalose and/or methionine, optionally wherein the formulation comprises a) about 240 mM trehalose and b) about 10 mM methionine. In some embodiments, the surfactant comprises polysorbate 20, and optionally wherein the formulation comprises about 0.06% (w/v) polysorbate 20. In some embodiments, the hyaluronidase comprises a recombinant human hyaluronidase, optionally the recombinant human hyaluronidase is rHuPH20, further optionally the formulation comprises about 1000 U/ml hyaluronidase. In some embodiments, the formulation comprises about 23,000 units of hyaluronidase. In some embodiments, the anti-CD20 antibody is comprised in a liquid formulation at the concentration of about 35-45 mg/ml (e.g., 40 mg/ml). In some embodiments, said formulation further comprises a buffering agent, a stabilizer and surfactant, optionally wherein the formulation further comprises a hyaluronidase. In some embodiments, the buffering agent comprises sodium acetate, optionally wherein the formulation comprises about 20 mM sodium acetate providing a pH of about 5 to about 5.6 (e.g., 5.3). In some embodiments, the stabilizer comprises trehalose and/or methionine, optionally wherein the formulation comprises a) about 190 mM to about 290 mM (e.g., 240 mM) trehalose and b) about 5 mM to about 15 mM (e.g., 10 mM) methionine. In some embodiments, the surfactant comprises polysorbate 20, and optionally wherein the formulation comprises about 0.04-0.08% (w/v) (e.g., 0.06% (w/v)) polysorbate 20. In some embodiments, the hyaluronidase comprises a recombinant human hyaluronidase, optionally the recombinant human hyaluronidase is rHuPH20, further optionally the formulation comprises about 1000 U/ml hyaluronidase. In some embodiments, the formulation comprises about 23,000 units of hyaluronidase. In some embodiments, the subcutaneous administration takes no more than 10 minutes. In some embodiments, the treatment achieves the result of being non-inferior to an intravenous infusion of said anti-CD20 antibody (e.g., at a dose of 600 mg), as measured by pharmacokinetics (levels in the blood, e.g., serum area under the curve (AUC)) over 12 weeks. In some embodiments, the treatment achieves the result of having comparable MRI lesion activity in the brain of the patient over 12 weeks to an intravenous infusion of said anti-CD20 antibody (e.g., at a dose of 600 mg). In some embodiments, the treatment achieves the result of a rapid, complete and sustained B-cell depletion. In some embodiments, the treatment achieves a safety profile consistent with that of an intravenous infusion of said anti-CD20 antibody (e.g., at a dose of 600 mg). In some embodiments, the anti-CD20 antibody is in a pharmaceutical formulation, wherein the pharmaceutical formulation comprises about 920 mg of the anti-CD20 antibody, about 23,000 units of hyaluronidase, about 2.088 g of α,α-trehalose dihydrate, about 5.5 mg of glacial acetic acid, about 34.4 mg of L-methionine, about 13.8 mg of polysorbate 20, about 50.1 mg of sodium acetate trihydrate, and water for injection at a pH of about 5.3. In some embodiments, the anti-CD20 antibody is ocrelizumab. In some embodiments, the anti-CD20 antibody is supplied in a 50 mL single-dose vial. In some embodiments, the formulation is in a volume of 23 mL.

In some embodiments, there is provided a method of treating multiple sclerosis in a patient comprising subcutaneously administering an anti-CD20 antibody to the patient at a dose of about 920 mg, wherein the anti-CD20 antibody comprises a V_{H} domain comprising the amino acid sequence set forth in SEQ ID NO: 8, a V_{L} domain comprising the amino acid sequence set forth in SEQ ID NO: 7, and a human IgG1 constant region, wherein the patient has been subjected to a prior administration (e.g., a prior intravenous administration) of the anti-CD20 antibody (e.g., at a dose of about 600 mg). In some embodiments, the anti-CD20 antibody comprises a light chain comprising the amino acid sequence of SEQ ID NO: 9 and a heavy chain comprising the amino acid sequence of SEQ ID NO: 10 or 11. In some embodiments, the anti-CD20 antibody is ocrelizumab. In some embodiments, the multiple sclerosis is relapsing multiple sclerosis (RMS). In some embodiments, the patient has a clinical isolated syndrome (CIS), relapsing-remitting multiple sclerosis (RRMS) or active secondary progressive multiple sclerosis (SPMS). In some embodiments, the multiple sclerosis is a primary progressive multiple sclerosis (PPMS). In some embodiments, the patient has been subject to oral dexamethasone (or an equivalent corticosteroid) and antihistamine shortly prior to the subcutaneous administration of the anti-CD20 antibody, optionally the oral dexamethasone (or the equivalent corticosteroid) and antihistamine are administered within 30 minutes, 20 minutes, or 15 minutes prior to the subcutaneous administration. In some embodiments, the anti-CD20 antibody is the only medicament administered to the patient to treat multiple sclerosis. In some embodiments, the anti-CD20 antibody is comprised in a liquid formulation at the concentration of about 40 mg/ml. said formulation further comprises a buffering agent, a stabilizer and surfactant, optionally wherein the formulation further comprises a hyaluronidase. In some embodiments, the buffering agent comprises sodium acetate, optionally wherein the formulation comprises about 20 mM sodium acetate providing a pH of about 5.3. In some embodiments, the stabilizer comprises trehalose and/or methionine, optionally wherein the formulation comprises a) about 240 mM trehalose and b) about 10 mM methionine. In some embodiments, the surfactant comprises polysorbate 20, and optionally wherein the formulation comprises about 0.06% (w/v) polysorbate 20. In some embodiments, the hyaluronidase comprises a recombinant human hyaluronidase, optionally the recombinant human hyaluronidase is rHuPH20, further optionally the formulation comprises about 1000 U/ml hyaluronidase. In some embodiments, the formulation comprises about 23,000 units of hyaluronidase. In some embodiments, the anti-CD20 antibody is comprised in a liquid formulation at the concentration of about 35-45 mg/ml (e.g., 40 mg/ml). In some embodiments, said formulation further comprises a buffering agent, a stabilizer and surfactant, optionally wherein the formulation further comprises a hyaluronidase. In some embodiments, the buffering agent comprises sodium acetate, optionally wherein the formulation comprises about 20 mM sodium acetate providing a pH of about 5 to about 5.6 (e.g., 5.3). In some embodiments, the stabilizer comprises trehalose and/or methionine, optionally wherein the formulation comprises a) about 190 mM to about 290 mM (e.g., 240 mM) trehalose and b) about 5 mM to about 15 mM (e.g., 10 mM) methionine. In some embodiments, the surfactant comprises polysorbate 20, and optionally wherein the formulation comprises about 0.04-0.08% (w/v) (e.g., 0.06% (w/v)) polysorbate 20. In some embodiments, the hyaluronidase comprises a recombinant human hyaluronidase, optionally the recombinant human hyaluronidase is rHuPH20, further optionally the formulation comprises about 1000 U/ml hyaluronidase. In some embodiments, the formulation comprises about 23,000 units of hyaluronidase. In some embodiments, the subcutaneous administration takes no more than 10 minutes. In some embodiments, the treatment achieves the result of being non-inferior to an intravenous infusion of said anti-CD20 antibody (e.g., at a dose of 600 mg), as measured by pharmacokinetics (levels in the blood, e.g., serum area under the curve (AUC)) over 12 weeks. In some embodiments, the treatment achieves the result of having comparable MRI lesion activity in the brain of the patient over 12 weeks to an intravenous infusion of said anti-CD20 antibody (e.g., at a dose of 600 mg). In some embodiments, the treatment achieves the result of a rapid, complete and sustained B-cell depletion. In some embodiments, the treatment achieves a safety profile consistent with that of an intravenous infusion of said anti-CD20 antibody (e.g., at a dose of 600 mg). In some embodiments, the anti-CD20 antibody is in a pharmaceutical formulation, wherein the pharmaceutical formulation comprises about 920 mg of the anti-CD20 antibody, about 23,000 units of hyaluronidase, about 2.088 g of α,α-trehalose dihydrate, about 5.5 mg of glacial acetic acid, about 34.4 mg of L-methionine, about 13.8 mg of polysorbate 20, about 50.1 mg of sodium acetate trihydrate, and water for injection at a pH of about 5.3. In some embodiments, the anti-CD20 antibody is ocrelizumab. In some embodiments, the anti-CD20 antibody is supplied in a 50 mL single-dose vial. In some embodiments, the formulation is in a volume of 23 mL.

In some embodiments, there is provided a method of treating multiple sclerosis in a patient comprising subcutaneously administering an anti-CD20 antibody to the patient a dose or at least two doses (such as two, three, or four) of about 920 mg, wherein the anti-CD20 antibody comprises a V_{H} domain comprising the amino acid sequence set forth in SEQ ID NO: 8, a V_{L} domain comprising the amino acid sequence set forth in SEQ ID NO: 7, and a human IgG1 constant region, wherein the patient has not been subjected to a prior subcutaneous administration of the anti-CD20 antibody at a dose lower than about 900 mg. In some embodiments, the anti-CD20 antibody comprises a light chain comprising the amino acid sequence of SEQ ID NO: 9 and a heavy chain comprising the amino acid sequence of SEQ ID NO: 10 or 11. In some embodiments, the anti-CD20 antibody is ocrelizumab. In some embodiments, the multiple sclerosis is relapsing multiple sclerosis (RMS). In some embodiments, the patient has a clinical isolated syndrome (CIS), relapsing-remitting multiple sclerosis (RRMS) or active secondary progressive multiple sclerosis (SPMS). In some embodiments, the multiple sclerosis is a primary progressive multiple sclerosis (PPMS). In some embodiments, the patient has been subject to oral dexamethasone (or an equivalent corticosteroid) and antihistamine shortly prior to the subcutaneous administration of the anti-CD20 antibody, optionally the oral dexamethasone (or the equivalent corticosteroid) and antihistamine are administered within 30 minutes, 20 minutes, or 15 minutes prior to the subcutaneous administration. In some embodiments, the anti-CD20 antibody is the only medicament administered to the patient to treat multiple sclerosis. In some embodiments, the anti-CD20 antibody is comprised in a liquid formulation at the concentration of about 40 mg/ml. said formulation further comprises a buffering agent, a stabilizer and surfactant, optionally wherein the formulation further comprises a hyaluronidase. In some embodiments, the buffering agent comprises sodium acetate, optionally wherein the formulation comprises about 20 mM sodium acetate providing a pH of about 5.3. In some embodiments, the stabilizer comprises trehalose and/or methionine, optionally wherein the formulation comprises a) about 240 mM trehalose and b) about 10 mM methionine. In some embodiments, the surfactant comprises polysorbate 20, and optionally wherein the formulation comprises about 0.06% (w/v) polysorbate 20. In some embodiments, the hyaluronidase comprises a recombinant human hyaluronidase, optionally the recombinant human hyaluronidase is rHuPH20, further optionally the formulation comprises about 1000 U/ml hyaluronidase. In some embodiments, the formulation comprises about 23,000 units of hyaluronidase. In some embodiments, the anti-CD20 antibody is comprised in a liquid formulation at the concentration of about 35-45 mg/ml (e.g., 40 mg/ml). In some embodiments, said formulation further comprises a buffering agent, a stabilizer and surfactant, optionally wherein the formulation further comprises a hyaluronidase. In some embodiments, the buffering agent comprises sodium acetate, optionally wherein the formulation comprises about 20 mM sodium acetate providing a pH of about 5 to about 5.6 (e.g., 5.3). In some embodiments, the stabilizer comprises trehalose and/or methionine, optionally wherein the formulation comprises a) about 190 mM to about 290 mM (e.g., 240 mM) trehalose and b) about 5 mM to about 15 mM (e.g., 10 mM) methionine. In some embodiments, the surfactant comprises polysorbate 20, and optionally wherein the formulation comprises about 0.04-0.08% (w/v) (e.g., 0.06% (w/v)) polysorbate 20. In some embodiments, the hyaluronidase comprises a recombinant human hyaluronidase, optionally the recombinant human hyaluronidase is rHuPH20, further optionally the formulation comprises about 1000 U/ml hyaluronidase. In some embodiments, the formulation comprises about 23,000 units of hyaluronidase. In some embodiments, the subcutaneous administration takes no more than 10 minutes. In some embodiments, the treatment achieves the result of being non-inferior to an intravenous infusion of said anti-CD20 antibody (e.g., at a dose of 600 mg), as measured by pharmacokinetics (levels in the blood, e.g., serum area under the curve (AUC)) over 12 weeks. In some embodiments, the treatment achieves the result of having comparable MRI lesion activity in the brain of the patient over 12 weeks to an intravenous infusion of said anti-CD20 antibody (e.g., at a dose of 600 mg). In some embodiments, the treatment achieves the result of a rapid, complete and sustained B-cell depletion. In some embodiments, the treatment achieves a safety profile consistent with that of an intravenous infusion of said anti-CD20 antibody (e.g., at a dose of 600 mg). In some embodiments, the anti-CD20 antibody is in a pharmaceutical formulation, wherein the pharmaceutical formulation comprises about 920 mg of the anti-CD20 antibody, about 23,000 units of hyaluronidase, about 2.088 g of α,α-trehalose dihydrate, about 5.5 mg of glacial acetic acid, about 34.4 mg of L-methionine, about 13.8 mg of polysorbate 20, about 50.1 mg of sodium acetate trihydrate, and water for injection at a pH of about 5.3. In some embodiments, the anti-CD20 antibody is ocrelizumab. In some embodiments, the anti-CD20 antibody is supplied in a 50 mL single-dose vial. In some embodiments, the formulation is in a volume of 23 mL.

In some embodiments, there is provided a method of treating multiple sclerosis in a patient comprising subcutaneously administering an anti-CD20 antibody to the patient at least two, three or four doses of about 920 mg, wherein the anti-CD20 antibody comprises a V_{H} domain comprising the amino acid sequence set forth in SEQ ID NO: 8, a V_{L} domain comprising the amino acid sequence set forth in SEQ ID NO: 7, and a human IgG1 constant region, wherein the anti-CD20 antibody is administered at a frequency of no more than once every 24 weeks or about once every 24 weeks, or at a frequency of no more than once every 6 months or about once every 6 months. In some embodiments, the anti-CD20 antibody comprises a light chain comprising the amino acid sequence of SEQ ID NO: 9 and a heavy chain comprising the amino acid sequence of SEQ ID NO: 10 or 11. In some embodiments, the anti-CD20 antibody is ocrelizumab. In some embodiments, the multiple sclerosis is relapsing multiple sclerosis (RMS). In some embodiments, the patient has a clinical isolated syndrome (CIS), relapsing-remitting multiple sclerosis (RRMS) or active secondary progressive multiple sclerosis (SPMS). In some embodiments, the multiple sclerosis is a primary progressive multiple sclerosis (PPMS). In some embodiments, the patient has been subject to oral dexamethasone (or an equivalent corticosteroid) and antihistamine shortly prior to the subcutaneous administration of the anti-CD20 antibody, optionally the oral dexamethasone (or the equivalent corticosteroid) and antihistamine are administered within 30 minutes, 20 minutes, or 15 minutes prior to the subcutaneous administration. In some embodiments, the anti-CD20 antibody is the only medicament administered to the patient to treat multiple sclerosis. In some embodiments, the anti-CD20 antibody is comprised in a liquid formulation at the concentration of about 40 mg/ml. said formulation further comprises a buffering agent, a stabilizer and surfactant, optionally wherein the formulation further comprises a hyaluronidase. In some embodiments, the buffering agent comprises sodium acetate, optionally wherein the formulation comprises about 20 mM sodium acetate providing a pH of about 5.3. In some embodiments, the stabilizer comprises trehalose and/or methionine, optionally wherein the formulation comprises a) about 240 mM trehalose and b) about 10 mM methionine. In some embodiments, the surfactant comprises polysorbate 20, and optionally wherein the formulation comprises about 0.06% (w/v) polysorbate 20. In some embodiments, the hyaluronidase comprises a recombinant human hyaluronidase, optionally the recombinant human hyaluronidase is rHuPH20, further optionally the formulation comprises about 1000 U/ml hyaluronidase. In some embodiments, the formulation comprises about 23,000 units of hyaluronidase. In some embodiments, the anti-CD20 antibody is comprised in a liquid formulation at the concentration of about 35-45 mg/ml (e.g., 40 mg/ml). In some embodiments, said formulation further comprises a buffering agent, a stabilizer and surfactant, optionally wherein the formulation further comprises a hyaluronidase. In some embodiments, the buffering agent comprises sodium acetate, optionally wherein the formulation comprises about 20 mM sodium acetate providing a pH of about 5 to about 5.6 (e.g., 5.3). In some embodiments, the stabilizer comprises trehalose and/or methionine, optionally wherein the formulation comprises a) about 190 mM to about 290 mM (e.g., 240 mM) trehalose and b) about 5 mM to about 15 mM (e.g., 10 mM) methionine. In some embodiments, the surfactant comprises polysorbate 20, and optionally wherein the formulation comprises about 0.04-0.08% (w/v) (e.g., 0.06% (w/v)) polysorbate 20. In some embodiments, the hyaluronidase comprises a recombinant human hyaluronidase, optionally the recombinant human hyaluronidase is rHuPH20, further optionally the formulation comprises about 1000 U/ml hyaluronidase. In some embodiments, the formulation comprises about 23,000 units of hyaluronidase. In some embodiments, the subcutaneous administration takes no more than 10 minutes. In some embodiments, the treatment achieves the result of being non-inferior to an intravenous infusion of said anti-CD20 antibody (e.g., at a dose of 600 mg), as measured by pharmacokinetics (levels in the blood, e.g., serum area under the curve (AUC)) over 12 weeks. In some embodiments, the treatment achieves the result of having comparable MRI lesion activity in the brain of the patient over 12 weeks to an intravenous infusion of said anti-CD20 antibody (e.g., at a dose of 600 mg). In some embodiments, the treatment achieves the result of a rapid, complete and sustained B-cell depletion. In some embodiments, the treatment achieves a safety profile consistent with that of an intravenous infusion of said anti-CD20 antibody (e.g., at a dose of 600 mg). In some embodiments, the anti-CD20 antibody is in a pharmaceutical formulation, wherein the pharmaceutical formulation comprises about 920 mg of the anti-CD20 antibody, about 23,000 units of hyaluronidase, about 2.088 g of α,α-trehalose dihydrate, about 5.5 mg of glacial acetic acid, about 34.4 mg of L-methionine, about 13.8 mg of polysorbate 20, about 50.1 mg of sodium acetate trihydrate, and water for injection at a pH of about 5.3. In some embodiments, the anti-CD20 antibody is ocrelizumab. In some embodiments, the anti-CD20 antibody is supplied in a 50 mL single-dose vial. In some embodiments, the formulation is in a volume of 23 mL.

In some embodiments, there is provided a method of treating multiple sclerosis in a patient comprising subcutaneously administering an anti-CD20 antibody to the patient at least two, three or four doses of about 920 mg, wherein the anti-CD20 antibody comprises a V_{H} domain comprising the amino acid sequence set forth in SEQ ID NO: 8, a V_{L} domain comprising the amino acid sequence set forth in SEQ ID NO: 7, and a human IgG1 constant region, the anti-CD20 antibody is in a pharmaceutical formulation, wherein the pharmaceutical formulation comprises: a. about 40 mg/ml anti-CD20 antibody; b. about 20 mM sodium acetate providing a pH of about 5.3; c. about 240 mM trehalose; d. about 10 mM methionine; e. about 0.06% (w/v) polysorbate 20; and f. about 1000 U/ml hyaluronidase. In some embodiments, the anti-CD20 antibody comprises a light chain comprising the amino acid sequence of SEQ ID NO: 9 and a heavy chain comprising the amino acid sequence of SEQ ID NO: 10 or 11. In some embodiments, the anti-CD20 antibody is ocrelizumab. In some embodiments, the multiple sclerosis is relapsing multiple sclerosis (RMS). In some embodiments, the patient has a clinical isolated syndrome (CIS), relapsing-remitting multiple sclerosis (RRMS) or active secondary progressive multiple sclerosis (SPMS). In some embodiments, the multiple sclerosis is a primary progressive multiple sclerosis (PPMS). In some embodiments, the patient has been subject to oral dexamethasone (or an equivalent corticosteroid) and antihistamine shortly prior to the subcutaneous administration of the anti-CD20 antibody, optionally the oral dexamethasone (or the equivalent corticosteroid) and antihistamine are administered within 30 minutes, 20 minutes, or 15 minutes prior to the subcutaneous administration. In some embodiments, the anti-CD20 antibody is the only medicament administered to the patient to treat multiple sclerosis. In some embodiments, the subcutaneous administration takes no more than 10 minutes. In some embodiments, the treatment achieves the result of being non-inferior to an intravenous infusion of said anti-CD20 antibody (e.g., at a dose of 600 mg), as measured by pharmacokinetics (levels in the blood, e.g., serum area under the curve (AUC)) over 12 weeks. In some embodiments, the treatment achieves the result of having comparable MRI lesion activity in the brain of the patient over 12 weeks to an intravenous infusion of said anti-CD20 antibody (e.g., at a dose of 600 mg). In some embodiments, the treatment achieves the result of a rapid, complete and sustained B-cell depletion. In some embodiments, the treatment achieves a safety profile consistent with that of an intravenous infusion of said anti-CD20 antibody (e.g., at a dose of 600 mg). In some embodiments, the anti-CD20 antibody is in a pharmaceutical formulation, wherein the pharmaceutical formulation comprises about 920 mg of the anti-CD20 antibody, about 23,000 units of hyaluronidase, about 2.088 g of α,α-trehalose dihydrate, about 5.5 mg of glacial acetic acid, about 34.4 mg of L-methionine, about 13.8 mg of polysorbate 20, about 50.1 mg of sodium acetate trihydrate, and water for injection at a pH of about 5.3. In some embodiments, the anti-CD20 antibody is ocrelizumab. In some embodiments, the anti-CD20 antibody is supplied in a 50 mL single-dose vial. In some embodiments, the formulation is in a volume of 23 mL.

In some embodiments, there is provided a method of treating multiple sclerosis in a patient comprising subcutaneously administering an anti-CD20 antibody to the patient at least two, three or four doses of about 920 mg, wherein the anti-CD20 antibody comprises a V_{H} domain comprising the amino acid sequence set forth in SEQ ID NO: 8, a V_{L} domain comprising the amino acid sequence set forth in SEQ ID NO: 7, and a human IgG1 constant region, wherein the anti-CD20 antibody is administered at a frequency of no more than once every 24 weeks or about once every 24 weeks, or at a frequency of no more than once every 6 months or about once every 6 months, wherein the pharmaceutical formulation comprises: a. about 40 mg/ml anti-CD20 antibody; b. about 20 mM sodium acetate providing a pH of about 5.3; c. about 240 mM trehalose; d. about 10 mM methionine; e. about 0.06% (w/v) polysorbate 20; and f. about 1000 U/ml hyaluronidase. In some embodiments, the anti-CD20 antibody comprises a light chain comprising the amino acid sequence of SEQ ID NO: 9 and a heavy chain comprising the amino acid sequence of SEQ ID NO: 10 or 11. In some embodiments, the anti-CD20 antibody is ocrelizumab. In some embodiments, the multiple sclerosis is relapsing multiple sclerosis (RMS). In some embodiments, the patient has a clinical isolated syndrome (CIS), relapsing-remitting multiple sclerosis (RRMS) or active secondary progressive multiple sclerosis (SPMS). In some embodiments, the multiple sclerosis is a primary progressive multiple sclerosis (PPMS). In some embodiments, the patient has been subject to oral dexamethasone (or an equivalent corticosteroid) and antihistamine shortly prior to the subcutaneous administration of the anti-CD20 antibody, optionally the oral dexamethasone (or the equivalent corticosteroid) and antihistamine are administered within 30 minutes, 20 minutes, or 15 minutes prior to the subcutaneous administration. In some embodiments, the anti-CD20 antibody is the only medicament administered to the patient to treat multiple sclerosis. In some embodiments, the subcutaneous administration takes no more than 10 minutes. In some embodiments, the treatment achieves the result of being non-inferior to an intravenous infusion of said anti-CD20 antibody (e.g., at a dose of 600 mg), as measured by pharmacokinetics (levels in the blood, e.g., serum area under the curve (AUC)) over 12 weeks. In some embodiments, the treatment achieves the result of having comparable MRI lesion activity in the brain of the patient over 12 weeks to an intravenous infusion of said anti-CD20 antibody (e.g., at a dose of 600 mg). In some embodiments, the treatment achieves the result of a rapid, complete and sustained B-cell depletion. In some embodiments, the treatment achieves a safety profile consistent with that of an intravenous infusion of said anti-CD20 antibody (e.g., at a dose of 600 mg). In some embodiments, the anti-CD20 antibody is in a pharmaceutical formulation, wherein the pharmaceutical formulation comprises about 920 mg of the anti-CD20 antibody, about 23,000 units of hyaluronidase, about 2.088 g of α,α-trehalose dihydrate, about 5.5 mg of glacial acetic acid, about 34.4 mg of L-methionine, about 13.8 mg of polysorbate 20, about 50.1 mg of sodium acetate trihydrate, and water for injection at a pH of about 5.3. In some embodiments, the anti-CD20 antibody is ocrelizumab. In some embodiments, the anti-CD20 antibody is supplied in a 50 mL single-dose vial. In some embodiments, the formulation is in a volume of 23 mL.

In some embodiments, there is provided a method of treating multiple sclerosis in a patient comprising subcutaneously administering an anti-CD20 antibody to the patient at least two, three or four doses of about 920 mg, wherein the anti-CD20 antibody comprises a V_{H} domain comprising the amino acid sequence set forth in SEQ ID NO: 8, a V_{L} domain comprising the amino acid sequence set forth in SEQ ID NO: 7, and a human IgG1 constant region, the anti-CD20 antibody is in a pharmaceutical formulation, wherein the pharmaceutical formulation comprises: a. about 40 mg/ml anti-CD20 antibody; b. about 20 mM sodium acetate providing a pH of about 5.3; c. about 240 mM trehalose; d. about 10 mM methionine; e. about 0.06% (w/v) polysorbate 20; and f. about 1000 U/ml hyaluronidase, wherein the anti-CD20 antibody is administered at a frequency of no more than once every 24 weeks or about once every 24 weeks, or at a frequency of no more than once every 6 months or about once every 6 months. In some embodiments, the anti-CD20 antibody comprises a light chain comprising the amino acid sequence of SEQ ID NO: 9 and a heavy chain comprising the amino acid sequence of SEQ ID NO: 10 or 11. In some embodiments, the anti-CD20 antibody is ocrelizumab. In some embodiments, the multiple sclerosis is relapsing multiple sclerosis (RMS). In some embodiments, the patient has a clinical isolated syndrome (CIS), relapsing-remitting multiple sclerosis (RRMS) or active secondary progressive multiple sclerosis (SPMS). In some embodiments, the multiple sclerosis is a primary progressive multiple sclerosis (PPMS). In some embodiments, the patient has been subject to oral dexamethasone (or an equivalent corticosteroid) and antihistamine shortly prior to the subcutaneous administration of the anti-CD20 antibody, optionally the oral dexamethasone (or the equivalent corticosteroid) and antihistamine are administered within 30 minutes, 20 minutes, or 15 minutes prior to the subcutaneous administration. In some embodiments, the anti-CD20 antibody is the only medicament administered to the patient to treat multiple sclerosis. In some embodiments, the treatment achieves the result of being non-inferior to an intravenous infusion of said anti-CD20 antibody (e.g., at a dose of 600 mg), as measured by pharmacokinetics (levels in the blood, e.g., serum area under the curve (AUC)) over 12 weeks. In some embodiments, the treatment achieves the result of having comparable MRI lesion activity in the brain of the patient over 12 weeks to an intravenous infusion of said anti-CD20 antibody (e.g., at a dose of 600 mg). In some embodiments, the treatment achieves the result of a rapid, complete and sustained B-cell depletion. In some embodiments, the treatment achieves a safety profile consistent with that of an intravenous infusion of said anti-CD20 antibody (e.g., at a dose of 600 mg). In some embodiments, the anti-CD20 antibody is in a pharmaceutical formulation, wherein the pharmaceutical formulation comprises about 920 mg of the anti-CD20 antibody, about 23,000 units of hyaluronidase, about 2.088 g of a, α-trehalose dihydrate, about 5.5 mg of glacial acetic acid, about 34.4 mg of L-methionine, about 13.8 mg of polysorbate 20, about 50.1 mg of sodium acetate trihydrate, and water for injection at a pH of about 5.3. In some embodiments, the anti-CD20 antibody is ocrelizumab. In some embodiments, the anti-CD20 antibody is supplied in a 50 mL single-dose vial. In some embodiments, the formulation is in a volume of 23 mL.

In some embodiments, the treatment results in non-inferior efficacy as compared to that of an alternative treatment comprising an intravenous administration of an anti-CD20 antibody. In some embodiments, the dose of the anti-CD20 antibody for the alternative intravenous administration is about 600 mg. In some embodiments, the 600 mg of the anti-CD20 antibody is administered in two split administration of a dose of 300 mg each, and wherein the two split administrations are separated from each other by two weeks or at least two weeks.

In some embodiments, the non-inferior efficacy is determined by assessing an overall exposure of the anti-CD20 antibody. In some embodiments, the overall exposure of the anti-CD20 antibody is measured by a serum area under the curve (AUC) of the anti-CD20 antibody for the first 12 weeks after the administration. In some embodiments, the non-inferior efficacy is determined when the lower end of the two-sided 90% CI of the geometric mean ratio (GMR) of AUC is >0.8.

In some embodiments, the treatment results in a serum area under the curve (AUC) of the anti-CD20 antibody for the first 12 weeks after the administration of no less than about 1100, 1125, 1150 or 1175 day*mcg/mL. In some embodiments, the treatment results in a serum area under the curve (AUC) of the anti-CD20 antibody for the first 24 weeks after the administration of no less than about 1100, 1125, 1150, 1175, or 1200 day*mcg/mL.

In some embodiments, the treatment results in a serum area under the curve (AUC) of the anti-CD20 antibody for the first 12 weeks after the administration of no more than about 7000, 6750, 6500, 6250, or 6000 day*mcg/mL. In some embodiments, the treatment results in a serum area under the curve (AUC) of the anti-CD20 antibody for the first 12 weeks after the administration of no more than about 7500, 7250, 7000 or 6750 day*mcg/mL.

In some embodiments, the treatment results in a serum area under the curve (AUC) of the anti-CD20 antibody for the first 12 weeks after the administration of about 1200 day*mcg/mL to about 6000 day*mcg/mL.

In some embodiments, the treatment results in a serum area under the curve (AUC) of the anti-CD20 antibody for the first 24 weeks after the administration of about 1200 day*mcg/mL to about 6600 day*mcg/mL.

In some embodiments, the treatment results in a Cₘₐₓ of the anti-CD20 antibody of no less than about 20, 25, 30, or 35 mcg/mL. In some embodiments, the treatment results in a Cₘₐₓ of the anti-CD20 antibody of no more than about 230, 225, 220, 215, or 210 mcg/mL. In some embodiments, the treatment results in a Cₘₐₓ of the anti-CD20 antibody of about 35-210 mcg/mL.

In some embodiments, the treatment results in a sustained B-cell depletion (e.g., at least about 60%, 70%, 80%, 90%, or 95% B cells are depleted) from about 2 weeks after administration to about 4 weeks, 8 weeks, 12 weeks, 16 weeks, 20 weeks or 24 weeks after administration. In some embodiments, the treatment results in a complete B-cell depletion (at least 95%, 96%, 97%, 98% or 99% B cells are depleted) from about 2 weeks after administration to about 12 weeks or 15 weeks after administration. In some embodiments, the treatment results in at least 80% B-cell depletion from about 2 weeks after administration to about 24 weeks after administration. In some embodiments, the treatment results in at least 95%, 96%, 97%, 98% or 99% B-cell depletion at about 2 weeks, 4 weeks, 8 weeks, 12 weeks, or 16 weeks after administration. In some embodiments, the treatment results in at least 80% B-cell depletion at about 2 weeks, 4 weeks, 8 weeks, 12 weeks, 16 weeks, 20 weeks, or 24 weeks after administration. In some embodiments, the patient has been subjected to a prior B cell depletion therapy (e.g., an anti-CD20 antibody therapy). In some embodiments, the patient has not been subjected to a prior B cell depletion therapy (e.g., an anti-CD20 antibody therapy).

In some embodiments, the treatment results in a comparable B cell depletion effect (such as those described above) as compared to that of an alternative treatment comprising an intravenous administration of an anti-CD20 antibody. In some embodiments, the dose of the anti-CD20 antibody for the alternative intravenous administration is about 600 mg. In some embodiments, the 600 mg of the anti-CD20 antibody is administered in two split administration of a dose of 300 mg each, and wherein the two split administrations are separated from each other by two weeks or at least two weeks.

In some embodiments, the treatment results in complete suppression of MRI lesion (e.g., previous T1Gd+ lesions, e.g., new or enlarging T2 lesions) at week 8, 12, 16, 20, and 24 after administration. In some embodiments, the treatment results in an absence of relapse for at least 4, 8, 12, 16, 20, or 24 weeks after administration.

In some embodiments, the treatment results in a comparable radiological effect as compared to that of an alternative treatment comprising an intravenous administration of an anti-CD20 antibody. In some embodiments, the dose of the anti-CD20 antibody for the alternative intravenous administration is about 600 mg. In some embodiments, the 600 mg of the anti-CD20 antibody is administered in two split administration of a dose of 300 mg each, and wherein the two split administrations are separated from each other by two weeks or at least two weeks. In some embodiments, the radiological effect comprises the total number of T1Gd+ lesions as detected by brain MRI at weeks 8 and/or 24 relative to previous scan. In some embodiments, the radiological effect comprises the results the total number of new or enlarging T2 lesions as detected by brain MRI at weeks 12 and/or 24.

In some embodiments, the treatment results in a comparable safety profile as compared to that of an alternative treatment comprising an intravenous administration of an anti-CD20 antibody. In some embodiments, the dose of the anti-CD20 antibody for the alternative intravenous administration is about 600 mg. In some embodiments, the 600 mg of the anti-CD20 antibody is administered in two split administration of a dose of 300 mg each, and wherein the two split administrations are separated from each other by two weeks or at least two weeks. In some embodiments, the comparable safety profile is determined by the similar lack of anti-drug antibodies in the individual after treatment (e.g., antibodies against the anti-CD20 antibody, e.g., antibodies against rHuPH20). In some embodiments, the comparable safety profile is determined by a similar serious adverse event risk (e.g., based upon a prior clinical trial results, e.g., similar rates of SAE post treatment). In some embodiments, the comparable safety profile is determined by the lack of safety concern based upon a prior clinical trial results, e.g., similar rates of SAE post treatment.

A. Antibodies and their Production

The methods and articles of manufacture of the present application use, or incorporate, an antibody that binds to a B-cell surface marker, especially one that binds to CD20. Accordingly, methods for generating such antibodies are described here.

In some embodiments, the anti-CD20 antibody used in the methods described here is produced by a method comprising expressing a nucleic acid encoding a humanized antibody comprising the heavy and light chain amino acid sequences of SEQ ID NO:10 or 11, and SEQ ID NO:9, respectively, in a host cell, and recovering the humanized antibody or an antigen-binding fragment thereof expressed in the host cell. In some embodiments, the host cell is a mammalian cell (e.g., a CHO cell), an insect cell, or a plant cell. In some embodiments the host cell is a bacterial cell. Methods of producing an anti-CD20 are described in further detail in, e.g., U.S. Pat. No. 7,799,900.

The B cell surface marker to be used for production of, or screening for, antibodies may be, e.g., a soluble form of the marker or a portion thereof, containing the desired epitope. Alternatively, or additionally, cells expressing the marker at their cell surface can be used to generate, or screen for, antibodies. Other forms of the B cell surface marker useful for generating antibodies are apparent to those skilled in the art.

A description follows as to exemplary techniques for the production of the antibodies used in accordance with the present application.

### Humanized Antibodies

Methods for humanizing non-human antibodies have been described in the art. In some embodiments, a humanized antibody has one or more amino acid residues introduced into it from a source that is non-human. These non-human amino acid residues are often referred to as “import” residues, which are typically taken from an “import” variable domain. Humanization can be essentially performed following the method of Winter and co-workers (Jones et al., Nature, 321:522-525 (1986); Riechmann et al., Nature, 332:323-327 (1988); Verhoeyen et al., Science, 239:1534-1536 (1988)), by substituting hypervariable region sequences for the corresponding sequences of a human antibody. Accordingly, such “humanized” antibodies are chimeric antibodies (U.S. Pat. No. 4,816,567) wherein substantially less than an intact human variable domain has been substituted by the corresponding sequence from a non-human species. In practice, humanized antibodies are typically human antibodies in which some hypervariable region residues and possibly some FR residues are substituted by residues from analogous sites in rodent antibodies.

The choice of human variable domains, both light and heavy, to be used in making the humanized antibodies is very important to reduce antigenicity. According to the so-called “best-fit” method, the sequence of the variable domain of a rodent antibody is screened against the entire library of known human variable-domain sequences. The human sequence that is closest to that of the rodent is then accepted as the human framework region (FR) for the humanized antibody (Sims et al., J. Immunol., 151:2296 (1993); Chothia et al., J. Mol. Biol., 196:901 (1987)). Another method uses a particular framework region derived from the consensus sequence of all human antibodies of a particular subgroup of light or heavy chain variable regions. The same framework may be used for several different humanized antibodies (Carter et al., Proc. Natl. Acad. Sci. USA, 89:4285 (1992); Presta et al., J. Immunol., 151:2623 (1993)).

It is further important that antibodies be humanized with retention of high affinity for the antigen and other favorable biological properties. To achieve this goal, in some embodiments of the methods, humanized antibodies are prepared by a process of analysis of the parental sequences and various conceptual humanized products using three-dimensional models of the parental and humanized sequences. Three-dimensional immunoglobulin models are commonly available and are familiar to those skilled in the art. Computer programs are available that illustrate and display probable three-dimensional conformational structures of selected candidate immunoglobulin sequences. Inspection of these displays permits analysis of the likely role of the residues in the functioning of the candidate immunoglobulin sequence, i.e., the analysis of residues that influence the ability of the candidate immunoglobulin to bind its antigen. In this way, FR residues can be selected and combined from the recipient and import sequences so that the desired antibody characteristic, such as increased affinity for the target antigen(s), is achieved. In general, the hypervariable region residues are directly and most substantially involved in influencing antigen binding.

In some embodiments, the anti-CD20 antibody comprises a humanized anti-CD20 antibody. In some embodiments, the humanized anti-CD20 antibody comprises a light chain variable region comprising CDR L1 having the amino acid sequence set forth in SEQ ID NO: 1, CDR L2 having the amino acid sequence set forth in SEQ ID NO: 2, and CDR L3 having the amino acid sequence set forth in SEQ ID NO: 3, and a heavy chain variable region comprising CDR H1 having the amino acid sequence set forth in SEQ ID NO: 4, CDR H2 having the amino acid sequence set forth in SEQ ID NO: 5, and CDR H3 having the amino acid sequence set forth in SEQ ID NO: 6, and optionally a human IgG1 constant region.

In some embodiments, the anti-CD20 antibody comprises a V_{L} domain comprising the amino acid sequence set forth in SEQ ID NO: 7, and a V_{H} domain comprising the amino acid sequence set forth in SEQ ID NO: 8, and a human IgG1 constant region.

In some embodiments, the anti-CD20 antibody comprises a light chain comprising the amino acid sequence set forth in SEQ ID NO: 9, and a heavy chain comprising the amino acid sequence set forth in SEQ ID NO: 10, and a human IgG1 constant region.

In some embodiments, the anti-CD20 antibody comprises a light chain comprising the amino acid sequence set forth in SEQ ID NO: 9, and a heavy chain comprising the amino acid sequence set forth in SEQ ID NO: 11, and a human IgG1 constant region.

In some embodiments, the anti-CD20 antibody is ocrelizumab.

In some embodiments, the amino acid lysine at the C-terminus of the heavy chain of the anti-CD20 antibody is absent.

### B. Patient

The present invention in some aspects provides a method of treating MS in a patient.

In some embodiments, the patient has an EDSS score of 0-6.5. In some embodiments, the patient has a disease duration from onset of MS symptoms of no more than 15 years. In some embodiments, the patient has an EDSS score of less than 2. The EDSS is a commonly used measure for quantifying changes in the disability level of patients with MS over time. The EDSS is a disability scale that ranges in 0.5-point steps from 0 (normal) to 10.0 (death) (see Kurtzke (1983) Neurol 1983; 33:1444-52; and Kappos (2011) Neurology, University Hospital Basel, Switzerland: Neurostatus Scoring Definitions). The EDSS is based on a standard neurological examination, incorporating functional systems (visual, brainstem, pyramidal, cerebellar, sensory, bowel and bladder, and cerebral [or mental]) that are rated and then scored as a FSS (functional system score), and ambulation, which is scored as ambulation score. Each FSS is an ordinal clinical rating scale ranging from 0 to 5 or 6 and an ambulation score that is rated from 0 to 16. These ratings are then used in conjunction with observations, as well as information, concerning ambulation and use of assistive devices to determine the total EDSS score. In some embodiments, the EDSS is administered according to the criteria and calculated according to the algorithm described in D'Souza M, Yaldizli Ö, John R, et al. Neurostatus e-Scoring improves consistency of Expanded Disability Status Scale assessments: A proof of concept study. Mult Scler Houndmills Basingstoke Engl. 2017; (4): 597-603.

In some embodiments, the individual has Primary Progressive Multiple Sclerosis (PPMS). In some embodiments, the individual has a relapsing form of MS (RMS). In some embodiments, the MS is either an active or non-active form of MS based on the presence or absence of disease activity.

In some embodiments, the patient does not have an active hepatitis B infection. In some embodiments, hepatitis B virus (HBV) screening is performed on the patient before initiation of treatment with the anti-CD20 antibody described herein. In some embodiments, the patient is negative for surface antigen [HBsAg] and negative for HB core antibody [HBcAb+]. In some embodiments, the patient is negative for surface antigen [HBsAg] and positive for HB core antibody [HBcAb+] or is a carrier of HBV [HBsAg+]. In some embodiments, the patient is selected based upon a) not having an active hepatitis B infection, b) being negative for surface antigen [HBsAg] and negative for HB core antibody [HBcAb+], and/or c) being negative for surface antigen [HBsAg] and positive for HB core antibody [HBcAb+] or is a carrier of HBV [HBsAg+].

### Relapsing Multiple Sclerosis (RMS)

In some embodiments, the multiple sclerosis is relapsing multiple sclerosis (RMS). In some embodiments, the patient has been diagnosed with RMS according to the criteria described in Thompson et al. (2018) Lancet Neurol. 17:162-73. In some embodiments, the patient has RMS, and the treatment described herein achieves one or more of the results described herein (e.g., non-inferiority as measured by pharmacokinetics, controlling MRI lesion activity in the brain, B-cell depletion, consistent safety profile).

In some embodiments, the patient has been diagnosed with RMS in accordance with the revised McDonald Criteria 2017 (Thompson A J, Banwell B L, Barkhof F, et al. Diagnosis of multiple sclerosis: 2017 revisions of the McDonald criteria. Lancet Neurol 2018; 17:162-73). In some embodiments, the patient with RMS has not received prior treatment with an anti-CD20 antibody or has not received aprior anti-CD20 antibody in the past two years. In some embodiments, the patient with RMS has received prior treatment with an anti-CD20 antibody, and the last dose of anti-CD20 antibody was more than about two years prior to the start of treatment according to a method herein. In some embodiments, the patient with RMS has received prior treatment with an anti-CD20 antibody, and the patient has normal B-cell count. In some embodiments, the patient with RMS has received prior treatment with an anti-CD20 antibody, and the treatment was not discontinued due to lack of efficacy and/or adverse event. In some embodiments, the patient with RMS received prior treatment with rituximab, ocrelizumab, obinutuzumab, veltuzumab, tositumomab, ibritumomab, and/or ofatumumab. In some embodiments, the patient with RMS has not received prior treatment with mitoxantrone, cladribine, atacicept, and/or alemtuzumab.

### Progressive Multiple Sclerosis (PPMS)

In some embodiments, the multiple sclerosis is primary progressive multiple sclerosis (PPMS). In some embodiments, the patient has been diagnosed PPMS according to the criteria described in Thompson et al. (2018) Lancet Neurol. 17:162-73. In some embodiments, the patient has PPMS, and the treatment described herein achieves one or more of the results described herein (e.g., non-inferiority as measured by pharmacokinetics, controlling MRI lesion activity in the brain, B-cell depletion, consistent safety profile). In some embodiments, the patient is at least 18 years old.

In some embodiments, the patient with PPMS has not received prior treatment with an anti-CD20 antibody or has not received a prior anti-CD20 antibody in the past two years. In some embodiments, the patient with PPMS has received prior treatment with an anti-CD20 antibody, and the last dose of anti-CD20 antibody was more than about two years prior to the start of treatment according to a method herein. In some embodiments, the patient with PPMS has received prior treatment with an anti-CD20 antibody, and the patient has normal B-cell count. In some embodiments, the patient with PPMS has received prior treatment with an anti-CD20 antibody, and the treatment was not discontinued due to lack of efficacy and/or adverse event. In some embodiments, the patient with PPMS has received prior treatment with ocrelizumab. In some embodiments, the patient with RMS received prior treatment with rituximab, ocrelizumab, binutuzumab, veltuzumab, tositumomab, ibritumomab, ofatumumab. In some embodiments, the patient with RMS has not received prior treatment with mitoxantrone, cladribine, atacicept, and/or alemtuzumab.

In some embodiments, the patient has been diagnosed with PPMS in accordance with the revised McDonald Criteria 2017 (Thompson A J, Banwell B L, Barkhof F, et al. Diagnosis of multiple sclerosis: 2017 revisions of the McDonald criteria. Lancet Neurol 2018; 17:162-73). In some embodiments, the patient has an EDSS score between 3 to 6.5, inclusive, at the start of treatment (e.g., prior to the first dose of anti-CD20 antibody). In some embodiments, the patient has a score of ≥2.0 on the Functional Systems (FS) scale for the pyramidal system that is due to lower extremity findings. In some embodiments, the patient has a disease duration of less than about 15 years from onset of MS symptoms with an EDSS score of >5.0 at the start of treatment (e.g., prior to the first dose of anti-CD20 antibody). In some embodiments, the patient has a disease duration of less than about 10 years from the onset of MS symptoms with an EDSS score at screening of ≤5.0. IN some embodiments, the patient has documented evidence of the presence of cerebrospinal fluid-specific oligoclonal bands.

### C. Dosing Regimen

The following section describes various aspects (embodiments) of dosing and treatment regimens, any and all of which apply to the methods described herein.

The present application in one aspect provides a method of treating multiple sclerosis in an individual comprising administering an effective amount of an anti-CD20 antibody subcutaneously every six months or 24 weeks. In some embodiments, about 850 mg to about 1000 mg, about 900 mg to about 950 mg, or about 920 mg of an anti-CD20 antibody is administered. In some embodiments, about 920 mg of an anti-CD20 antibody is administered.

In some embodiments, the anti-CD20 antibody is administered subcutaneously by a healthcare professional to an individual with a manual syringe or syringe pump. In some embodiments, the anti-CD20 antibody is administered through a SC infusion set into the abdominal SC space, except for the 5 cm area directly around the navel, about every six months or 24 weeks. In some embodiments, a minimum of about 22 weeks or about five months is maintained between each subcutaneous dose of anti-CD20 antibody. In some embodiments, the anti-CD20 antibody is administered at home. In some embodiments, the anti-CD20 antibody is administered in a healthcare facility.

Additional SC spaces may be used for SC administration. These spaces include the outer area of the upper arm, the front of the thigh, midway to the outer side, 4 inches below the top of the thigh to 4 inches above the knee, the upper back, and the lower back in the upper area of the buttock, just behind the hip bone, this has the slowest rate of absorption among sites.

In some embodiments, provided herein are methods of treating individuals with MS comprising administering an effective amount an antibody that binds to CD20 comprising a variable heavy chain (VH) and a variable light chain (VL), wherein the VH comprises a CDRL1 comprising the amino acid sequence set forth in SEQ ID NO: 1, a CDRL2 comprising the amino acid sequence set forth in SEQ ID NO: 2, a CDRL3 comprising the amino acid sequence set forth in SEQ ID NO: 3, a CDRH1 comprising the amino acid sequence set forth in SEQ ID NO: 4, a CDRH2 comprising the amino acid sequence set forth in SEQ ID NO: 5, a CDRH3 comprising the amino acid sequence set forth in SEQ ID NO: 6 to the individual. In some embodiments, the CD20 antibody has a V_{L} domain comprising the amino acid sequence set forth in SEQ ID NO: 7 and a V_{H} domain comprising the amino acid sequence set forth in SEQ ID NO: 8. In some embodiments, the antibody comprises a light chain comprising the amino acid sequence set forth in SEQ ID NO: 9 and a heavy chain comprising the amino acid sequence set forth in SEQ ID NO: 10. In some embodiments, the antibody is ocrelizumab (CAS Registry No. 637334-45-3). In some embodiments, the antibody has an IgG1 isotype. In some embodiments, the antibody is a humanized antibody. In some embodiments, the antibody is a full-length antibody. In some embodiments, the antibody that bids to CD20 inhibits CD20. In some embodiments, the antibody that binds to CD20 selectively targets and depletes CD-20 expressing B cells, while preserving the capacity of B-cell reconstitution and preexisting humoral immunity. In some embodiments, the antibody that binds to CD20 is administered at a dose of about 920 mg. In some embodiments, the antibody that binds to CD20 is administered about every 6 months or 24 weeks. In some embodiments, the antibody that binds to CD20 is administered at least twice, at least three times, at least four times or at least five times. In some embodiments, the antibody that binds to CD20 is administered about every six months or about every 24 weeks for about 24 months. In some embodiments the individual has secondary progressive multiple sclerosis (SPMS). In some embodiments the individual has relapsing remitting multiple sclerosis (RRMS). In some embodiments, the individual has relapsing MS (RMS).

In some embodiments, to reduce potential injection reactions, about 20 mg of dexamethasone (or an equivalent corticosteroid) and/or about 5 mg of desloratadine are administered to the individual prior to subcutaneous administration of an anti-CD20 antibody. In some embodiments, 20 mg of dexamethasone (or an equivalent corticosteroid) and 5 mg of desloratadine are administered to the individual about 30 min to about 60 minutes, about 30 minutes to about 40 minutes, about 40 minutes to about 50 minutes, or about 50 minutes to about 60 minutes prior to subcutaneous administration of an anti-CD20 antibody. In some embodiments an equivalent dose of an alternative steroid or antihistaminic is administered to the individual prior to subcutaneous administration of an anti-CD20 antibody.

In some embodiments, an anti-CD20 antibody is administered in a formulation suitable for subcutaneous administration. In some embodiments, the anti-CD20 antibody is formulated in any of the formulations described herein. In some embodiments, the anti-CD20 antibody is in a pharmaceutical formulation, wherein the pharmaceutical formulation comprises about 30-350 mg/ml (e.g., about 40 mg/ml) anti-CD20 antibody; about 1-100 mM of a buffering agent (e.g., 20 mM sodium acetate) providing a pH of about 3.5-7.5 (e.g., about 5.3); about 1-500 mM of a stabilizer or a mixture of two or more stabilizers (e.g., about 240 mM trehalose and/or about 10 mM methionine); about 0.01-0.1% (w/v) of a nonionic surfactant (e.g., 0.06% (w/v) polysorbate 20); and optionally an effective amount of at least one hyaluronidase (e.g., about 1000 U/ml of hyaluronidase). In some embodiments, the pharmaceutical formulation additionally comprises recombinant human hyaluronidase. In some embodiments, the recombinant human hyaluronidase comprises rHuPH20. In some embodiments, the anti-CD20 antibody is in a pharmaceutical formulation, wherein the pharmaceutical formulation comprises about 920 mg of the anti-CD20 antibody, about 23,000 units of hyaluronidase, about 2.088 g of α,α-trehalose dihydrate, about 5.5 mg of glacial acetic acid, about 34.4 mg of L-methionine, about 13.8 mg of polysorbate 20, about 50.1 mg of sodium acetate trihydrate, and water for injection at a pH of about 5.3. In some embodiments, the anti-CD20 antibody is ocrelizumab. In some embodiments, the anti-CD20 antibody is supplied in a 50 mL single-dose vial. In some embodiments, the formulation is in a volume of 23 mL.

In some embodiments, an anti-CD20 antibody is administered multiple times (e.g., at least two times, or at least three times) over a period of 12 months or more. In some embodiments, an anti-CD20 antibody is administered multiple times (e.g., at least two, three, four times) over a period of 18 months or more. In some embodiments, an anti-CD20 antibody is administered multiple times (e.g., at least two, three, four or five times) over a period of 24 months or more.

It may be advantageous to deliver an anti-CD20 antibody to the individual more than once. For example, in some embodiments, the anti-CD20 antibody is administered at least twice, at least 3 times, at least 4 times, or at least 5 times. For example, in some embodiments, an anti-CD20 antibody is administered twice, 3 times, 4 times, or 5 times. In some embodiments, an anti-CD20 antibody is administered multiple times over a period of 24 months, one month to 24 months, 6 months to 24 months, or 12 months to 24 months.

In some aspects, provided herein is a method of treating MS in an individual comprising administering an anti-CD20 antibody subcutaneously to the individual about every six months or 24 weeks for about at least 12 months, at least 18 months, or at least 24 months; wherein each administration of the anti-CD20 antibody comprises a subcutaneous administration of about 920 mg of an anti-CD20 antibody.

In some embodiments, the method comprises (i) administering an anti-CD20 antibody subcutaneously to the individual on day 0, (ii) administering a second dose of the anti-CD20 antibody subcutaneously about 6 months (e.g., 180, 181, 182, 183, 184, or 185 days) later, (iii) administering a third dose of the anti-CD20 antibody subcutaneously about 12 months later, (iv) administering a fourth dose of the anti-CD20 antibody subcutaneously about 18 months later, (v) administering a fifth dose of the anti-CD20 antibody subcutaneously about 24 months later. In some embodiments, about 920 mg of the anti-CD20 antibody is administered during each dose.

In some embodiments, the anti-CD20 antibody is the only medicament administered to the individual to treat multiple sclerosis. In some embodiments, the anti-CD20 antibody is the only disease modifying therapy (DMT) administered to the patient to treat multiple sclerosis. For example, in some embodiments, the anti-CD20 antibody is administered in combination with one or more of: methylprednisolone (or equivalent); an antihistamine (e.g., diphenhydramine or equivalent); an analgesic (e.g., acetaminophen); and an antipyretic.

In some embodiments, the anti-CD20 antibody is administered via a device suitable for subcutaneous administration of a volume of at least 20 mL. In some embodiments, the anti-CD20 antibody is administered via a device suitable for subcutaneous administration of a volume of about 23 mL. In some embodiments, the device comprises an infusion pump. In some embodiments, the device comprises a CRONO ambulatory infusion pump. In some embodiments, the device comprises a Lapas patch pump. In some embodiments, the device comprises an enFuse® on-body platform. In some embodiments, the device comprises a wearable injector or wearable injection device. In some embodiments, the device comprises a DrugDeliverySystem®. See e.g., Badkar et al., Drug Des Devel Ther. 2021 Jan. 13; 15:159-170.

In some embodiments, provided herein are methods of treating individuals with multiple sclerosis by administering an anti-CD20 antibody subcutaneously to the individual, wherein each subcutaneous anti-CD20 antibody administration comprises delivering about 920 mg of the anti-CD20 antibody SC. In some embodiments the individual has secondary progressive multiple sclerosis (SPMS). In some embodiments the individual has relapsing remitting multiple sclerosis (RRMS). In some embodiments, the individual has relapsing MS (RMS).

Also provided herein is a method of treating MS in an individual comprising a dosing schedule of at least 24 months comprising administering about 920 mg of an anti-CD20 antibody SC about every 6 months or 24 weeks. In some embodiments, consecutive doses of the anti-CD20 antibody is administered at least about 22 weeks or about five months apart.

In some embodiments, the method comprises administering an anti-CD20 antibody on weeks 0, 26, 52, 78, and 104. In some embodiments, the method comprises administering an anti-CD20 antibody on weeks 0, 24, 48, 72, and 96. In some embodiments, administering an anti-CD20 antibody comprises a subcutaneous injection by a healthcare professional to an individual with a manual syringe or syringe pump. In some embodiments, subcutaneous administration of an anti-CD20 antibody comprises about 920 mg the anti-CD20 antibody.

In some embodiments, provided herein is a method of treating MS in an individual comprising administering an anti-CD20 antibody subcutaneously, wherein the anti-CD20 antibody administration comprises delivering about 920 mg of the anti-CD20 antibody subcutaneously to the individual during an anti-CD20 antibody delivery period, wherein during the anti-CD20 antibody delivery period, there is an effective amount of anti-CD20 antibody in the serum of the individual for treating MS.

In some embodiments, the anti-CD20 antibody is subcutaneously administered in a composition comprising the anti-CD20 antibody at a volume of 23 mL (e.g., in a pre-filled syringe or a device comprising a needle). In some embodiments, the anti-CD20 antibody is subcutaneously administered in a composition comprising the anti-CD20 antibody at a volume of 23 mL, wherein the formulation comprises 920 mg ocrelizumab and 23,000 units hyaluronidase. In some embodiments, the anti-CD20 antibody is administered into the abdominal SC space, except for the 5 cm area directly around the navel. In some embodiments, the subcutaneous administration takes about 10 minutes. In some embodiments, the anti-CD20 antibody is administered about every 6 months or 24 weeks. In some embodiments, consecutive doses of the anti-CD20 antibody are administered at least about 22 weeks or about 5 months apart. In some embodiments, the anti-CD20 antibody is subcutaneously administered in a composition comprising the anti-CD20 antibody at a volume of 23 mL, wherein the formulation comprises 920 mg ocrelizumab and 23,000 units hyaluronidase, and the anti-CD20 antibody is administered into the abdominal SC space, except for the 5 cm area directly around the navel, wherein the administration takes about 10 minutes, and the anti-CD20 antibody is administered about every 6 months or 24 weeks.

### D. Premedication

In certain embodiments, the patient is premedicated prior to (e.g., shortly prior to) subcutaneous administration with an anti-CD20 antibody (e.g., any anti-CD20 antibody described herein). Administering one or more premedication into a patient “shortly” prior to the subcutaneous administration in some cases refers to administering the one or more premedication within about 2 hours, 1 hour, 45 minutes, 30 minutes, 20 minutes, 15 minutes, 10 minutes, or 5 minutes prior to the subcutaneous administration of the anti-CD20 antibody.

In certain embodiments, the patient is premedicated with methylprednisolone (or an equivalent, e.g., an alternative steroid) within approximately 30 minutes to an hour prior to each subcutaneous administration of anti-CD20 antibody. In certain embodiments, the patient is premedicated with methylprednisolone (or an equivalent) within about 30 minutes (e.g., within about 20 minutes or 15 minutes) prior to each subcutaneous administration of anti-CD20 antibody. In certain embodiments, the patient is premedicated with 100 mg IV methylprednisolone (or an equivalent) within approximately 30 minutes to an hour prior to each subcutaneous administration of anti-CD20 antibody. In certain embodiments, the patient is premedicated with 100 mg IV methylprednisolone (or an equivalent) within about 30 minutes (e.g., within about 20 minutes or 15 minutes) prior to each subcutaneous administration of anti-CD20 antibody.

In certain embodiments, the patient is additionally (or alternatively) premedicated with an antihistaminic drug (e.g. diphenhydramine) approximately 30-60 minutes before each subcutaneous administration of anti-CD20 antibody. In certain embodiments, the patient is additionally (or alternatively) premedicated with an antihistaminic drug (e.g. diphenhydramine) within about 30 minutes (e.g., within about 20 minutes or 15 minutes) before each subcutaneous administration of anti-CD20 antibody. In certain embodiments, the patient is additionally (or alternatively) premedicated with an antipyretic (e.g. acetaminophen/paracetamol) approximately 30-60 minutes before each subcutaneous administration of anti-CD20 antibody. In certain embodiments, the patient is additionally (or alternatively) premedicated with an antipyretic (e.g. acetaminophen/paracetamol) within about 30 minutes (e.g., within about 20 minutes or 15 minutes) before each subcutaneous administration of anti-CD20 antibody.

### E. Prior Treatment

In some embodiments, the patient has been subjected to a prior treatment for MS.

In some embodiments, the patient has not been subjected to a prior treatment for MS.

In some embodiments, the patient has not been subjected to a prior intravenous administration of an anti-CD20 antibody (such as any anti-CD20 antibody described herein) at a dose of about 600 mg. In some embodiments, the patient has not been subjected to a prior intravenous administration of an anti-CD20 antibody (such as any anti-CD20 antibody described herein).

In some embodiments, the patient has not been subjected to a prior subcutaneous administration of an anti-CD20 antibody such as any anti-CD20 antibody described herein) at a dose lower than about 900 mg.

In some embodiments, the patient has not been previously administered an anti-CD20 antibody (such as any anti-CD20 antibody described herein). In some embodiments, the patient has not been previously administered an anti-CD20 antibody (such as any anti-CD20 antibody described herein) within 2 years prior to the start of treatment with anti-CD20 antibody described herein. In some embodiments, the anti-CD20 antibody is ocrelizumab.

In some embodiments, the patient has received a prior treatment with an anti-CD20 antibody. In some embodiments, the prior treatment comprises an intravenous administration of the anti-CD20 antibody. In some embodiments, the prior treatment comprises the intravenous administration of the anti-CD20 antibody at a dose of about 600 mg. In some embodiments, the prior treatment is at least about 1 month, 2 months, 3 months, 6 months, 9 months, 12 months before the time when the methods described herein are conducted.

In some embodiments, the patient has not been subjected to a prior treatment with cladribine, atacicept, or alemtuzumab.

In some embodiments, the patient has not been subjected to a prior treatment with fingolimod, siponimod, ponesimod, or ozanimod within 6 weeks prior to the methods comprising subcutaneously administering the anti-CD20 antibody described herein.

In some embodiments, the patient has not been subjected to a prior treatment with interferons beta (1a or 1b), or glatiramer acetate within 2 weeks prior to the methods comprising subcutaneously administering the anti-CD20 antibody described herein.

In some embodiments, the patient has not been subjected to a prior treatment with natalizumab within 4.5 months prior to the methods comprising subcutaneously administering the anti-CD20 antibody described herein.

In some embodiments, the patient has not been subjected to a prior treatment with mitoxantrone within 2 years prior to the methods comprising subcutaneously administering the anti-CD20 antibody described herein.

### F. Combination Therapies

While the anti-CD20 antibody may be the only drug administered to the patient to treat the multiple sclerosis, one may optionally administer a second medicament, e.g., a second multiple sclerosis disease modifying agent (DMT), such as a cytotoxic agent, chemotherapeutic agent, immunosuppressive agent, cytokine, cytokine antagonist or antibody, growth factor, hormone, integrin, integrin antagonist or antibody (e.g. an LFA-1 antibody, or an alpha 4 integrin antibody such as natalizumab (TYSABRI®) available from Biogen Idec/Elan Pharmaceuticals, Inc) etc., with the antibody that binds a B cell surface marker (e.g. with the anti-CD20 antibody).

In some embodiments, the antibody is combined with an interferon class drug such as IFN-beta-1a (REBIF® and AVONEX®) or IFN-beta-1b (BETASERON®); an oligopeptide such a glatiramer acetate (COPAXONE®); a cytotoxic agent such as mitoxantrone (NOVANTRONE®), methotrexate, cyclophosphamide, chlorambucil, azathioprine; intravenous immunoglobulin (gamma globulin); lymphocyte-depleting therapy (e.g., mitoxantrone, cyclophosphamide, alemtuzumab (Campath®, LEMTRADA™), anti-CD4, cladribine, total body irradiation, bone marrow transplantation); corticosteroid (e.g. methylprednisolone, prednisone, dexamethasone, or glucorticoid), including systemic corticosteroid therapy; non-lymphocyte-depleting immunosuppressive therapy (e.g., mycophenolate mofetil (MMF) or cyclosporine); cholesterol-lowering drug of the “statin” class, which includes cerivastatin (BAYCOL®), fluvastatin (LESCOL®), atorvastatin (LIPITOR®), lovastatin (MEVACOR®), pravastatin (PRAVACHOL®), Simvastatin (ZOCOR®); estradiol; testosterone (optionally at elevated dosages; Stuve et al. Neurology 8:290-301 (2002)); hormone replacement therapy; treatment for symptoms secondary or related to MS (e.g., spasticity, incontinence, pain, fatigue); a TNF inhibitor; disease-modifying anti-rheumatic drug (DMARD); non-steroidal anti-inflammatory drug (NSAID); plasmapheresis; levothyroxine; cyclosporin A; somatastatin analogue; cytokine or cytokine receptor antagonist; anti-metabolite; immunosuppressive agent; rehabilitative surgery; radioiodine; thyroidectomy; another B-cell surface antagonist/antibody; etc.

In some embodiments, the second medicament is administered with one or more anti-CD20 antibody administrations, such combined administration includes co-administration, using separate formulations or a single pharmaceutical formulation, and consecutive administration in either order, wherein preferably there is a time period while both (or all) active agents simultaneously exert their biological activities.

In some embodiments, the anti-CD20 antibody is the only medicament administered to the patient to treat multiple sclerosis. In some embodiments, the anti-CD20 antibody is the only disease modifying therapy (DMT) administered to the patient to treat multiple sclerosis. For example, in some embodiments, the anti-CD20 antibody is administered in combination with one or more of: methylprednisolone (or equivalent); an antihistamine (e.g., diphenhydramine or equivalent); an analgesic (e.g., acetaminophen); and an antipyretic.

In some embodiments, the anti-CD20 antibody is not used concomitantly with a non-live vaccine. In some embodiments, the non-live vaccine is a tetanus toxoid-containing vaccine, a pneumococcal polysaccharide, a pneumococcal conjugate vaccine, or a seasonal inactivated influenza vaccine.

In some embodiments, administration of a live or live-attenuated vaccine occurs at least 4 weeks prior to the start of treatment with the anti-CD20 antibody.

### G. Side Effects

The present application in one aspect provides a method of treating multiple sclerosis in a patient comprising administering an effective amount of an anti-CD20 antibody subcutaneously at a dose of about 920 mg, wherein the patient does not develop significant side effect, substantial adverse drug reactions, or the treatment has resulted in a comparable or more favorable safety profile as compared to that after an intravenous administration of the anti-CD20 antibody at a dose of about 600 mg. In some embodiments, the patient does not have a more than about 20%, 18%, or 15% risk of developing a reaction (e.g., an injection reaction) that is of moderate severity. In some embodiments, the patient does not develop a serious injection reaction. In some embodiments, when the patient does develop an injection reaction, the patient has a risk of no more about 20%, 18%, or 15% of developing an injection reaction that is of moderate severity.

In some embodiments, the side effect comprises an infusion-related reaction (IRR). All CD20-depleting agents, including ocrelizumab, have been associated with acute IRRs. Symptoms associated with IRRs included, but are not limited to pruritus, rash, urticaria, erythema, throat irritation, oropharyngeal pain, dyspnea, pharyngeal or laryngeal edema, flushing, hypotension, pyrexia, fatigue, headache, dizziness, nausea, tachycardia, and anaphylaxis. In some embodiments, the methods described herein result in decreased incidence of IRRs as compared to methods involving an intravenous administration of the anti-CD20 antibody at a dose of about 600 mg.

In some embodiments, the side effect comprises an injection-site reaction (ISR). In some embodiments, the injection-site reaction may occur during any subcutaneous administration of an anti-CD20 antibody. In some embodiments, the injection-site reaction may comprise injection site erythema, pain, swelling, bruising, pruritus, hypersensitivity, and/or urticaria. In some embodiments, the patient is premedicated to reduce the risk of an injection-site reaction. In some embodiments, the methods described herein result in decreased incidence of ISRs as compared to methods involving an intravenous administration of the anti-CD20 antibody at a dose of about 600 mg.

In some embodiments, the side effect comprises an infection. In some embodiments, the infection may comprise a mild to moderate respiratory tract infection. In some embodiments, the infection may be an opportunistic infection. In some embodiments, the infection may be a non-disseminated herpes virus-associated infection. In some embodiments, the infection may be a hepatitis B virus infection. In some embodiments, the patient is less likely to develop an infection following subcutaneous administration of an anti-CD20 antibody compared to that after an intravenous administration of the anti-CD20 antibody at a dose of about 600 mg.

In some embodiments, the side-effect is a decrease in total immunoglobulins (Ig) over the controlled period (e.g., the first 24 weeks of the treatment) of treatment with an anti-CD20 antibody. In some embodiments, the decrease in total immunoglobulins is mainly driven by a reduction in IgM. In some embodiments, the proportion of patients with total immunoglobulins below the lower limit of normal (LLN) increases over time and with successive dosing with an anti-CD20 antibody. In some embodiments, the patient is less likely to develop a decrease in total immunoglobulins following subcutaneous administration of an anti-CD20 antibody compared to that after an intravenous administration of the anti-CD20 antibody at a dose of about 600 mg.

In some embodiments the side effect comprises a systemic injection reaction, progressive multifocal leukoencephalopathy, hypersensitivity reactions, malignancies, and/or neutropenia. In some embodiments, the patient experiences a lower risk or incidence of developing any of the side-effects described above or herein following subcutaneous administration of an anti-CD20 antibody compared to that after an intravenous administration of the anti-CD20 antibody at a dose of about 600 mg.

### H. Technical Effects

The present application provides methods that achieve non-inferior pharmacokinetic (PK) results as compared to the approved MS treatment via IV administration of ocrelizumab at a dose of 600 mg. In some embodiments, provided herein is a method of treating multiple sclerosis in a patient comprising administering an anti-CD20 antibody subcutaneously at a dosage of about 920 mg, optionally about every 6 months or 24 weeks for about 12, 18, or 24 months, wherein the serum ocrelizumab area under the concentration-time curve from day 1 to week 12 post subcutaneous administration of an anti-CD20 antibody described herein (AUC_{W1-12}) at a dose of about 920 mg is comparable to that post IV administration of the anti-CD20 antibody (e.g., ocrelizumab) at a dose of 600 mg. In some embodiments, the 25th percentile in the exposure (AUCt) distribution associated with the methods described herein is comparable to that associated with the IV administration of the anti-CD20 antibody (e.g., ocrelizumab) at a dose of 600 mg. In some cases, “comparable” described herein refers to when a value falls in a range of about 50% to about 200%, about 60% to about 170%, about 70% to about 150%, about 75% to about 135%, about 80% to about 120%, or 90% to about 110% of the comparator value or reference value.

### III. Compositions, Formulations, and Unit Dosages

Therapeutic formulations of the antibodies used in accordance with the present invention are prepared for storage by mixing an antibody having the desired degree of purity with optional pharmaceutically acceptable carriers, excipients or stabilizers (Remington's Pharmaceutical Sciences 16^{th} edition, Osol, A. Ed. (1980)), in the form of lyophilized formulations or aqueous solutions. Acceptable carriers, excipients, or stabilizers are nontoxic to recipients at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as TWEEN™, PLURONICS™ or polyethylene glycol (PEG).

Lyophilized formulations adapted for subcutaneous administration are described in U.S. Pat. No. 6,267,958 (Andya et al.). Such lyophilized formulations may be reconstituted with a suitable diluent to a high protein concentration and the reconstituted formulation may be administered subcutaneously to the mammal to be treated herein.

Crystalized forms of the antibody or antibody are also contemplated. See, for example, US 2002/0136719A1 (Shenoy et al.).

The formulation herein may also contain more than one active compound as necessary for the particular indication being treated, in some embodiments, those with complementary activities that do not adversely affect each other. For example, it may be desirable to further provide a cytotoxic agent; chemotherapeutic agent; immunosuppressive agent; cytokine; cytokine antagonist or antibody; growth factor; hormone; integrin; integrin antagonist or antibody (e.g. an LFA-1 antibody, or an alpha 4 integrin antibody such as natalizumab/TYSABRI®) available from Biogen Idec/Elan Pharmaceuticals, Inc.); interferon class drug such as IFN-beta-1a (REBIF® and AVONEX®) or IFN-beta-1b (BETASERON®); an oligopeptide such a glatiramer acetate (COPAXONE®); a cytotoxic agent such as mitoxantrone (NOVANTRONE®), methotrexate, cyclophosphamide, chlorambucil, or azathioprine; intravenous immunoglobulin (gamma globulin); lymphocyte-depleting drug (e.g., mitoxantrone, cyclophosphamide, Campath, anti-CD4, or cladribine); non-lymphocyte-depleting immunosuppressive drug (e.g., mycophenolate mofetil (MMF) or cyclosporine); cholesterol-lowering drug of the “statin” class; estradiol; testosterone; hormone replacement therapy; drug that treats symptoms secondary or related to MS (e.g., spasticity, incontinence, pain, fatigue); a TNF inhibitor; disease-modifying anti-rheumatic drug (DMARD); non-steroidal anti-inflammatory drug (NSAID); corticosteroid (e.g. methylprednisolone, prednisone, dexamethasone, or glucorticoid); levothyroxine; cyclosporin A; somatastatin analogue; cytokine antagonist; anti-metabolite; immunosuppressive agent; integrin antagonist or antibody (e.g. an LFA-1 antibody, such as efalizumab or an alpha 4 integrin antibody such as natalizumab); or another B-cell surface antagonist/antibody; etc. in the formulation. The type and effective amounts of such other agents depend, for example, on the amount of antibody present in the formulation, the type of multiple sclerosis being treated, and clinical parameters of the patients. These are generally used in the same dosages and with administration routes as used hereinbefore or about from 1 to 99% of the heretofore employed dosages.

The active ingredients may also be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions. Such techniques are disclosed in Remington's Pharmaceutical Sciences 16^{th} edition, Osol, A. Ed. (1980).

Sustained-release preparations may be prepared. Suitable examples of sustained-release preparations include semipermeable matrices of solid hydrophobic polymers containing the antibody, which matrices are in the form of shaped articles, e.g. films, or microcapsules. Examples of sustained-release matrices include polyesters, hydrogels (for example, poly(2-hydroxyethyl-methacrylate), or poly(vinylalcohol)), polylactides (U.S. Pat. No. 3,773,919), copolymers of L-glutamic acid and y ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), and poly-D-(−)-3-hydroxybutyric acid.

The formulations to be used for in vivo administration must be sterile. This is readily accomplished by filtration through sterile filtration membranes.

In some embodiments, the formulation comprises one or more of the group consisting of a sodium acetate, trehalose, methionine, polysorbate 20, and hyaluronidase. In some embodiments, the sodium acetate is a sodium acetate buffer, pH of about 5.3. Examples of formulations suitable for the administration of the anti-CD20 antibody are found in Andya et al., US2006/0088523, WO98/56418, and US20210054092A1 which are incorporated by reference in their entirety with respect to formulations.

In some embodiments, there is provided a pharmaceutical formulation of a pharmaceutically active anti-CD20 antibody suitable for subcutaneous administration comprising about 30 mg/ml to 350 mg/ml, for example about 30 mg/ml to 100 mg/ml (including about 30 mg/ml, about 50 mg/ml or about 100 mg/ml) anti-CD20 antibody described herein (e.g., ocrelizumab); about 1 to 100 mM of a buffering agent (e.g. sodium acetate) providing a pH of 5.5±2.0 (e.g. pH 5.3); about 15 to 250 mM of a stabilizer or a mixture of two or more stabilizers (including trehalose, e.g. about 8% trehalose dihydrate); about 0.01 to 0.1% (w/v) of a nonionic surfactant; and optionally an effective amount of at least one hyaluronidase enzyme (e.g. rhHUPH20), preferably in an amount from about 1,500 U/ml to about 12,000 U/ml.

In some embodiments, the formulation provided herein is a liquid single-dose or multidose formulation comprising the anti-CD20 antibody at about 30-50 mg/mL (e.g., about 40 mg/mL), about 15-25 mM (e.g., 20 mM) sodium acetate providing a pH of about 5 to about 5.6 (e.g., 5.3), about 220 mM to about 260 mM (e.g., 240 mM) trehalose, about 5 mM to 15 mM (e.g., 10 mM) methionine, about 0.05-0.07% (w/v) (e.g., 0.06%) polysorbate 20, and about 800-1200 U/ml (e.g., 1000 U/ml) of hyaluronidase, wherein the anti-CD20 antibody comprises a V_{H} domain comprising the amino acid sequence set forth in SEQ ID NO: 8, a V_{L} domain comprising the amino acid sequence set forth in SEQ ID NO: 7, and a human IgG1 constant region. In some embodiments, the anti-CD20 antibody is ocrelizumab. In some embodiments, the formulation is for subcutaneous administration. In some embodiments, the formulation has a minimum shelf life of two years storage at 2-8° C. In some embodiments, the formulation provided herein is a liquid formulation comprising the anti-CD20 antibody at about 35 to about 45 mg/mL (e.g., about 40 mg/mL), about 20 mM sodium acetate providing a pH of about 5 to about 5.6 (e.g., 5.3), about 190 mM to about 290 mM (e.g., 240 mM) trehalose, about 5 mM to 15 mM (e.g., 10 mM) methionine, about 0.04-0.08% (w/v) (e.g., 0.06%) polysorbate 20, and about 1000 U/ml of hyaluronidase, wherein the anti-CD20 antibody comprises a V_{H} domain comprising the amino acid sequence set forth in SEQ ID NO: 8, a V_{L} domain comprising the amino acid sequence set forth in SEQ ID NO: 7, and a human IgG1 constant region. In some embodiments, the anti-CD20 antibody is ocrelizumab. In some embodiments, the formulation is for subcutaneous administration. In some embodiments, the formulation has a minimum shelf life of two years storage at 2-8° C.

In some embodiments, there is provided a pharmaceutical formulation of a pharmaceutically active anti-CD20 antibody suitable for subcutaneous administration comprising an anti-CD20 antibody, sodium acetate, trehalose, methionine, polysorbate 20, and hyaluronidase, optionally wherein a) the concentration of the antibody versus the concentration of sodium acetate is about 35-45 mg/mL (e.g., about 40 mg/mL): 20 mM, b) the concentration of the antibody versus the concentration of trehalose is about 35-45 mg/ml (e.g., about 40 mg/mL): about 190 mM to about 290 mM (e.g., 240 mM), c) the concentration of the antibody versus the concentration of methionine is about 35-45 mg/mL (e.g., about 40 mg/mL): 5 mM to 15 mM (e.g., 10 mM), d) the concentration of the antibody versus the concentration of polysorbate 20 is about 35-45 mg/mL (e.g., about 40 mg/mL): 0.04-0.08% (w/v) (e.g., 0.06%), and/or e) the concentration of the antibody versus the concentration of hyaluronidase about 35-45 mg/mL (e.g., about 40 mg/mL): about 1000 U/ml. In some embodiments, the formulation comprises about 23,000 units of hyaluronidase. In some embodiments, the concentration of the anti-CD20 antibody, sodium acetate, trehalose, methionine, polysorbate 20, and hyaluronidase is 40 mg/mL: 20 mM: 240 mM: 10 mM: 0.06% (w/v): 1000 U/ml. In some embodiments, the anti-CD20 antibody comprises a V_{H} domain comprising the amino acid sequence set forth in SEQ ID NO: 8, a V_{L} domain comprising the amino acid sequence set forth in SEQ ID NO: 7, and a human IgG1 constant region. In some embodiments, the anti-CD20 antibody is ocrelizumab. In some embodiments, the formulation has a minimum shelf life of two years storage at 2-8° C.

In some embodiments, the anti-CD20 antibody is in a pharmaceutical formulation, wherein the pharmaceutical formulation comprises about 920 mg of the anti-CD20 antibody, 23,000 units of hyaluronidase, about 2.088 g of α,α-trehalose dihydrate, about 5.5 mg of glacial acetic acid, about 34.4 mg of L-methionine, about 13.8 mg of polysorbate 20, about 50.1 mg of sodium acetate trihydrate, and water for injection at a pH of about 5.3, wherein the anti-CD20 antibody comprises a V_{H} domain comprising the amino acid sequence set forth in SEQ ID NO: 8, a V_{L} domain comprising the amino acid sequence set forth in SEQ ID NO: 7, and a human IgG1 constant region. In some embodiments, the anti-CD20 antibody is ocrelizumab. In some embodiments, the anti-CD20 antibody is supplied in a 50 mL single-dose vial. In some embodiments, the formulation is for subcutaneous administration. In some embodiments, the formulation is stored at about 2-8° C. In some embodiments, the antibody formulation is stored at or above about 25° C. for no more than 12 hours. In some embodiments, each vial is ready-to-use for one subcutaneous injection. In some embodiments, contents of the vial are not diluted.

In some embodiments, recombinant humanized 2H7 anti-CD20 antibody (2H7.v16 as disclosed in WO 2006/084264, which is incorporated in its entirety by reference) was used for the composition or formulation described herein.

Lyophilized formulations adapted for subcutaneous administration are described in WO97/04801, which is incorporated by reference in its entirety. Such lyophilized formulations may be reconstituted with a suitable diluent to a high protein concentration and the reconstituted formulation may be administered subcutaneously to the mammal to be treated herein.

In some embodiments, the antibody in the formulation is stable at −20° C. for at least about 6 months, at least about 12 months, at least about 18 months, at least two years, at least three years, or at least four years. In some embodiments, the antibody in the formulation is stable at 2-8° C. for at least about 1, 2, 3, or 6 months. In some embodiments, after storage, the antibody retains at least about 60%, at least about 65%, at least about 70%, at least about 75%, at least about 80%, at least about 85%, at least about 90%, or at least about 95% of its biological activity (e.g., binding to the target, or therapeutic potency) exhibited before storage, i.e., at the time the pharmaceutical formulation was prepared. In some embodiments, the stability as

In some embodiments, the antibody in the formulation is stable at 2-8° C. for at least about 1, 2, 3, 4, 5, or 6 months.

In some embodiments, the antibody formulation is stored at or above about 25° C. for no more than 12 hours.

In some embodiments, the antibody formulation after storage does not have a) a change of more than about 20% or 17.5% increase in acidic region as assessed by ion-exchange high-performance liquid chromatography (IE-HPLC), b) a change of more than about 25%, 22% or 20% decrease in main peak as assessed by IE-HPLC, and/or c) a change of more than 3% or 2% increase in basic region as assessed by IE-HPLC, as compared to that prior to storage. In some embodiments, the antibody formulation after storage does not have a) a change of more than about 0.2% or 0.1% increase in the sum of high molecule weight forms (HWM) as assessed by size-exclusion ultra-high performance liquid chromatography (SE-UHPLC), and/or b) a change of more than 1%, 0.8%, 0.7%, or 0.6% decrease in main peak as assessed by SE-UHPLC, as compared to that prior to storage. In some embodiments, the antibody formulation after storage does not have a) a change of more than about 3%, 2.7%, or 2.5% increase in the sum of low molecule weight forms (LWM) as assessed by non-reduced capillary electrophoresis sodium dodecyl sulfate (NR CE-SDS), and/or b) a change of more than 3% decrease in main peak as assessed by NR CE-SDS, as compared to that prior to storage. In some embodiments, the antibody formulation after storage does not have a) a change of more than 0.7% or 0.6% increase in the HC-M257 oxidation as assessed by peptide map, b) a change of more than 0.1% increase in the HC-M433 oxidation as assessed by peptide map, and/or c) a change of more than 0.1% increase in the HC-W107 oxidation as assessed by peptide map, as compared to that prior to storage. In some embodiments, the antibody formulation after storage does not have a change of more than about 500 U/mL or 450 U/mL decrease in rHuPH20 activity. In some embodiments, the antibody formulation after storage does not have a change of more than 0.25 e4 U/mg decrease in potency by CDC Bioassay. In some embodiments, the antibody formulation is stored at about 2 to about 8° C. for at least about 1, 2, 3, or 6 months. In some embodiments, the antibody formulation is stored at about 25° C. for at least about 0.5, 1, 2, 3, or 6 months at 60% relative humidity.

In some embodiments, the antibody formulation has a better stability in size variants (such as HMWS by SE-UPLC and/or LMWS by NR CE-SES) than a reference antibody formulation (e.g., an IV antibody formulation) of said anti-CD20 antibody after exposure to a stressed condition. In some embodiments, the reference antibody formulation comprises 30 mg/mL anti-CD20 antibody, 20 mM sodium acetate, 106 mM trehalose, 0.2 mg/mL PS20 and has a pH 5.3. In some embodiments, the stressed condition comprises exposing the antibody formulation at about 40° C. for about 4 weeks at 75% relative humidity.

In some embodiments, the antibody formulation does not have a change of more than about 0.6% decrease in the main peak as measured by SE-UHPLC after exposure to a stressed condition. In some embodiments, the antibody formulation has a comparable change in the percentage (e.g., about 0.6% decrease) in the main peak as measured by SE-UHPLC after exposure to a stressed condition, as compared to that of a reference antibody formulation exposed to the same stressed condition. In some embodiments, the antibody formulation does not have a change of the percentage high molecular weight species (HMWS) as measured by SE-UHPLC after exposure to a stressed condition. In some embodiments, the antibody formulation exhibited better stability characterized by a smaller change in the percentage HMWS as measured by SE-UHPLC than a reference antibody after exposed to a similar stressed condition (no change for the antibody formulation vs. an increase of 0.1% for the reference antibody formulation). In some embodiments, the antibody formulation does not have a change of more than 0.4% increase in low molecular weight species (LMWS) as measured by SE-UHPLC after exposure to a stressed condition. In some embodiments, the antibody formulation has a comparable change in the percentage (e.g., about 0.4% increase) in low molecular weight species (LMWS) as measured by SE-UHPLC after exposure to a stressed condition, as compared to that of a reference antibody formulation exposed to the same stressed condition. In some embodiments, the antibody formulation has a comparable change in the percentage (e.g., about 0.02%) in the main peak determined by linear regression per day as measured by SE-UHPLC after exposure to a stressed condition, as compared to that of a reference antibody formulation exposed to the same stressed condition. In some embodiments, the antibody formulation does not have a change of more than 0.003% per day in the HMWS peak determined by linear regression as measured by SE-UHPLC after exposure to a stressed condition. In some embodiments, the antibody formulation exhibited better stability characterized by a smaller change in the percentage per day in the HMWS peak determined by linear regression as measured by SE-UHPLC after exposure to a stressed condition, as compared to that of a reference antibody formulation exposed to the same stressed condition (0.003% change per day for the antibody formulation vs. 0.006% per day for the reference antibody formulation). In some embodiments, the reference antibody formulation comprises 30 mg/mL anti-CD20 antibody, 20 mM sodium acetate, 106 mM trehalose, 0.2 mg/mL PS20 and has a pH 5.3. In some embodiments, the stressed condition comprises exposing the antibody formulation at about 40° C. for about 4 weeks at 75% relative humidity.

In some embodiments, the antibody formulation does not have a change of more than about 2.5% decrease (e.g., about 2.1% decrease) in the main peak as measured by non-reduced CE-SDS after exposure to a stressed condition. In some embodiments, the antibody formulation exhibited better stability characterized by a smaller change in the percentage in the main peak as measured by non-reduced CE-SDS after exposure to a stressed condition, as compared to that of a reference antibody formulation exposed to the same stressed condition (e.g., 2.1% decrease for the antibody formulation vs. 2.7% decrease for the reference antibody formulation). In some embodiments, the antibody formulation does not have a change of more than about 2.5% (e.g., 2.2%) increase in the sum of the pre-peaks as measured by non-reduced CE-SDS after exposure to a stressed condition. In some embodiments, the antibody formulation exhibited better stability characterized by a smaller change in the percentage in the sum of the pre-peaks as measured by non-reduced CE-SDS after exposure to a stressed condition, as compared to that of a reference antibody formulation exposed to the same stressed condition (e.g., 2.2% increase for the antibody formulation vs. 2.5% increase for the reference antibody formulation). In some embodiments, the antibody formulation does not have a change of more than about-0.1% per day in the main peak determined by linear regression as measured by non-reduced CE-SDS after exposure to a stressed condition. In some embodiments, the antibody formulation has a comparable change in the percentage per day in the main peak determined by linear regression as measured by non-reduced CE-SDS after exposure to a stressed condition, as compared to that of a reference antibody formulation exposed to the same stressed condition. In some embodiments, the antibody formulation does not have a change of more than about 0.1% per day the sum of the pre-peak determined by linear regression as measured by non-reduced CE-SDS after exposure to a stressed condition. In some embodiments, the antibody formulation has a comparable change in the percentage per day in the sum of the pre-peak determined by linear regression as measured by non-reduced CE-SDS after exposure to a stressed condition, as compared to that of a reference antibody formulation exposed to the same stressed condition. In some embodiments, the antibody formulation has a comparable electrophoretic profile as measured by non-reduced CE-SDS after exposure to a stressed condition, as compared to that of a reference antibody formulation exposed to the same stressed condition. In some embodiments, the reference antibody formulation comprises 30 mg/mL anti-CD20 antibody, 20 mM sodium acetate, 106 mM trehalose, 0.2 mg/mL PS20 and has a pH 5.3. In some embodiments, the stressed condition comprises exposing the antibody formulation at about 40° C. for about 4 weeks at 75% relative humidity.

In some embodiments, the antibody formulation does not have a change of more than about 25% (e.g., about a decrease of 23%) in the main peak as measured by IE-HPLC after exposure to a stressed condition. In some embodiments, the antibody formulation has a comparable change in the percentage in the main peak as measured by IE-HPLC after exposure to a stressed condition, as compared to that of a reference antibody formulation exposed to the same stressed condition (e.g., a decrease of 22.9% for the antibody formulation vs. a decrease of 23.5% for the reference antibody formulation). In some embodiments, the antibody formulation does not have a change of more than about 25% (e.g., about an increase of about 22%) in the acidic region as measured by IE-HPLC after exposure to a stressed condition. In some embodiments, the antibody formulation has a comparable change in the percentage in the acidic region as measured by IE-HPLC after exposure to a stressed condition, as compared to that of a reference antibody formulation exposed to the same stressed condition (e.g., an increase of 22.2% for the antibody formulation vs. an increase of 22.2% for the reference antibody formulation). In some embodiments, the antibody formulation does not have a change of more than about-1% (e.g., −0.8%) per day in the main peak determined by linear regression as measured by IE-HPLC after exposure to a stressed condition. In some embodiments, the antibody formulation has a comparable change in the percentage in the main peak determined by linear regression as measured by IE-HPLC after exposure to a stressed condition, as compared to that of a reference antibody formulation exposed to the same stressed condition (e.g., −0.8% vs. −0.8%). In some embodiments, the antibody formulation has a comparable chromatographic profile, as compared to that of a reference antibody formulation exposed to the same stressed condition. In some embodiments, the reference antibody formulation comprises 30 mg/mL anti-CD20 antibody, 20 mM sodium acetate, 106 mM trehalose, 0.2 mg/mL PS20 and has a pH 5.3. In some embodiments, the stressed condition comprises exposing the antibody formulation at about 40° C. for about 4 weeks at 75% relative humidity.

In some embodiments, the antibody formulation does not have a change of the potency per CDC bioassay more than about 0.2×10⁴ U/mg decrease after exposure to a stressed condition. In some embodiments, the antibody formulation has a comparable change in the potency per CDC bioassay after exposure to a stressed condition, as compared to that of a reference antibody formulation exposed to the same stressed condition.

In some embodiments, the antibody formulation has a shelf-life for at least 12 months, 24 months, or 36 months.

### Unit Dosages

The present application in another aspect provides unit dosage forms of an anti-CD20 antibody comprising a sealed vial containing a quantity of an anti-CD20 antibody suitable for subcutaneous administration to a patient, wherein said quantity is sufficient to deliver a dose of about 920 mg. In some embodiments, the anti-CD20 antibody comprises a V_{H} domain comprising the amino acid sequence set forth in SEQ ID NO: 8, a V_{L} domain comprising the amino acid sequence set forth in SEQ ID NO: 7, and a human IgG1 constant region. In some embodiments, the anti-CD20 antibody is comprised in a liquid formulation at the concentration of about 30-50 mg/ml (e.g., 40 mg/ml). In some embodiments, the anti-CD20 antibody is comprised in a liquid formulation at the concentration of about 40 mg/ml.

In some embodiments, said formulation further comprises a buffering agent, a stabilizer and surfactant, optionally wherein the formulation further comprises a hyaluronidase.

In some embodiments, the buffering agent comprises sodium acetate, optionally wherein the formulation comprises about 15-25 mM (e.g., 20 mM) sodium acetate providing a pH of about 5-5.6 (e.g., pH of 5.3).

In some embodiments, the stabilizer comprises trehalose and/or methionine, optionally wherein the formulation comprises a) about 200 mM to about 300 mM (e.g., about 240 mM) trehalose and/or b) about 8-12 mM methionine (e.g., 10 mM) methionine.

In some embodiments, the surfactant comprises polysorbate 20, and optionally wherein the formulation comprises about 0.04-0.08% (e.g., 0.06%) (w/v) polysorbate 20.

In some embodiments, the hyaluronidase comprises a recombinant human hyaluronidase, optionally the recombinant human hyaluronidase is rHuPH20, further optionally the formulation comprises about 800-1200 U/ml (e.g., 1000 U/ml) rHuPH20. In some embodiments, the formulation comprises about 23,000 units of hyaluronidase.

### IV. Articles of Manufacture

The invention further provides articles of manufacture or kits (such as kits-of parts) containing materials useful for the treatment of multiple sclerosis (e.g., relapsing multiple sclerosis or primary progressive multiple sclerosis) described herein. In some embodiments, the article of manufacture comprising, packaged together, a pharmaceutical composition comprising an anti-CD20 antibody and a pharmaceutically acceptable carrier and a label denoting that the anti-CD20 antibody or pharmaceutical composition is indicated for treating patients with multiple sclerosis (e.g., RMS or PPMS) according to a method described herein.

In some embodiments, the article of manufacture or kit comprises, packaged together, a pharmaceutical composition comprising an anti-CD20 antibody and a pharmaceutically acceptable carrier and a label denoting the anti-CD20 antibody or pharmaceutical composition is indicated for treating patients with multiple sclerosis and suppresses disability progression in patients having multiple sclerosis. In some embodiments, the article of manufacture or kit comprises, packaged together, a pharmaceutical composition comprising an anti-CD20 antibody and a pharmaceutically acceptable carrier and a label denoting the anti-CD20 antibody or pharmaceutical composition is indicated for treating patients with multiple sclerosis (e.g., RMS or PPMS). In some embodiments, the label provides instructions for administering an effective amount of an anti-CD20 antibody subcutaneously to the patient at a dose of about 920 mg of the anti-CD20 antibody. In some embodiments, the label states that the anti-CD20 antibody is administered subcutaneously every 6 months for about 12, 18, or 24 months. In some embodiments, the label states that the anti-CD20 antibody is administered subcutaneously every 24 weeks for about 12, 18, or 24 months. In some embodiments, the anti-CD20 antibody comprises a V_{H} domain comprising the amino acid set forth in SEQ ID NO: 8, a V_{L} domain comprising the amino acid sequence set forth in SEQ ID NO: 7, and a human IgG1 constant region. In some embodiments, the anti-CD20 antibody comprises a light chain comprising the amino acid sequence of SEQ ID NO: 9 and a heavy chain comprising the amino acid sequence of SEQ ID NO: 11.

In certain embodiments, the article of manufacture or kit comprises a container and a label or package insert on or associated with the container. Suitable containers include, for example, bottles, vials, syringes (e.g., prefilled syringes), etc. The containers may be formed from a variety of materials such as glass or plastic. The container holds or contains a composition that is effective for treating the multiple sclerosis and may have a sterile access port (for example the container may be an intravenous solution bag or a vial having a stopper pierceable by a hypodermic injection needle). At least one active agent in the composition is the antibody. In some embodiments, the container comprises about 920 mgs of the anti-CD20 antibody.

In some embodiments, the article of manufacture or kit comprises a device suitable for subcutaneous administration of a volume of at least 20 mL (e.g., about 23 mL, or about 24 mL) comprising a needle. In some embodiments, the article of manufacture or kit comprises a device suitable for subcutaneous administration of a volume of about 23 mL comprising a needle. In some embodiments, the device is an on-body injector. In some embodiments, the device comprises an infusion pump. In some embodiments, the device comprises a CRONO ambulatory infusion pump. In some embodiments, the device comprises a Lapas patch pump. In some embodiments, the device comprises an enFuse on-body platform. In some embodiments, the device comprises a wearable injector or wearable injection device. In some embodiments, the device comprises a DrugDeliverySystem. See e.g., Badkar et al., Drug Des Devel Ther. 2021 Jan. 13; 15:159-170.

The label or package insert indicates that the composition is used for treating multiple sclerosis in a patient suffering therefrom with specific guidance regarding dosing amounts and intervals of antibody and any other drug being provided. The article of manufacture may further comprise a second container comprising a pharmaceutically acceptable diluent buffer, such as bacteriostatic water for injection (BWFI), phosphate-buffered saline, Ringer's solution and dextrose solution. The article of manufacture may further include other materials desirable from a commercial and user standpoint, including other buffers, diluents, filters, needles, and syringes.

Optionally, the article of manufacture or kit provided herein further comprises a container comprising an agent other than the antibody for treatment and further comprising instructions on treating the patient with such agent, such agent preferably being a chemotherapeutic agent or immunosuppressive agent, interferon class drug such as IFN-beta-1a (REBIF® and AVONEX®) or IFN-beta-1b (BETASERON®); an oligopeptide such a glatiramer acetate (COPAXONE®); a cytotoxic agent such as mitoxantrone (NOVANTRONE®), methotrexate, cyclophosphamide, chlorambucil, or azathioprine; intravenous immunoglobulin (gamma globulin); lymphocyte-depleting drug (e.g., mitoxantrone, cyclophosphamide, Campath, anti-CD4, or cladribine); non-lymphocyte-depleting immunosuppressive drug (e.g., mycophenolate mofetil (MMF) or cyclosporine); cholesterol-lowering drug of the “statin” class; estradiol; hormone replacement therapy; drug that treats symptoms secondary or related to MS (e.g., spasticity, incontinence, pain, fatigue); a TNF inhibitor; disease-modifying anti-rheumatic drug (DMARD); non-steroidal anti-inflammatory drug (NSAID); corticosteroid (e.g. methylprednisolone, prednisone, dexamethasone, or glucorticoid); levothyroxine; cyclosporin A; somatastatin analogue; cytokine or cytokine receptor antagonist; anti-metabolite; immunosuppressive agent; integrin antagonist or antibody (e.g. an LFA-1 antibody, such as efalizumab or an alpha 4 integrin antibody such as natalizumab); and another B-cell surface marker antibody; etc.

In some embodiments, the article of manufacture comprises a single-use vial containing a quantity of an anti-CD20 antibody suitable for subcutaneous administration to a patient, wherein said quantity is sufficient to deliver a dose of about 920 mg. In some embodiments, the anti-CD20 antibody comprises a V_{H} domain comprising the amino acid sequence set forth in SEQ ID NO: 8, a V_{L} domain comprising the amino acid sequence set forth in SEQ ID NO: 7, and a human IgG1 constant region. In some embodiments, the anti-CD20 antibody is ocrelizumab.

### EXAMPLES

The following examples are included for illustrative purposes only and are not intended to limit the scope of the present disclosure.

### Example 1: The Ocrelizumab SC Drug Product as a Liquid Formulation for Subcutaneous Administration of Ocrelizumab Used in Clinical Trials (See Examples 2 and 3 Below)

The drug product is composed of 40 mg/mL ocrelizumab and 1000 U/mL rHuPH20 (standard formulation) in 20 mM sodium acetate, 240 mM trehalose, 10 mM L-methionine, 0.06% (w/v) polysorbate 20 (HP grade used in this study) at pH 5.3.

### Part A. Robustness of the Ocrelizumab SC Formulation

The purpose of this study was to determine the robustness of the ocrelizumab (Ocrevus) subcutaneous (SC) (referred herein as ocrelizumab SC) drug product (DP) formulation.

Table 1 summarizes the formulation components to be studied as part of the ocrelizumab SC DP formulation robustness study, along with their composition ranges and the study range for each formulation parameter. There were four ocrelizumab SC formulations prepared in this study per Table 2. These formulations were designed to assess the DP formulation robustness and to characterize the stability of ocrelizumab SC within the studied range of parameters.

TABLE 1
Ocrelizumab SC Formulation Component Target and Ranges

| | | Target | | |
|---|---|---|---|---|
| Formulation Components | | Formulation | Study Range | |
| Buffer | Sodium Acetate (mM) | 20 | 20 | |
| pH | — | 5.3 | 5.0 | 5.6 |
| Concentration | Protein (mg/mL) | 40 | 36 | 44 |
| Tonicifier | Trehalose (mM) | 240 | 190 | 290 |
| Surfactant | Polysorbate 20 (“PS20”) | 0.6 | 0.4 | 0.8 |
| | (mg/mL) | | | |
| Stabilizer | Methionine (mM) | 10 | 5 | 15 |
| Permeation | rHuPH20 (U/mL) | 1000 | 1000 | |
| Enhancer | | | | |

TABLE 2
Study Design to Determine Robustness of Ocrelizumab SC DP Formulation

| | | | | Sodium | | | | |
|---|---|---|---|---|---|---|---|---|
| | | | Protein | Acetate | Trehalose | Methionine | PS20 | rHuPH20 |
| Formulation | Pattern | pH | (mg/mL) | (mM) | (mM) | (mM) | (mg/mL) | (U/mL) |
| F1 | 0000000 | 5.3 | 40 | 20 | 240 | 10 | 0.6 | 1000 |
| F2 | −+0−−−0 | 5.0 | 44 | 20 | 190 | 5 | 0.4 | 1000 |
| F3 | +−0+++0 | 5.6 | 36 | 20 | 290 | 15 | 0.8 | 1000 |
| F4 | 0000000 | 5.3 | 40 | 20 | 240 | 10 | 0.6 | 1000 |

### Results

### 1. Stability at 2-8° C.

Results for the four DP formulations studied at recommended storage conditions (2-8° C.) for up to six months are shown in Table 3 and Table 4. Table 5 summarizes the degradation rates at 2-8° C. for up to six months. Little to no difference were observed between F2 and F3 and the target formulations (F1 and F4) in all product quality attributes tested. Plots of high molecular weight (HMW) forms and main peak by size-exclusion ultra-high performance liquid chromatography (SE-UHPLC), sum of low molecular weight (LMW) forms and main peak by size-exclusion ultra-high performance liquid chromatography (NR CE-SDS), acidic region, basic region and main peak by size-exclusion ultra-high performance liquid chromatography (IE HPLC), PS20, and rHuPH20 activity were measured and some of the data were summarized in Table 9.

Variation in the formulation ranges resulted in stability patterns that are similar to the target formulation up to the current available stability data of 6 months at 2-8° C. (the study continues for up to 36 months). All formulations shall meet the end of shelf life specification after 24 months of storage at 2-8° C.

TABLE 3
Stability Data for Ocrelizumab SC DP Robustness Formulations (F1 and F2) Stored at 2-8° C.

| | | | | | | | | | | Subvisible Particles | | | |
|---|---|---|---|---|---|---|---|---|---|---|---|---|---|
| | | | | | | | | | | (Cumulative | | | |
| | | | | | Osmolality | L- | Polysorbate | Protein | | Counts/Container) | | | |
| | Time | | Clarity/ | | (mOsmol/ | Met | 20 | Content | Visible | ≥2 | ≥5 | ≥10 | ≥25 |
| Formulation | (months) | Color | Opalescence | pH | kg) | (mM) | (mg/mL) | (mg/mL) | Particulate | μm | μm | μm | μm |
| F1 | 0 | ≤B7 | ≤Ref I | 5.2 | 334 | 12.6 | 0.64 | 39.6 | PFVP | 1963 | 353 | 62 | 0 |
| (Target) | 1 | NT | NT | NT | NT | NT | 0.61 | NT | PFVP | 2009 | 376 | 31 | 8 |
| | 3 | NT | NT | NT | NT | NT | 0.66 | NT | PFVP | 1472 | 376 | 69 | 0 |
| | 6 | ≤B7 | ≤Ref I | 5.4 | NT | NT | 0.64 | 39.6 | PFVP | 3228 | 460 | 31 | 0 |
| F2 | 0 | ≤B7 | ≤Ref I | 4.9 | NT | 6.6 | 0.45 | 43.8 | PFVP | 1434 | 261 | 23 | 8 |
| | 1 | NT | NT | NT | NT | NT | 0.43 | NT | PFVP | 1879 | 414 | 46 | 0 |
| | 3 | NT | NT | NT | NT | NT | 0.47 | NT | PFVP | 1288 | 299 | 31 | 0 |
| | 6 | ≤B7 | ≤Ref I | 5.0 | NT | NT | 0.44 | 44.7 | PFVP | 2017 | 345 | 39 | 8 |

| | | | | Non-Reduced | | | | | | | | | |
|---|---|---|---|---|---|---|---|---|---|---|---|---|---|
| | | SE-UHPLC | | CE-SDS | | | | | | | | | |
| | | Sum | | Sum | | | | | | | | | |
| | | of | | of | | | | | | | | Potency | |
| | | HMW | | LMW | | IE-HPLC | | | Peptide Map | | | by | |
| | | For | Main | For | Main | Acidic | Main | Basic | HC- | HC- | HC- | CDC | |
| | | ms | Peak | ms | Peak | Region | Peak | Region | M257 | M433 | W107 | Bioassay | rHuPH20 |
| | Time | (area | (area | (% | (% | (area | (area | (area | Oxidation | Oxidation | Oxidation | (e4 U/ | Activity |
| Formulation | (months) | %) | %) | CPA) | CPA) | %) | %) | %) | (%) | (%) | (%) | mg) | (U/mL) |
| F1 | 0 | 0.7 | 99.3 | 2.3 | 96.9 | 27.2 | 67.6 | 5.2 | 2.0 | 0.8 | 0.1 | 0.98 | 1071 |
| (Target) | 1 | 0.7 | 99.2 | 2.4 | 96.0 | 26.6 | 67.8 | 5.6 | 1.9 | 0.8 | 0.1 | NT | 1017 |
| | 3 | 0.7 | 99.2 | 2.9 | 95.8 | 26.6 | 67.9 | 5.4 | 2.1 | 0.8 | 0.1 | NT | 1065 |
| | 6 | 0.8 | 99.1 | 2.5 | 96.6 | 25.8 | 67.7 | 6.5 | 2.2 | 0.9 | 0.2 | 1.01 | 1067 |
| F2 | 0 | 0.7 | 99.3 | 2.3 | 96.8 | 27.2 | 67.8 | 5.0 | 2.3 | 1.0 | 0.1 | 1.04 | 1007 |
| | 1 | 0.7 | 99.3 | 2.4 | 96.0 | 26.8 | 67.7 | 5.5 | 2.1 | 0.9 | 0.2 | NT | 1003 |
| | 3 | 0.7 | 99.2 | 3.2 | 95.5 | 26.9 | 67.8 | 5.4 | 2.3 | 1.0 | 0.2 | NT | 1025 |
| | 6 | 0.7 | 99.1 | 2.8 | 96.2 | 26.2 | 67.6 | 6.2 | 2.8 | 1.1 | 0.2 | 0.97 | 1032 |

Note:
Values are rounded; therefore, the totals may not equal 100%. NT = Not Tested.

TABLE 4
Stability Data for Ocrelizumab SC DP Robustness Formulations (F3 and F4) Stored at 2-8° C.

| | | | | | | | | | | Subvisible Particles | | | |
|---|---|---|---|---|---|---|---|---|---|---|---|---|---|
| | | | | | | | | | | (Cumulative | | | |
| | | | | | Osmolality | L- | Polysorbate | Protein | | Counts/Container) | | | |
| | Time | | Clarity/ | | (mOsmol/ | Met | 20 | Content | Visible | ≥2 | ≥5 | ≥10 | ≥25 |
| Formulation | (months) | Color | Opalescence | pH | kg) | (mM) | (mg/mL) | (mg/mL) | Particulate | μm | μm | μm | μm |
| F3 | 0 | ≤B7 | ≤Ref I | 5.5 | 401 | 19.4 | 0.86 | 36.4 | PFVP | 1718 | 368 | 31 | 0 |
| | 1 | NT | NT | NT | NT | NT | 0.85 | NT | PFVP | 1848 | 292 | 46 | 0 |
| | 3 | NT | NT | NT | NT | NT | 0.84 | NT | PFVP | 1457 | 207 | 39 | 0 |
| | 6 | ≤B7 | ≤Ref I | 5.6 | NT | NT | 0.81 | 36.6 | PFVP | 959 | 177 | 16 | 16 |
| F4 | 0 | ≤B7 | ≤Ref I | 5.3 | 334 | NA | 0.67 | 39.9 | PFVP | 859 | 184 | 39 | 0 |
| (Target) | 1 | NT | NT | NT | NA | NA | 0.63 | NT | PFVP | 1242 | 146 | 8 | 0 |
| | 3 | NT | NT | NT | NA | NA | 0.63 | NT | PFVP | 2316 | 483 | 62 | 0 |
| | 6 | ≤B7 | ≤Ref I | 5.4 | NA | NA | 0.63 | 39.7 | PFVP | 2530 | 736 | 177 | 46 |

| | | | | Non-Reduced | | | | | | | | | |
|---|---|---|---|---|---|---|---|---|---|---|---|---|---|
| | | SE-UHPLC | | CE-SDS | | | | | | | | | |
| | | Sum | | Sum | | | | | | | | | |
| | | of | | of | | | | | | | | Potency | |
| | | HMW | | LMW | | IE-HPLC | | | Peptide Map | | | by | |
| | | For | Main | For | Main | Acidic | Main | Basic | HC- | HC- | HC- | CDC | |
| | | ms | Peak | ms | Peak | Region | Peak | Region | M257 | M433 | W107 | Bioassay | rHuPH20 |
| | Time | (area | (area | (% | (% | (area | (area | (area | Oxidation | Oxidation | Oxidation | (e4 U/ | Activity |
| Formulation | (months) | %) | %) | CPA) | CPA) | %) | %) | %) | (%) | (%) | (%) | mg) | (U/mL) |
| F3 | 0 | 0.7 | 99.2 | 2.3 | 96.8 | 27.4 | 67.6 | 5.1 | 2.3 | 1.1 | 0.2 | 1.03 | 1029 |
| | 1 | 0.7 | 99.2 | 2.5 | 95.9 | 26.7 | 67.9 | 5.4 | 2.1 | 0.9 | 0.1 | NT | 1016 |
| | 3 | 0.7 | 99.2 | 2.7 | 95.9 | 27.1 | 67.7 | 5.2 | 2.3 | 1.0 | 0.2 | NT | 1102 |
| | 6 | 0.8 | 99.1 | 2.6 | 96.5 | 26.6 | 67.8 | 5.6 | 3.1 | 1.1 | 0.2 | 1.01 | 1067 |
| F4 | 0 | 0.7 | 99.3 | 2.3 | 96.7 | 27.5 | 67.4 | 5.1 | 2.1 | 0.9 | 0.1 | 1.08 | 1019 |
| (Target) | 1 | 0.7 | 99.2 | 2.4 | 96.0 | 26.7 | 68.1 | 5.3 | 2.0 | 0.8 | 0.1 | NT | 1015 |
| | 3 | 0.7 | 99.2 | 2.6 | 96.2 | 26.7 | 68.0 | 5.3 | 2.2 | 0.9 | 0.1 | NT | 1039 |
| | 6 | 0.8 | 99.1 | 2.8 | 96.3 | 26.3 | 67.8 | 5.9 | 2.4 | 0.9 | 0.2 | 1.04 | 1038 |

Note:
Values are rounded; therefore, the totals may not equal 100%.

TABLE 5
Degradation Rate for Ocrelizumab SC DP Robustness Formulations at 2-8° C.

| | | | Non-Reduced | | | | | | |
|---|---|---|---|---|---|---|---|---|---|
| | SE-UHPLC | | CE-SDS | | | | | Polysorbate | rHuPH20 |
| | (area % | | (% CPA | | | | | 20 | Activity |
| | change/day) | | change/day) | | IE-HPLC | | | (mg/mL | (U/mL |
| | Sum of | | Sum of | | (area % change/day) | | | change | change |
| | HMW | Main | LMW | Main | Acidic | Main | Basic | per | per |
| Formulation | Forms | Peak | Forms | Peak | Region | Peak | Region | day)^{a} | day)^{a} |
| F1 | 0.0005 | −0.0007 | 0.0012 | −0.0004 | −0.0066 | 0.0002 | 0.0064 | 0.0001 | 0.1 |
| (Target) | | | | | | | | | |
| F2 | 0.0005 | −0.0008 | 0.0034 | −0.0024 | −0.0044 | −0.0011 | 0.0056 | 0.0001 | 0.2 |
| F3 | 0.0005 | −0.0009 | 0.0018 | 0.0000 | −0.0026 | 0.0007 | 0.0019 | −0.0002 | 0.3 |
| F4 | 0.0004 | −0.0008 | 0.0025 | −0.0011 | −0.0052 | 0.0011 | 0.0041 | −0.0002 | 0.1 |
| (Target) | | | | | | | | | |

^{a}Little or no changes were observed at 2-8° C.

### 2. Stability at 25° C./60% Relative Humidity (RH)

Results for the four DP formulations studied at accelerated storage conditions (25° C./60% RH) for up to six months (final timepoint) are shown in Table 6 and Table 7.

Table 8 summarizes the degradation rate of ocrelizumab SC DP robustness formulations at 25° C./60% RH.

Changes were observed for all four formulations on the ocrelizumab product quality up to 6 months at 25° C. and summarized in Table 9:

As expected, pH has the biggest effect on ocrelizumab SC drug product stability. Increase in the formulation pH from 5.3 (F2) to 5.8 (F3):

Decreases the loss of rHuPH20 activity.

Increases the formation of the acidic region and loss of main peak.

The changes in acidic variants are associated with asparagine deamidation in CDRs (primarily at HC Asn104) and non-CDRs (primarily at HC Asn330 and HC Asn389/Asn394), where the amide functional group of an asparagine residue is hydrolyzed to form aspartic acid, iso-aspartic acid, or corresponding succinimide intermediate. The nature of the modification causes deamidated variants to partition into the IE-HPLC acidic region.

The changes in rHuPH20 activity and charge variants at the pH edges over the allotted time at 25° C. (less than 8 days for the drug product) are acceptable and results in sufficiently similar product quality (e.g., meet the end-of-shelf life acceptance criteria together with storage at 2-8° C.) (see degradation rates in Table 8).

Plots of HMW forms and main peak by SE-UHPLC, sum of LMW forms and main peak by NR CE-SDS, acidic region, basic region, and main peak by IE-HPLC, PS20, and rHuPH20 activity were measured and some of the data were summarized in Table 9.

Little to no difference were observed between F2 and F3 and the target formulations (F1 and F4) in all other product quality attributes.

TABLE 6
Stability Data for Ocrelizumab SC DP Robustness Formulations (F1 and F2) Stored at 25° C./60% RH

| | | | | | | | | | | Subvisible Particles | | | |
|---|---|---|---|---|---|---|---|---|---|---|---|---|---|
| | | | | | | | | | | (Cumulative | | | |
| | | | | | Osmolality | L- | Polysorbate | Protein | | Counts/Container) | | | |
| | Time | | Clarity/ | | (mOsmol/ | Met | 20 | Content | Visible | ≥2 | ≥5 | ≥10 | ≥25 |
| Formulation | (months) | Color | Opalescence | pH | kg) | (mM) | (mg/mL) | (mg/mL) | Particulate | μm | μm | μm | μm |
| F1 | 0 | ≤B7 | ≤Ref I | 5.2 | 334 | 12.6 | 0.64 | 39.6 | PFVP | 1963 | 353 | 62 | 0 |
| (Target) | 0.5 | NT | NT | NT | NT | NT | 0.64 | NT | PFVP | 2891 | 560 | 69 | 0 |
| | 1 | NT | NT | NT | NT | NT | 0.61 | NT | PFVP | 2461 | 430 | 85 | 0 |
| | 3 | NT | NT | NT | NT | NT | 0.62 | NT | PFVP | 2116 | 437 | 77 | 0 |
| | 6 | ≤B7 | ≤Ref I | 5.4 | NT | NT | 0.64 | 39.8 | PFVP | 4784 | 1564 | 514 | 69 |
| F2 | 0 | ≤B7 | ≤Ref I | 4.9 | 271 | 6.6 | 0.45 | 43.8 | PFVP | 1434 | 261 | 23 | 8 |
| | 0.5 | NT | NT | NT | NT | NT | 0.46 | NT | PFVP | 1710 | 292 | 23 | 0 |
| | 1 | NT | NT | NT | NT | NT | 0.42 | NT | PFVP | 2224 | 476 | 69 | 0 |
| | 3 | NT | NT | NT | NT | NT | 0.45 | NT | PFVP | 1902 | 468 | 54 | 0 |
| | 6 | ≤B7 | ≤Ref I | 5.0 | NT | NT | 0.45 | 44.4 | PFVP | 1794 | 437 | 23 | 8 |

| | | | | Non-Reduced | | | | | | | | | |
|---|---|---|---|---|---|---|---|---|---|---|---|---|---|
| | | SE-HPLC | | CE-SDS | | | | | | | | | |
| | | Sum | | Sum | | | | | | | | | |
| | | of | | of | | | | | | | | Potency | |
| | | HMW | | LMW | | IE-HPLC | | | Peptide Map | | | by | |
| | | For | Main | For | Main | Acidic | Main | Basic | HC- | HC- | HC- | CDC | |
| | | ms | Peak | ms | Peak | Region | Peak | Region | M257 | M433 | W107 | Bioassay | rHuPH20 |
| | Time | (area | (area | (% | (% | (area | (area | (area | Oxidation | Oxidation | Oxidation | (e4 U/ | Activity |
| Formulation | (months) | %) | %) | CPA) | CPA) | %) | %) | %) | (%) | (%) | (%) | mg) | (U/mL) |
| F1 | 0 | 0.7 | 99.3 | 2.3 | 96.9 | 27.2 | 67.6 | 5.2 | 2.0 | 0.8 | 0.1 | 0.98 | 1071 |
| (Target) | 0.5 | 0.7 | 99.2 | 2.4 | 96.4 | 28.1 | 66.2 | 5.7 | 2.0 | 0.9 | 0.1 | NT | 1019 |
| | 1 | 0.7 | 99.1 | 2.8 | 95.5 | 29.1 | 64.8 | 6.0 | 1.9 | 0.8 | 0.2 | NT | 997 |
| | 3 | 0.7 | 98.9 | 4.0 | 94.7 | 35.5 | 58.4 | 6.1 | 2.3 | 0.9 | 0.2 | NT | 781 |
| | 6 | 0.8 | 98.6 | 4.8 | 94.0 | 44.7 | 48.6 | 6.8 | 2.2 | 0.9 | 0.2 | 0.84 | 619 |
| F2 | 0 | 0.7 | 99.3 | 2.3 | 96.8 | 27.2 | 67.8 | 5.0 | 2.3 | 1.0 | 0.1 | 1.04 | 1007 |
| | 0.5 | 0.6 | 99.3 | 2.7 | 96.1 | 28.4 | 65.7 | 5.9 | 2.3 | 1.1 | 0.1 | NT | 811 |
| | 1 | 0.7 | 99.2 | 2.9 | 95.5 | 28.8 | 65.4 | 5.8 | 2.2 | 1.0 | 0.2 | NT | 667 |
| | 3 | 0.7 | 98.9 | 3.5 | 95.4 | 34.5 | 59.0 | 6.5 | 2.4 | 1.0 | 0.2 | NT | 367 |
| | 6 | 0.8 | 98.5 | 5.0 | 93.9 | 42.1 | 50.2 | 7.7 | 3.0 | 1.1 | 0.2 | 0.92 | 0 |

Note:
Values are rounded; therefore, the totals may not equal 100%.

TABLE 7
Stability Data for Ocrelizumab SC DP Robustness Formulations (F3 and F4) Stored at 25° C./60% RH

| | | | | | | | | | | Subvisible Particles | | | |
|---|---|---|---|---|---|---|---|---|---|---|---|---|---|
| | | | | | | | | | | (Cumulative | | | |
| | | | | | Osmolality | L- | Polysorbate | Protein | | Counts/Container) | | | |
| | Time | | Clarity/ | | (mOsmol/ | Met | 20 | Content | Visible | ≥2 | ≥5 | ≥10 | ≥25 |
| Formulation | (months) | Color | Opalescence | pH | kg) | (mM) | (mg/mL) | (mg/mL) | Particulate | μm | μm | μm | μm |
| F3 | 0 | ≤B7 | ≤Ref I | 5.5 | 401 | 19.4 | 0.86 | 36.4 | PFVP | 1718 | 368 | 31 | 0 |
| | 0.5 | NT | NT | NT | NA | NA | 0.86 | NT | PFVP | 1396 | 307 | 46 | 0 |
| | 1 | NT | NT | NT | NA | NA | 0.81 | NT | PFVP | 2883 | 721 | 115 | 0 |
| | 3 | NT | NT | NT | NA | NA | 0.84 | NT | PFVP | 5712 | 1679 | 322 | 0 |
| | 6 | ≤B7 | ≤Ref I | 5.6 | NA | NA | 0.84 | 36.3 | PFVP | 3849 | 652 | 54 | 8 |
| F4 | 0 | ≤B7 | ≤Ref I | 5.3 | 334 | 12.4 | 0.67 | 39.9 | PFVP | 859 | 184 | 39 | 0 |
| (Target) | 0.5 | NT | NT | NT | NA | NA | 0.65 | NT | PFVP | 2208 | 407 | 69 | 0 |
| | 1 | NT | NT | NT | NA | NA | 0.62 | NT | PFVP | 1304 | 292 | 39 | 0 |
| | 3 | NT | NT | NT | NA | NA | 0.65 | NT | PFVP | 4953 | 1150 | 154 | 0 |
| | 6 | ≤B7 | ≤Ref I | 5.4 | NA | NA | 0.65 | 39.9 | PFVP | 2737 | 437 | 62 | 0 |

| | | | | Non-Reduced | | | | | | | | | |
|---|---|---|---|---|---|---|---|---|---|---|---|---|---|
| | | SE-UHPLC | | CE-SDS | | | | | | | | | |
| | | Sum | | Sum | | | | | | | | | |
| | | of | | of | | | | | | | | Potency | |
| | | HMW | | LMW | | IE-HPLC | | | Peptide Map | | | by | |
| | | For | Main | For | Main | Acidic | Main | Basic | HC- | HC- | HC- | CDC | |
| | | ms | Peak | ms | Peak | Region | Peak | Region | M257 | M433 | W107 | Bioassay | rHuPH20 |
| | Time | (area | (area | (% | (% | (area | (area | (area | Oxidation | Oxidation | Oxidation | (e4 U/ | Activity |
| Formulation | (months) | %) | %) | CPA) | CPA) | %) | %) | %) | (%) | (%) | (%) | mg) | (U/mL) |
| F3 | 0 | 0.7 | 99.2 | 2.3 | 96.8 | 27.4 | 67.6 | 5.1 | 2.3 | 1.1 | 0.2 | 1.03 | 1029 |
| | 0.5 | 0.7 | 99.2 | 2.6 | 96.2 | 29.3 | 65.3 | 5.4 | 2.2 | 1.2 | 0.2 | NT | 1029 |
| | 1 | 0.7 | 99.2 | 2.8 | 95.5 | 29.4 | 65.3 | 5.4 | 2.1 | 1.1 | 0.2 | NT | 1071 |
| | 3 | 0.7 | 98.9 | 4.0 | 94.7 | 37.3 | 56.9 | 5.8 | 2.2 | 1.0 | 0.2 | NT | 866 |
| | 6 | 0.9 | 98.6 | 4.6 | 94.3 | 47.3 | 46.7 | 6.0 | 3.0 | 1.2 | 0.2 | 0.82 | 718 |
| F4 | 0 | 0.7 | 99.3 | 2.3 | 96.7 | 27.5 | 67.4 | 5.1 | 2.1 | 0.9 | 0.1 | 1.08 | 1019 |
| (Target) | 0.5 | 0.7 | 99.2 | 2.6 | 96.2 | 27.8 | 66.4 | 5.8 | 2.1 | 1.0 | 0.2 | NT | 982 |
| | 1 | 0.7 | 99.2 | 2.8 | 95.3 | 29.1 | 65.1 | 5.8 | 2.0 | 0.9 | 0.2 | NT | 1002 |
| | 3 | 0.7 | 99.0 | 4.1 | 94.6 | 35.6 | 58.5 | 5.9 | 2.2 | 0.9 | 0.2 | NT | 789 |
| | 6 | 0.8 | 98.6 | 4.8 | 94.1 | 44.5 | 48.6 | 6.9 | 2.7 | 1.0 | 0.2 | 0.84 | 595 |

Note:
Values are rounded; therefore, the totals may not equal 100%.

TABLE 8
Degradation Rate for Ocrelizumab SC DP Robustness Formulations at 25° C.

| | | | Non-Reduced | | | | | | |
|---|---|---|---|---|---|---|---|---|---|
| | SE-UHPLC | | CE-SDS | | | | | Polysorbate | rHuPH20 |
| | (area % | | (% CPA | | | | | 20 | Activity |
| | change/day) | | change/day) | | IE-HPLC | | | (mg/mL | (U/mL |
| | Sum of | | Sum of | | (area % change/day) | | | change | change |
| | HMW | Main | LMW | Main | Acidic | Main | Basic | per | per |
| Formulation | Forms | Peak | Forms | Peak | Region | Peak | Region | day)^{a} | day) |
| F1 | 0.001 | −0.004 | 0.014 | −0.015 | 0.097 | −0.105 | 0.007 | 0.000 | −2.5 |
| (Target) | | | | | | | | | |
| F2 | 0.001 | −0.004 | 0.014 | −0.014 | 0.082 | −0.095 | 0.013 | 0.000 | −5.2 |
| F3 | 0.001 | −0.003 | 0.013 | −0.012 | 0.110 | −0.115 | 0.005 | 0.000 | −1.9 |
| F4 | 0.001 | −0.004 | 0.014 | −0.013 | 0.097 | −0.104 | 0.008 | 0.000 | −2.4 |
| (Target) | | | | | | | | | |

^{a}Little or no changes were observed at 25° C./60% RH.

TABLE 9
Summary of Observed Changes in Ocrelizumab SC
Drug Product after 6 Months at 25° C./60% RH

| Formulation F1 & F4 | | |
|---|---|---|
| (Target) | Formulation F2 | Formulation F3 |
| Changes were observed for: | Changes were observed for: | Changes were observed for: |
| IE-HPLC: | IE-HPLC: | IE-HPLC: |
| Up to 17.5% increase in | 14.9% increase in acidic | 19.9% increase in acidic |
| acidic region | region | region |
| Up to 19.0% decrease in | 17.6% decrease in main | 20.9% decrease in main |
| main peak | peak | peak |
| Up to 1.8% increase in | 2.7% increase in basic | 0.9% increase in basic |
| basic region | region | region |
| SE-UHPLC | SE-UHPLC: | SE-UHPLC: |
| Up to 0.1% increase in the | 0.1% increase in the sum | 0.2% increase in the sum |
| sum of HMW forms | of HMW forms | of HMW forms |
| Up to 0.7% decrease in | 0.8% decrease in main | 0.6% decrease in main |
| main peak | peak | peak |
| NR CE-SDS | NR CE-SDS | NR CE-SDS |
| Up to 2.5% increase in the | 2.7% increase in sum of | 2.3% increase in the sum |
| sum of LMW forms | LMW forms | of LMW forms |
| Up to 2.9% decrease in | 2.9% decrease in main | 2.5% decrease in main |
| main peak | peak | peak |
| Peptide Map | Peptide Map | Peptide Map |
| Up to 0.6% increase in the | Up to 0.7% increase in the | 0.7% increase in the HC- |
| HC-M257 | HC-M257 | M257 |
| Up to 0.1% increase in the | Up to 0.1% increase in the | 0.1% increase in the HC- |
| HC-M433 | HC-M433 | M433 |
| Up to 0.1% increase in the | Up to 0.1% increase in the | 0.0% increase in the HC- |
| HC-W107 | HC-W107 | W107 |
| rHuPH20 Activity | rHuPH20 Activity | rHuPH20 Activity: |
| Up to 452 U/mL decrease | No activity detected after | 311 U/mL decrease in |
| in rHuPH20 activity | 6 months | rHuPH20 activity |
| Potency by CDC Bioassay | Potency by CDC Bioassay | Potency by CDC Bioassay |
| Up to 0.24 e4 U/mg | 0.12 e4 U/mg decrease in | 0.21 e4 U/mg decrease in |
| decrease in potency | potency | potency |

Note:
Little or no changes were observed for all other attributes tested.

### Conclusion

Data obtained from this study for up to six months were analyzed and summarized in this report. The study demonstrates that the commercial ocrelizumab SC drug product formulation is stable across the formulation ranges. Although changes were observed at 25° C., the degradation observed between the target and F2 and F3 formulations was similar (i.e., results in product quality that is acceptable and meet the end of shelf life specification). Variation across the formulation robustness ranges results in similar product quality and meet the end-of-shelf life specifications, including allowable excursions; thereby demonstrating that the formulation is robust.

Part B. a Comparability Assessment of Ocrelizumab SC 920 mg/23 mL Drug Product (DP) and Ocrelizumab IV 300 mg/10 mL Drug Product

### Materials and Methods

The Phase III formulation for ocrelizumab SC formulation is 40 mg/mL ocrelizumab in 20 mM sodium acetate, 240 mM trehalose, 0.6 mg/mL PS20, 10 mM L-Methionine, pH 5.3 with 1000 U/mL rHuPH20. The formulation for ocrelizumab IV DP is 30 mg/mL in 20 mM sodium acetate, 106 mM trehalose, 0.2 mg/mL PS20, pH 5.3. The two formulations were put on stability side by side at 40° C./75% RH for stressed comparability assessment in this study.

The samples were assayed for product quality by color, clarity/opalescence, pH, protein content, visible particles, subvisible particles, size-exclusion high-performance liquid chromatography (SE-UPLC), ion-exchange high-performance liquid chromatography (IE-HPLC), non-reduced capillary electrophoresis sodium dodecyl sulfate (TAMRA NR CE-SDS), polysorbate 20 content, and potency.

### Results

The stability results after storage for 28 days at 40° C./75% relative humidity (Table 10) demonstrate the following:

Little or no changes were observed in the two different vial configurations with respect to pH, color/opalescence/clarity (COC), protein content, visible particles, polysorbate 20 content, and subvisible particles after storage at 40° C./75% relative humidity for 28 days.

Molecular size distribution, as measured by SE-UHPLC, changed with increased exposure to 40° C.: In the main peak, a decrease of 0.6% for the SC formulation and a decrease of 0.6% for the IV formulation was observed between the T0 and T28D time points. For the HMWS, no change for the SC formulation and an increase of 0.1% for the IV formulation was observed between the TO and T28D time points. For the LMWS, an increase of 0.4% for the SC formulation and an increase of 0.4% for the IV formulation were observed between the T0 and T28D time points. A similar rate of change in the main peak determined by linear regression was observed with −0.02% per day for the SC formulation and −0.02% per day for the IV formulation. A rate of change in the HMWS peak determined by linear regression was observed with 0.003% per day for the SC formulation and 0.006% per day for the IV formulation. Slightly better stability is observed for the SC formulation. The chromatographic profiles between the two formulations show similar peak shapes and rank order; no new peaks were observed. (Data not shown.)

The molecular size distribution, as measured by non-reduced CE-SDS, changed with increased exposure to 40° C.: In the main peak, a decrease of 2.1% for the SC formulation and a decrease of 2.7% for the IV formulation was observed between the TO and T28D time points. For the sum of the pre-peaks, an increase of 2.2% for the SC formulation and an increase of 2.5% for the IV formulation was observed between the TO and T28D time points. A rate of change in the main peak determined by linear regression was observed with −0.1% per day for the SC formulation and −0.1% per day for the IV formulation. A rate of change in the sum of the pre-peak determined by linear regression was observed with 0.1% per day for the SC formulation and 0.1% per day for the IV formulation. The electrophoretic profiles between the two formulations show similar peak shapes and rank order; no new peaks were observed.

The distribution of charge variants, as measured by IE-HPLC, changed with increased exposure to 40° C.: In the main peak, a decrease of 22.9% for the SC formulation and a decrease of 23.5% for the IV formulation was observed between the TO and T28D time points. In the acidic region, an increase of 22.2% for the SC formulation and an increase of 22.2% for the IV formulation was observed between the TO and T28D time points. A rate of change in the main peak determined by linear regression was observed with −0.8% per day for the SC formulation and −0.8% per day for the IV formulation. A rate of change in the acidic region determined by linear regression was observed with 0.8% per day for the SC formulation and 0.8% per day for the IV formulation. The chromatographic profiles between the two formulations show similar peak shapes and rank order; no new peaks were observed.

A decrease is potency of 0.19×10⁴ U/mg was observed for the SC formulation and a decrease of 0.21×10⁴ U/mg was observed for the IV formulation from the T0 to T28D time points.

The SC formulation appears to show slightly better stability in size variants (e.g., HMWS by SE-UPLC and LMWS by NR CE-SDS). Overall, the degradation rates between the ocrelizumab SC DP and ocrelizumab IV DP vials are highly similar.

TABLE 10
Stability Data for Ocrelizumab IV vs SC formulation at 40° C.

| | | | | | | | Sub-visible | | | | Sub-visible | | | | PS20 |
|---|---|---|---|---|---|---|---|---|---|---|---|---|---|---|---|
| | | | | | | | Particulates | | | | Particulates(particles | | | | Content |
| | | COC | | | | Protein | (particles per mL) | | | | per container) | | | | by |
| Batch | Time | | Clarity/ | Visible | | Concentration | ≥2 | ≥5 | ≥10 | ≥25 | ≥2 | ≥5 | ≥10 | ≥25 | ELSD |
| Number | (months) | Color | Opalescence | Particulate | pH | (mg/mL) | μm | μm | μm | μm | μm | μm | μm | μm | (mg/mL) |
| IV | 0 | ≤B6 | ≤Ref I | PFFP | 5.3 | 30.7 | 63 | 21 | 5 | 0 | 627 | 207 | 50 | 0 | 0.21 |
| | 7 | ≤B6 | ≤Ref I | PFFP | NT | NT | 106 | 30 | 4 | 0 | 1060 | 300 | 34 | 0 | 0.21 |
| | 14 | ≤B6 | ≤Ref I | PFFP | NT | NT | 171 | 45 | 7 | 0 | 1710 | 447 | 64 | 0 | 0.21 |
| | 28 | ≤B6 | ≤Ref I | PFFP | 5.3 | 30.7 | 51 | 7 | 2 | 0 | 510 | 67 | 14 | 0 | 0.21 |
| SC | 0 | ≤B6 | ≤Ref I | PFFP | 5.3 | 39.8 | 151 | 32 | 3 | 0 | 3473 | 729 | 62 | 0 | 0.57 |
| | 7 | ≤B6 | ≤Ref I | PFFP | NT | NT | 235 | 52 | 6 | 0 | 5398 | 1181 | 138 | 0 | 0.61 |
| | 14 | ≤B6 | ≤Ref I | PFFP | NT | NT | 154 | 32 | 4 | 0 | 3527 | 736 | 92 | 0 | 0.50 |
| | 28 | ≤B6 | ≤Ref I | PFFP | 5.3 | 39.9 | 165 | 29 | 5 | 0 | 3788 | 660 | 100 | 0 | 0.60 |

COC = clarity, opalescence, Color; PS20 = polysorbate 20; ELSD = evaporative light scattering detector; NT = not tested; PFFP = practically free from particles; The nominal fill volume for IV DP is 10 mL and for SC DP is 23 mL.

### Conclusion

The data provided demonstrate that Ocrelizumab SC and Ocrelizumab IV have comparable stability at the stressed conditions of 40° C./75% relative humidity. Therefore, shelf life between Ocrelizumab SC and Ocrelizumab IV should be comparable to each other.

### Part C. Stability of Drug Product Ocrelizumab SC for Long-Term Conditions.

This study provides representative stability data of Phase III ocrelizumab SC Drug Product. Tables 11-12 contain data from ocrelizumab SC Drug Product Lot X. The Drug Product is composed of 40 mg/mL ocrelizumab and 1000 U/mL rHuPH20 (standard formulation) in 20 mM sodium acetate, 240 mM trehalose, 10 mM L-methionine, 0.06% (w/v) polysorbate 20 at pH 5.3. The Drug Product configuration used in this study is a nominal 10 mL fill in 20 cc glass vials.

Ocrelizumab SC Drug Product was analyzed by color, opalescence, and clarity, pH, strength, osmolality, visible particles, enhanced visual inspection, subvisible particles, PS20 content, SE-HPLC/SE-UPLC, IE-HPLC, non-reduced CE-SDS (using TAMRA dye), reduced CE-SDS (using TAMRA dye), potency (CDC and ADCC activity), oxidation, methionine content, and rHuPH20 activity.

Conclusion: The ocrelizumab SC drug product has acceptable stability over 36 months at 2-8° C. Therefore, the drug product shelf-life is set at 36 months for clinical use.

TABLE 11
Stability Data for Drug Product Ocrelizumab SC
Technical Batch X at Long-Term Conditions 5° C.

| | | | | Subvisible | | | | SE-UHPLC | |
|---|---|---|---|---|---|---|---|---|---|
| | | | | Particles (per | | | Content of | (area %) | |
| | | | | container) | | | Polysorbate | | Sum of |
| Time | | Clarity/ | Visible | ≥10 | ≥25 | | 20 | Main | HMW |
| (months) | Color | Opalescence | Particles | μm | μm | pH | (mg/mL) | Peak | Forms |
| 0 | ≤B7 | ≤Ref I | PFFP | 127 | 4 | 5.3 | 0.6 | 99.4 | 0.5 |
| 3 | — | — | PFFP | 30 | 7 | — | 0.6 | 99.3 | 0.5 |
| 6 | ≤B7 | ≤Ref I | PFFP | 24 | 0 | 5.3 | 0.5 | 99.3 | 0.5 |
| 9 | — | — | PFFP | 20 | 0 | — | 0.5 | 99.3 | 0.5 |
| 12 | ≤B8 | ≤Ref I | PFFP | 290 | 64 | 5.3 | 0.5 | 99.3 | 0.5 |
| 18 | ≤B6 | ≤Ref I | PFFP | 7 | 0 | 5.3 | 0.5 | 99.2 | 0.6 |
| 24 (I) | ≤B6 | ≤Ref I | PFFP | 77 | 7 | 5.3 | 0.6 | 99.2 | 0.6 |
| 24 (U) | ≤B6 | ≤Ref I | — | 20 | 7 | 5.3 | — | 99.2 | 0.6 |
| 30 | ≤B7 | ≤Ref I | PFFP | 64 | 0 | 5.3 | 0.6 | 99.1 | 0.6 |
| 36 (I) | ≤B6 | ≤Ref I | PFFP | 4 | 0 | 5.3 | 0.6 | 99.1 | 0.6 |
| 36 (U) | ≤B6 | ≤Ref I | — | 7 | 4 | 5.3 | 0.5 | 99.1 | 0.6 |

This study provides representative stability data of Phase III ocrelizumab SC Drug Product. Tables 11-12 contain data from ocrelizumab SC Drug Product Lot X. The Drug Product is composed of 40 mg/mL ocrelizumab and 1000 U/mL rHuPH20 (standard formulation) in 20 mM sodium acetate, 240 mM trehalose, 10 mM L-methionine, 0.06% (w/v) polysorbate 20 at pH 5.3. The Drug Product configuration used in this study is a nominal 10 mL fill in 20 cc glass vials.

Ocrelizumab SC Drug Product was analyzed by color, opalescence, and clarity, pH, strength, osmolality, visible particles, enhanced visual inspection, subvisible particles, PS20 content, SE-HPLC/SE-UPLC, IE-HPLC, non-reduced CE-SDS (using TAMRA dye), reduced CE-SDS (using TAMRA dye), potency (CDC and ADCC activity), oxidation, methionine content, and rHuPH20 activity.

Conclusion: The ocrelizumab SC drug product has acceptable stability over 36 months at 2-8° C. Therefore, the drug product shelf-life is set at 36 months for clinical use.

TABLE 11
Stability Data for Drug Product Ocrelizumab SC Technical Batch X at
Long-Term Conditions 5° C. (cont.)

| | Non-Reduced | | | | | | | |
|---|---|---|---|---|---|---|---|---|
| | CE-SDS | | | | | | | |
| | (%CPA) | | | | | | | |
| Time | | Sum of | IE-HPLC (area %) | | | Protein | | Activity of |
| (mont | Main | LMW | Main | Acidic | Basic | Content | Potency | rHuPH20 |
| hs) | Peak | Forms | Peak | Region | Region | (mg/mL) | (e4 U/mg) | (U/mL) |
| 0 | 96.4 | 2.8 | 64.3 | 29.4 | 6.3 | 40.6 | 1.11 | 1165 |
| 3 | 95.9 | 3.4 | 64.2 | 28.8 | 7.0 | 40.6 | 1.16 | 1105 |
| 6 | 96.6 | 2.5 | 64.1 | 28.2 | 7.8 | 40.0 | 0.92 | 1136 |
| 9 | 96.4 | 2.7 | 65.2 | 28.8 | 6.0 | — | — | 1096 |
| 12 | 96.4 | 2.8 | 64.9 | 29.1 | 6.0 | 40.3 | 0.97 | 1119 |
| 18 | 96.2 | 3.0 | 62.0 | 28.8 | 9.2 | 37.2 | 0.96 | 1036 |
| 24 (I) | 96.6 | 2.6 | 62.2 | 30.4 | 7.4 | 40.0 | 1.02 | 936 |
| 24 (U) | 96.5 | 2.7 | 60.8 | 29.6 | 9.5 | 40.7 | 0.93 | 952 |
| 30 | — | — | 62.8 | 31.3 | 5.9 | 39.7 | 0.92 | 990 |
| 36 (I) | 96.2 | 3.0 | 62.7 | 31.0 | 6.3 | 40.7 | 0.93 | 1006 |
| 36 (U) | 96.5 | 2.7 | 62.8 | 31.0 | 6.2 | 40.6 | 0.97 | 1039 |

^{a} SE-UHPLC replaced SE-HPLC method after the stability studies started. The results were obtained by testing the frozen reserved samples that were stored at −60° C. or below.

TABLE 12
Stability Data for Drug Product Technical Batch X at Accelerated Conditions (25° C.)

| Subvisible | | |
|---|---|---|
| Particles | | |
| (per | Content of | SE-UHPLC (area %) |

| | | | | container) | | | Polysorbate | | Sum of |
|---|---|---|---|---|---|---|---|---|---|
| Time | | Clarity/ | Visible | ≥10 | ≥25 | | 20 | Main | HMW |
| (months) | Color | Opalescence | Particles | μm | μm | pH | (mg/mL) | Peak | Forms |
| 0 | ≤B7 | ≤Ref I | PFFP | 127 | 4 | 5.3 | 0.6 | 99.4 | 0.5 |
| 0.5 | — | — | — | — | — | — | 0.6 | 99.3 | 0.5 |
| 1 | — | — | PFFP | 24 | 0 | — | 0.6 | 99.2 | 0.5 |
| 3 | ≤B7 | ≤Ref I | PFFP | 10 | 0 | 5.3 | 0.6 | 99.1 | 0.6 |
| 6 | ≤B7 | ≤Ref I | PFFP | 24 | 0 | 5.3 | 0.5 | 98.8 | 0.6 |

| | Non-Reduced | | | | | | | | |
|---|---|---|---|---|---|---|---|---|---|
| | CE-SDS | | | | | | | | |
| | (% CPA) | | | | | | | | Activity |
| | | Sum of | IE-HPLC (area %) | | | Protein | | | of |
| Time | Main | LMW | Main | Acidic | Basic | Content | Potency | Osmolality | rHuPH20 |
| (months) | Peak | Forms | Peak | Region | Region | (mg/mL) | (e4 U/mg) | mOsmo/kg | (U/mL) |
| 0 | 96.4 | 2.8 | 64.3 | 29.4 | 6.3 | 40.6 | 1.11 | 330 | 1165 |
| 0.5 | 96.1 | 3.1 | 63.0 | 30.3 | 6.7 | — | — | — | 1022 |
| 1 | 96.2 | 3.2 | 61.5 | 31.5 | 7.0 | — | 1.11 | — | 902 |
| 3 | 94.9 | 4.3 | 55.5 | 36.9 | 7.7 | 40.5 | 0.94 | 332 | 543 |
| 6 | 95.0 | 4.2 | 47.6 | 43.7 | 8.7 | 40.6 | 1.02 | 327 | 328 |

### Example 2: A Phase Ib Study to Investigate the Pharmacokinetics, Safety, and Tolerability of Subcutaneous Ocrelizumab Administration in Patients with Multiple Sclerosis

This study evaluated the pharmacokinetics, safety and tolerability, and immunogenicity of ocrelizumab administered subcutaneously to patients with MS. Specific objectives and corresponding endpoints for the study are outlined in the following sections.

The primary pharmacokinetic (PK) objective of this study was to compare the PK profile of SC ocrelizumab with IV ocrelizumab on the basis of the following endpoints: a) Serum concentration of ocrelizumab at specified timepoints following single SC administration, by determining the area under the concentration-time curve (AUC); b) Serum concentration of ocrelizumab at specified timepoints following single IV administration (i.e., AUC).

The safety objective of this study was to evaluate and compare the safety and tolerability profile after administration of SC ocrelizumab with 600 mg of IV ocrelizumab in patients with MS on the basis of the following endpoints during the duration of the study: a) Incidence and severity of adverse events, with severity determined according to the National Cancer Institute Common Terminology Criteria for Adverse Events, Version 5.0 (NCI CTCAE v5.0); b) Change from baseline in targeted vital signs; c) Change from baseline in ECG parameters; d) Incidence and severity of clinical laboratory abnormalities; e) Incidence of local pain at the site of injection and local injection-site reaction (ISR), assessed using the Visual Analog Scale (VAS) and Local Injection-Site Symptom Assessment (LISSA).

The immunogenicity objective for this study was to evaluate the formation of anti-drug antibodies (ADAs) to SC and IV administered ocrelizumab and the formation of ADAs to rHuPH20 on the basis of the following endpoints: a) Incidence of treatment-emergent ADAs during the duration of the study relative to the presence of ADAs at baseline; b) The relationship between ADA status and pharmacokinetics and safety.

### Dose Escalation Phase

Group A (patients pretreated with ocrelizumab) involved non-randomized cohorts, e.g., cohorts A1-A4. Cohort A1: 40 mg of SC ocrelizumab; Cohort A2: 200 mg of SC ocrelizumab; Cohort A3: 600 mg of SC ocrelizumab; Cohort A4: 1200 mg of SC ocrelizumab. In the non-randomized subphase, participants in Cohort A5 received a single SC injection of ocrelizumab co-mixed with rHuPH20 in the abdomen. For Cohort AA, Participants received a single 600-mg dose ocrelizumab by intravenous (IV) infusion.

Patients enrolled in Group B (Ocrelizumab treatment-naive participants) received a single SC injection of ocrelizumab co-mixed with rHuPH20 in the abdomen. Specifically, cohort B1: 40 mg of SC ocrelizumab. Cohort B2: 200 mg of SC ocrelizumab. Cohort B3: 600 mg of SC ocrelizumab. Cohort B4: 1200 mg of SC ocrelizumab.

To reduce the frequency and severity of IRRs, as well as systemic injection reactions or ISRs, patients were premedicated with corticosteroids and antihistamines prior to administration with ocrelizumab.

An emerging safety profile of injection reactions (predominantly local and some systemic) has been characterized by mild to moderate severity and decreasing incidence after the first injection. Should reactions occur, they are manageable according to the current guidelines to be performed in the event of severe systemic injection reactions including potential hypersensitivity/anaphylactic reactions. In the study, the most common systemic injection reaction symptom was headache. The median and mean time to onset of systemic injection reaction symptoms were approximately 5 hours and 6 hours after administration of ocrelizumab SC injection, respectively.

Given these data, and in order to improve patients' experience and limit time on site, it was considered it is no longer necessary to wait 1-2 hours between administering oral premedications and SC ocrelizumab injection. Oral premedications in some cases were administered shortly before initiating the ocrelizumab SC injection.

Safety information from the study shows a decreasing incidence of local and systemic injection reactions after the first ocrelizumab SC injection. All local reactions to date have been CTCAE Grade 1-2 and non-serious. Systemic injection reactions with onset less than 1 hour after the start of injection have been mild or moderate in intensity, non-serious, and resolved with standard treatments. No Grade 3 systemic reactions have been observed from the second injection onwards. Data from the study does not indicate a ‘window’ of greatest risk after SC administration.

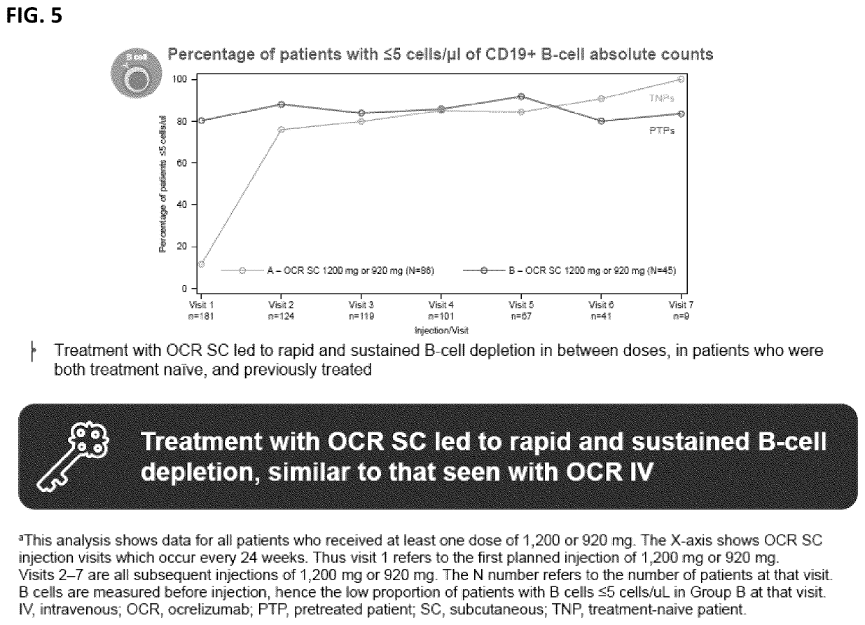

Based upon the PK and safety data obtained in this study, the dose of 920 mg SC was selected for the study described in Example 3. In this study, SC doses up to 1200 mg have been administered and were safe and well tolerated. SC bioavailability was estimated to be 72%. Rapid B cell depletion in ocrelizumab treatment-naïve patients, and maintenance of B cell depletion throughout the six-month dosing interval in pre-treated patients, was obtained after 1200 mg ocrelizumab SC. See e.g., FIG. 5, which shows that treatment with OCR SC led to rapid and sustained B-cell depletion in between doses, in patients who were both treatment naïve, and previously treated.

In cohort A (n=53 OCR SC; n=35 OCR IV) and cohort B OCR SC (n=46), the majority were female (72.7%/63.0%); mean age (standard deviation) at baseline was 45.7 (10.2) and 39.7 (9.2) years, respectively. During the dose escalation phase, OCR SC was well tolerated across all doses tested. Initially 1200 mg was selected as the candidate SC dose, but subsequently 920 mg was chosen as the final SC dose based on all data available. Median treatment duration with OCR SC 1200 or 920 mg was 96 weeks, with 94.7% receiving ≥3 doses. Injection site reactions were the most common adverse events, with erythema, pain, and swelling being the most common symptoms, all of which were mild/moderate.

The SC dose of 920 mg was selected such that the ocrelizumab exposure in patients after SC administration matches the exposure after the approved and marketed dose of 600 mg IV. As variability after SC administration is larger than after IV administration, the SC dose of 920 mg was selected so that the 25th percentile in the exposure (AUCt) distribution matches the 25th percentile in the exposure distribution after IV 600 mg dosing. This ensures that patients on the lower range of the exposure distribution have adequate exposure.

For the IV administration, the initial 600 mg dose is divided into 2×300 mg. For SC administration, this is not required, as one single dose of up to 1200 mg was well tolerated in ocrelizumab treatment-naïve patients. Therefore, all SC doses in the study described in Example 3 were given as a single SC injection. Absorption after SC injection is slow (Cₘₐₓ between Day 2 and Day 10, based on currently available data (data not shown). SC injection of the total dose on Day 1 help to achieve the onset of efficacy with similar speed versus the IV infusions, for which the systemic availability of the total dose is complete on Day 14.

Treatment Administration Satisfaction Questionnaire (TASQ) data demonstrated a high level of satisfaction for OCR SC; most patients who received OCR SC 1,200 mg or 920 mg reported being satisfied with the SC treatment administration. Specifically, the majority of patients were either satisfied or very satisfied with the SC procedure (n=92, 93.9%). The majority of patients felt that the SC procedure was convenient or very convenient (n=86, 87.8%). The majority of patients felt that the time taken to get the injection was just right (n=92, 93.9%).

### Example 3: A Phase III Study to Investigate the Pharmacokinetics, Pharmacodynamics, Safety and Radiological and Clinical Effects of Subcutaneous Ocrelizumab Versus Intravenous Ocrelizumab in Patients with Multiple Sclerosis

### Objectives and Endpoints

This study evaluated the pharmacokinetics, pharmacodynamics, safety, immunogenicity, and radiological and clinical effects of subcutaneous (SC) administration of ocrelizumab compared with the intravenous (IV) infusion of ocrelizumab in patients with either relapsing multiple sclerosis (RMS) or primary progressive multiple sclerosis (PPMS). Specific objectives and corresponding endpoints for the study were as following and outlined below. Specifically, the primary endpoint is non-inferiority in serum area area under the curve (AUC) from day 1 to 12 weeks after subcutaneous injection compared to IV infusion. Secondary endpoints include maximum serum concentration (Cmax) of ocrelizumab, the total number of active, gadolinium-enhancing T1 lesions at 8 and 12 weeks, and new or enlarging T2 lesions at 12 and 24 weeks, as well as safety, immunogenicity and biomarker outcomes.

### Primary Pharmacokinetic Objective

The primary PK objective of this study was to demonstrate PK non-inferiority of the SC formulation of ocrelizumab in patients with multiple sclerosis (MS) on the basis of the following endpoint: serum ocrelizumab area under the concentration-time curve (AUC_{W1-12}) after SC administration compared to IV infusion from Day 1 to Week 12.

### Secondary Pharmacokinetic Objective

The secondary PK objective of this study was to determine the maximum serum concentration (Cmax) of ocrelizumab SC in patients with MS.

### Secondary Radiological Objective

The secondary radiological objective for this study is to evaluate the radiological effects of ocrelizumab SC compared with ocrelizumab IV in patients with MS on the basis of the following endpoints:
1. Total number of T1Gd+ lesions as detected by brain MRI at Weeks 8 and 24.
2. Total number of new or enlarging T2 lesions as detected by brain MRI at Weeks 12 and 24 relative to the previous scan, respectively.

### Safety Objective

To evaluate and compare the safety profile after administration of OCR SC versus OCR IV and to assess the safety of OCR SC at the selected dose in patients with MS based on the following endpoints during the conduct of the study:
1. Incidence and severity of adverse events, with severity determined according to the NCI CTCAE, version 5.0.
2. Change from baseline in targeted vital signs.
3. Change from baseline in targeted clinical laboratory test results.

### Immunogenicity Objective

To evaluate the immune response to OCR SC and IV, and rHuPH20, based on the following endpoints:
1. Incidence and/or formation of treatment-emergent ADAs to OCR SC vs OCR IV relative to the presence of ADAs at baseline.
2. Relationship between ADA status to OCR and PK, PD, and safety.
3. Incidence of treatment-emergent antibodies to rHuPH20 after SC administration relative to the presence at baseline, and relationships between antibodies to rHuPH20 and safety.

### Pharmacodynamic Objective

To evaluate the effect of OCR SC compared with OCR IV on the PD marker for the mechanism of action of OCR (i.e., B-cell depletion) based on the endpoint of the proportion of patients achieving CD19+ B-cell level≤5 cells/μL at Weeks 12, 24, 48, and/or 96. Biomarker Objective

To evaluate biomarkers that are predictive of response to OCR (i.e., predictive biomarkers), are early surrogates of efficacy, are associated with progression to a more severe disease state (i.e., prognostic biomarkers), can provide evidence of OCR activity (i.e., PD markers), or can increase the knowledge and understanding of disease biology, based on the endpoint of the levels of biomarkers including but not limited to NfL in serum compared between dosing arms at Weeks 12, 24, 48, and/or 96 compared to baseline.

### Patient Population and Criteria

Number of Patients: Approximately 232 patients were enrolled with different types of MS (RMS and PPMS).

### Inclusion Criteria:

Diagnosis of PPMS or RMS according to the revised McDonald 2017 criteria (Thompson et al. 2018).

### Signed Informed Consent Form.

Age 18-65 years, inclusive, at time of signing Informed Consent Form.

Ability to comply with the study protocol and schedule of protocol assessments, in the investigator's judgment.

EDSS score, 0-6.5, inclusive, at screening.

Neurological stability for ≥30 days prior to both screening and baseline.

Disease duration from onset of MS symptoms of less than 15 years for patients with EDSS score <2.0 at screening.

For women of childbearing potential: agreement to remain abstinent (refrain from heterosexual intercourse) or use adequate contraception during the treatment period and for 6 to 12 months (as applicable by the OCR IV [Ocrevus®] local label) after the final dose of OCR. Adherence to local requirements, if more stringent, is required.

A woman is considered to be of childbearing potential if she is postmenarchal, has not reached a post-menopausal state (≥12 continuous months of amenorrhea with no identified cause other than menopause), and is not permanently infertile due to surgery (i.e., removal of ovaries, fallopian tubes, and/or uterus) or another cause as determined by the investigator (e.g., Müllerian agenesis). Per this definition, a woman with a tubal ligation is considered to be of childbearing potential. The definition of childbearing potential may be adapted for alignment with local guidelines or requirements.

As defined by the guidelines, the following contraceptive methods are considered acceptable (failure rate >1%): (1) progesterone-only hormonal contraception, where inhibition of ovulation is not the primary mode of action; (2) male or female condom with or without spermicide; (3) cap, diaphragm, or sponge with spermicide. (4) combination of male condom with cap, diaphragm, or sponge with spermicide (double-barrier method).

Birth control methods that are highly effective (failure rate <1%) may also be used but are not required, and include: (1) oral, intravaginal or transdermal combined hormonal contraception associated with inhibition of ovulation; (2) oral, injectable or implantable progestogen-only hormonal contraception associated with inhibition of ovulation; (3) intrauterine device; (4) intrauterine hormone-releasing system; (5) bilateral tubal occlusion; (6 vasectomized partner; (7) sexual abstinence.

The reliability of sexual abstinence should be evaluated in relation to the duration of the clinical trial and the preferred and usual lifestyle of the patient. Periodic abstinence (e.g., calendar, ovulation, symptothermal, or postovulation methods) and withdrawal are not acceptable methods of contraception. If required per local guidelines or regulations, locally recognized adequate methods of contraception and information about the reliability of abstinence are described in the local Informed Consent Form.

For female patients without reproductive potential: women may be enrolled if either post-menopausal (i.e., spontaneous amenorrhea for the past year confirmed by a follicle-stimulation hormone level >40 mIU/mL) unless the patient is receiving a hormonal therapy for her menopause, or surgically sterile (i.e., hysterectomy, complete bilateral oophorectomy).

### Exclusion Criteria:

Any known or suspected active infection at screening or baseline (except nailbed infections), or any major episode of infection requiring hospitalization or treatment with IV antimicrobials within 8 weeks prior to and during screening or treatment with oral antimicrobials within 2 weeks prior to and during screening.

History of confirmed or suspected PML.

History of cancer, including hematologic malignancy and solid tumors, within 10 years of screening.

Basal or squamous cell carcinoma of the skin that has been excised and is considered cured and in situ carcinoma of the cervix treated with apparent success by curative therapy >1 year prior to screening is not exclusionary.

Immunocompromised state, defined as one or more of the following: CD4 count <250/μL. Absolute neutrophil count <1.5×10³/μL. Serum IgG <4.6 g/L.

Receipt of a live-attenuated vaccine within 6 weeks prior to randomization.

Influenza vaccination is permitted if the inactivated vaccine formulation is administered.

Inability to complete an MRI (contraindications for MRI for example, due to, pacemaker, cochlear implants, intracranial vascular clips, surgery within 6 weeks of entry in the study, coronary stent implanted within 8 weeks prior to the time of the intended MRI, etc.) or contraindication to gadolinium administration.

Contraindications to mandatory premedications (i.e., corticosteroids and antihistamines), including closed-angle glaucoma for antihistamines.

Known presence of other neurologic disorders that could interfere with the diagnosis of MS or assessments of efficacy and/or safety during the study, including, but not limited to, the following: 1. History of hemorrhagic or ischemic cerebral or spinal stroke. 2. History or known presence of CNS or spinal cord tumor (e.g., meningioma, glioma). 3. History or known presence of potential metabolic causes of myelopathy (e.g., untreated vitamin B12 deficiency). 4. History or known presence of infectious causes of myelopathy (e.g., syphilis, Lyme disease, HTLV-1, herpes zoster myelopathy). 5. History of genetically inherited progressive CNS degenerative disorder (e.g., hereditary paraparesis, mitochondrial myopathy, encephalopathy, lactic acidosis, stroke [MELAS] syndrome). 6. Neuromyelitis optica. 7. History or known presence of systemic autoimmune disorders potentially causing progressive neurologic disease (e.g., lupus, anti-phospholipid antibody syndrome, Sjögren syndrome, Behçet disease). 8. History or known presence of sarcoidosis. 9. History of severe, clinically significant brain or spinal cord trauma (e.g., cerebral contusion, spinal cord compression).

Any concomitant disease that may require chronic treatment with systemic corticosteroids (e.g. mineralocorticoids and glucocorticoids) or immunosuppressants during the course of the study.

Significant, uncontrolled disease, such as cardiovascular (including cardiac arrhythmia), pulmonary (including obstructive pulmonary disease), renal, hepatic, endocrine or gastrointestinal, or any other significant disease that may preclude patient from participating in the study.

History of or currently active primary or secondary (non-drug-related) immunodeficiency.

Pregnant or breastfeeding, or intending to become pregnant during the study and 6 or 12 months (as applicable by the OCR [Ocrevus®] local label) after last administration of the study drug. Females of childbearing potential must have a negative serum and urine pregnancy test result prior to initiation of study drug (negative serum β-CG measured at screening and negative urine β-CG at baseline).

Lack of peripheral venous access.

History of alcohol or other drug abuse within 12 months prior to screening.

Treatment with any investigational agent within 24 weeks prior to screening or 5 half-lives of the investigational drug (whichever is longer), or treatment with any experimental procedure for MS (e.g., treatment for chronic cerebrospinal venous insufficiency).

Patients who have previously received anti-CD20s (including OCR) if the last treatment was less than 2 years before screening, and/or if B-cell count is below lower limit of normal, and/or the discontinuation of the treatment was due to safety reasons or lack of efficacy.

Previous treatment with cladribine, atacicept, and alemtuzumab.

Previous treatment with fingolimod, siponimod, ponesimod, or ozanimod within 6 weeks of baseline.

Previous treatment with interferons beta (1a or 1b), or glatiramer acetate within 2 weeks of baseline.

Previous treatment with natalizumab within 4.5 months of baseline.

Treatment with mitoxantrone within 2 years prior to baseline visit or evidence of cardiotoxicity following mitoxantrone use or a cumulative lifetime dose of more than 60 mg/m². Previous treatment with any other immunomodulatory or immunosuppressive medication not already listed above without appropriate washout as described in the applicable local label. If the washout requirements are not described in the applicable local label (washout to be completed prior to baseline), then the wash out period must be 5 times the half-life of the medication. The PD effects of the previous medication must also be considered when determining the required time for washout. Patients screened for this study should not be withdrawn from therapies for the sole purpose of meeting eligibility for the trial.

Any previous treatment with bone marrow transplantation and hematopoietic stem cell transplantation.

Any previous history of transplantation or anti-rejection therapy.

Treatment with IV Ig or plasmapheresis within 12 weeks prior to randomization.

Systemic corticosteroid therapy within 4 weeks prior to screening. The screening period may be extended for patients who have used systemic corticosteroids for MS before screening. For a patient to be eligible, systemic corticosteroids should also not have been administered between screening and baseline.

Positive screening tests for active, latent, or inadequately treated hepatitis B (as evidenced by either of the following): 1. Positive hepatitis B surface antigen. 2. Positive hepatitis B core antibody (total HBcAb) and detectable hepatitis B virus DNA.

Sensitivity or intolerance to any ingredient (including excipients) of OCR.

Any additional exclusionary criterion as per OCR (Ocrevus®) label, if more stringent than the above.

### Re-Testing Before Baseline

In rare cases in which the screening laboratory samples are rejected by the central laboratory (e.g., hemolyzed sample) or the result is not assessable (e.g., indeterminate) or abnormal, the tests need to be repeated as soon as possible, or within the allowed screening period. Any abnormal screening laboratory value that is clinically relevant should be retested to rule out any progressive or uncontrolled underlying condition. The last value before randomization must meet study criteria.

### Study Design

Investigational Medicinal Products: OCR comprising both the IV formulation and the SC formulation co-formulated with rHuPH20.
Test Product (Investigational Drug): OCR SC provided as a sterile, single-dose liquid formulation at a concentration of 40 mg/mL containing no preservatives. The formulation described in Example 1 was used for OCR SC in this example.
Comparator: OCR IV provided as a sterile, single-dose liquid at a concentration of 30 mg/mL containing no preservatives. The liquid solution also contains glacial acetic acid (0.25 mg/mL), polysorbate 20 (0.2 mg/mL), sodium acetate trihydrate (2.14 mg/mL), and trehalose dihydrate (40 mg/mL) at pH 5.3.

### Non-Investigational Medicinal Products

Specific premedications are required prior to the ocrelizumab infusion or injection and include mandatory methylprednisolone or dexamethasone, mandatory diphenhydramine or desloratadine, and optional oral analgesics as needed.

### Statistical Methods

### Primary Analysis

The primary objective for this study is to establish the non-inferiority of ocrelizumab SC compared with ocrelizumab IV on the basis of serum ocrelizumab area under the concentration-time curve (AUC) up to Week 12 (AUC_{W1-12}). The geometric mean ratio (GMR) of AUC SC versus AUC IV are presented, together with the two-sided 90% confidence interval. The non-inferiority is established if the lower end of the two-sided 90% CI of the GMR of AUC is >0.8. The non-inferiority limit of 0.8 corresponds to a maximal 20% loss in AUC for the SC administration compared with IV. All available results for PK samples obtained during the first 12 weeks are included in the PK analysis. The primary comparison between SC and IV administered ocrelizumab are based on the AUC_{W1-12} (i.e., over the first 3 months of the 6 monthly dosing interval after first dose), as absorption is expected to be complete by Week 12.

### Treatment Phase

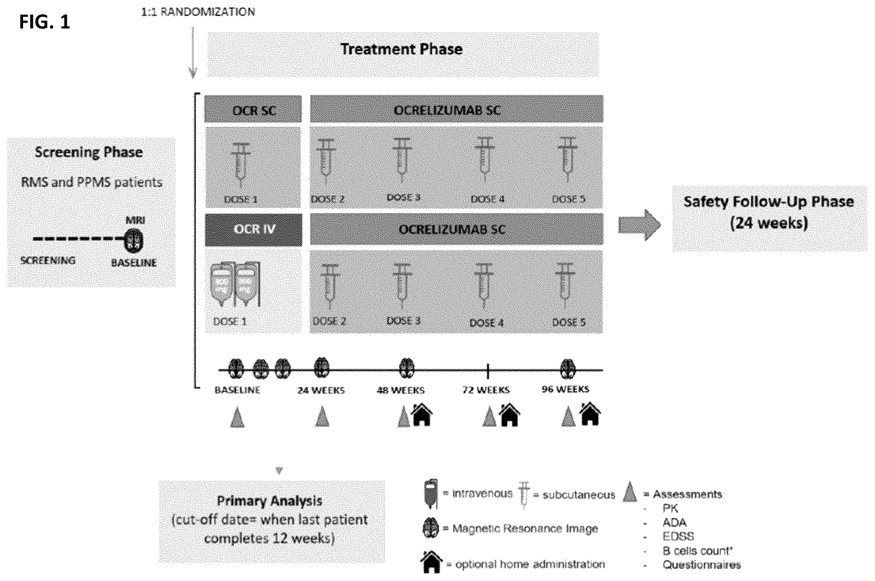

Patients were recruited globally. All patients received five OCR doses (Day 1, Week 24, Week 48, Week 72 and Week 96) during the treatment phase. See e.g., FIG. 1. Eligible patients were randomly assigned to one of two treatment arms: OCR SC or OCR IV. Randomization occurred in a 1:1 ratio and was performed through an interactive voice or web-based response system (IxRS).

The first study drug administration should occur within 24 hours of randomization (in exceptional cases within 48 hours of randomization provided that the investigator assures that all inclusion and exclusion criteria are still met on the day of dosing).

During the controlled period (i.e., time until the Week 24 dose of OCR SC), the first dose of OCR SC was given as one SC injection at a dose of 920 mg. The first dose of OCR IV was administered as two 300 mg IV infusions given 2 weeks apart. Every effort was made to perform all assessments required during the controlled period (e.g., MRI) before the administration of the second dose. The subsequent doses of study drug were administered as SC injections for all patients, including those initially randomized to OCR IV (OCR SC treatment period).

For patients randomized in the OCR IV group, there was a minimum of 20 weeks between the administration of the final OCR IV infusion and the first OCR SC dose (i.e., between the IV infusion at Week 2/Study Day 14 and the first SC dose during Week 24). There was a minimum of 22 weeks between all SC doses.

To allow for the possibility of home administration at participating sites, from the third OCR dose onwards (i.e., Week 48 onwards) the OCR dose could be administered by a healthcare professional at the patient's home or another suitable location.

Two-hundred and thirty-six patients across 37 sites, were randomized to OCR SC (n=118) and IV (n=118). At baseline (BL), mean (standard deviation [SD]) age was 39.9 (11.4)/40.0 (11.9) years, median weight was 75 kg/72 kg and 65.3%/59.3% of patients were female in the SC and IV cohorts, respectively. The majority of participants have RMS (90.7%, 89.8%), the remainder have PPMS (9.3%, 10.2%). The mean (SD) duration since MS symptom onset was 7.7 (8.3) and 6.8 years (7.1), and mean (SD) duration since MS diagnosis was 5.7 (6.8) and 4.8 years (5.8) years in patients receiving OCR SC and IV, respectively. At BL, the mean (SD) number of Gd+T1 lesions was 0.5 (1.7) in OCR SC and 1.0 (2.5) in OCR IV treated patients. The BL data of patients enrolled in OCARINA II reflect a typical MS population for which OCR IV is currently indicated. The new route of administration has the potential to deliver the clinical benefits of OCR while providing treatment flexibility along with an additional treatment choice.

The Phase III OCARINA II trial evaluating OCREVUS® (ocrelizumab) as a twice a year 10-minute subcutaneous injection met its primary and secondary endpoints (or objectives). Ocrelizumab subcutaneous injection was shown to be non-inferior to ocrelizumab given by intravenous infusion (IV), as measured by pharmacokinetics (levels in the blood) over 12 weeks. Ocrelizumab subcutaneous injection was also comparable with ocrelizumab IV in controlling magnetic resonance imaging (MRI) lesion activity in the brain over 12 weeks. Ocrelizumab subcutaneous injection led to a rapid, complete and sustained B-cell depletion, similar to ocrelizumab given by intravenous infusion. The safety profile of ocrelizumab subcutaneous injection was consistent with that of ocrelizumab given by IV. No new safety signals were identified. No treatment-emergent anti-drug antibodies (ADA) were observed. Secondary objectives included Cmax, MRI lesions in controlled period (e.g., the first 24 weeks of the treatment), B-cell depletion, and immune response to ocrelizumab subcutaneous and IV formulations and rHuPH20.

The ocrelizumab 10-minute injection is designed to be administered without the need for IV infrastructure so it facilitates the usage of ocrelizumab in MS centers without IV infrastructure or those with IV capacity limitations. It also retains the twice-yearly dosing regimen of ocrelizumab IV that has shown high persistence and adherence since becoming a standard of care MS treatment. This provides an additional delivery option so that the administration of ocrelizumab can be matched to the individual needs of patients and healthcare professionals.

The investigational subcutaneous formulation combines ocrelizumab with Halozyme Therapeutics' Enhanze® drug delivery technology.

### Results:

### A. Pharmacokinetics

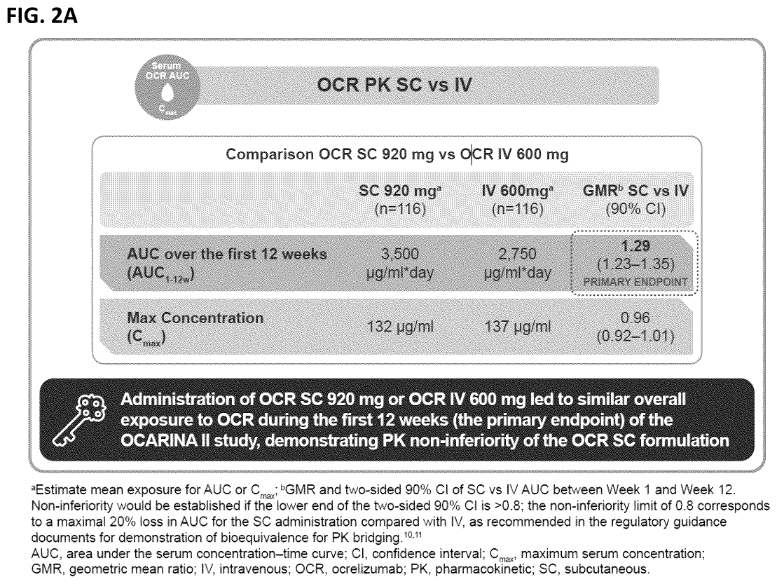

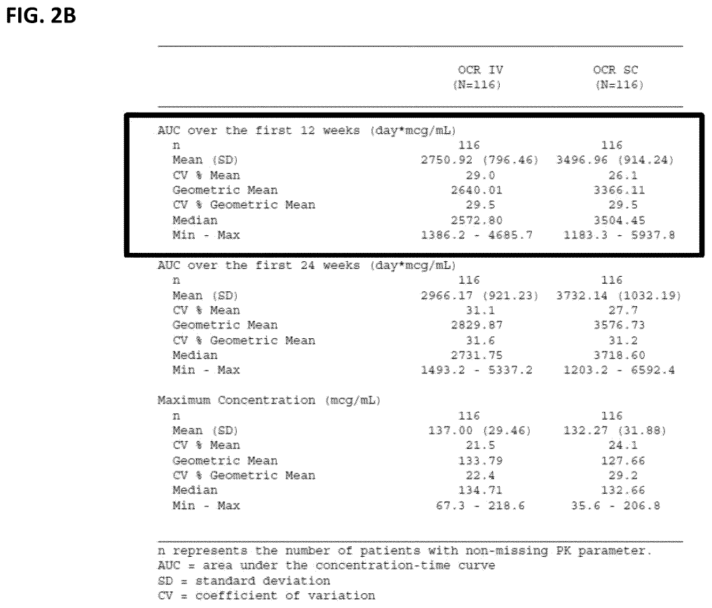

As shown in FIGS. 2A-2B, the primary objective (pharmacokinetics) was met. Administration of OCR SC 920 mg and OCR IV 600 mg led to similar overall exposure to OCR during the first 12 weeks (the primary endpoint) of the OCARINA II study.

### B. Radiological Objectives (MRI)

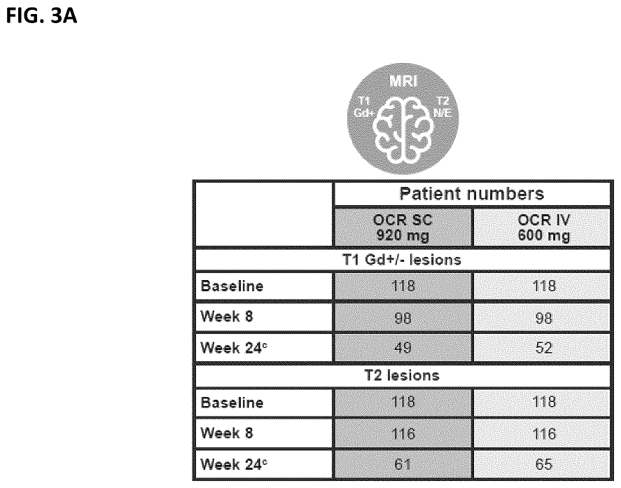

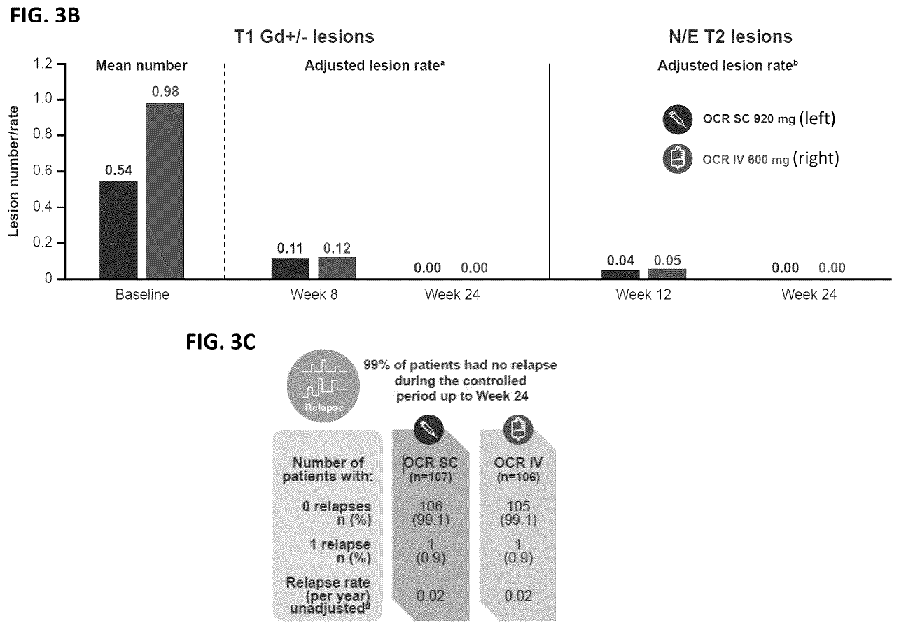

As shown in FIGS. 3A-3C, secondary radiological objectives were met. Ocrelizumab (OCR) SC is similar to ocrelizumab IV in controlling MRI lesion activity in the brain in the controlled period. Out of 107 Ocrelizumab SC patients, 106 patients had no relapses in the first 24 weeks. Out of 106 Ocrelizumab IV patients, 105 patients had no relapses in the first 24 weeks. In conclusion: OCR SC 920 mg administration resulted in near-complete suppression of MRI lesion activity, and most patients had no relapse up to week 24.

### C. Pharmacodynamics

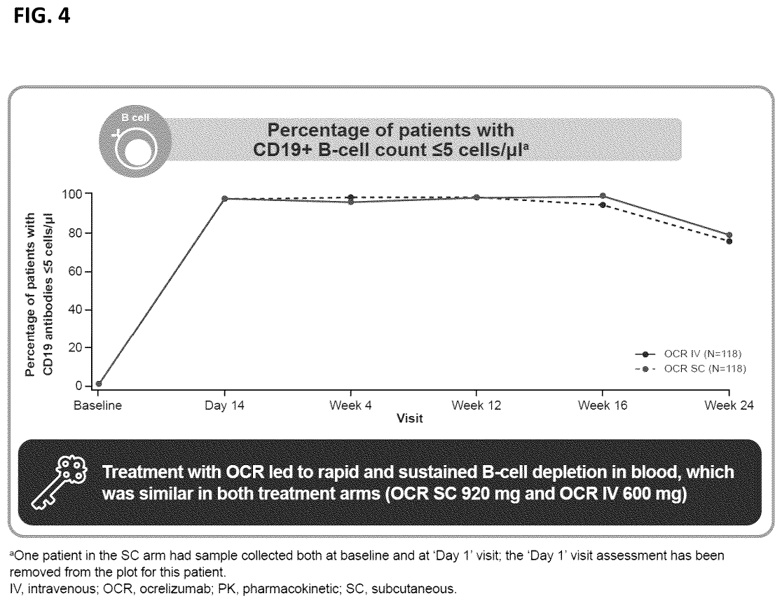

As shown in FIG. 4, ocrelizumab SC led to a rapid, complete and sustained B-cell depletion. Baseline: the last assessment before the first exposure.

### D. Safety

Safety profile of ocrelizumab SC is similar to safety profile of ocrelizumab IV. No new safety signals identified from known risks associated to ocrelizumab and route of administration.

### E. Immunogenicity

No treatment-emergent ADA observed in both treatment arms in the patients which already completed the 24-week controlled period (63/118 patients in both arms). No treatment emergent ocrelizumab Anti-Drug Antibodies (ADAs) were observed in both groups (IV and SC). No treatment emergent antibodies against anti-rHuPH20 antibodies in both groups (IV and SC).

### Summary and Conclusion

These data suggest that ocrelizumab SC delivers similar clinical benefits as ocrelizumab IV, while providing treatment flexibility along with an additional treatment choice for patients and healthcare providers.

The study achieved its primary objective of demonstrating non-inferiority of ocrelizumab SC 920 mg to ocrelizumab IV 600 mg with respect to AUCW₁₋₁₂.

The selected ocrelizumab SC 920 mg dose was well-tolerated, and achieved similar levels of B-cell depletion as seen with ocrelizumab IV 600 mg, with no treatment emergent ADAs to ocrelizumab.

Administration of ocrelizumab SC 920 mg resulted in near-complete suppression of radiological (MRI) and clinical (relapses) disease activity as measured up to Week 24, similarly to ocrelizumab IV 600 mg.

The safety profile of IRs was consistent with the characteristics identified in OCARINA I and did not limit treatment.

V. SEQUENCE LISTING

| SEQ | | |
|---|---|---|
| ID | | |
| NO: | Sequence | Reference |
| 1 | RASSSVSYMH | CDR L1 sequence in ocrelizumab |
| 2 | APSNLAS | CDR L2 sequence in ocrelizumab |
| 3 | QQWSFNPPT | CDR L3 sequence in ocrelizumab |
| 4 | GYTFTSYNMH | CDR H1 sequence in ocrelizumab |
| 5 | AIYPGNGDTSYNQKFKG | CDR H2 sequence in ocrelizumab |
| 6 | VVYYSNSYWYFDV | CDR H3 sequence in ocrelizumab |
| 7 | DIQMTQSPSSLSASVGDRVTITCRASSSVSYM | V_{L} sequence in ocrelizumab |
| | HWYQQKPGKAPKPLIYAPSNLASGVPSRFSG | |
| | SGSGTDFTLTISSLQPEDFATYYCQQWSFNPP | |
| | TFGQGTKVEIKR | |
| 8 | EVQLVESGGGLVQPGGSLRLSCAASGYTFTS | V_{H} sequence in ocrelizumab |
| | YNMHWVRQAPGKGLEWVGAIYPGNGDTSY | |
| | NQKFKGRFTISVDKSKNTLYLQMNSLRAEDT | |
| | AVYYCARVVYYSNSYWYFDVWGQGTLVTV | |
| | SS | |
| 9 | DIQMTQSPSSLSASVGDRVTITCRASSSVSYM | Light chain sequence in |
| | HWYQQKPGKAPKPLIYAPSNLASGVPSRFSG | ocrelizumab |
| | SGSGTDFTLTISSLQPEDFATYYCQQWSFNPP | |
| | TFGQGTKVEIKRTVAAPSVFIFPPSDEQLKSG | |
| | TASVVCLLNNFYPREAKVQWKVDNALQSG | |
| | NSQESVTEQDSKDSTYSLSSTLTLSKADYEK | |
| | HKVYACEVTHQGLSSPVTKSFNRGEC | |
| 10 | EVQLVESGGGLVQPGGSLRLSCAASGYTFTS | Heavy chain sequence in |
| | YNMHWVRQAPGKGLEWVGAIYPGNGDTSY | ocrelizumab with terminal lysine |
| | NQKFKGRFTISVDKSKNTLYLQMNSLRAEDT | |
| | AVYYCARVVYYSNSYWYFDVWGQGTLVTV | |
| | SSASTKGPSVFPLAPSSKSTSGGTAALGCLVK | |
| | DYFPEPVTVSWNSGALTSGVHTFPAVLQSSG | |
| | LYSLSSVVTVPSSSLGTQTYICNVNHKPSNTK | |
| | VDKKVEPKSCDKTHTCPPCPAPELLGGPSVF | |
| | LFPPKPKDTLMISRTPEVTCVVVDVSHEDPE | |
| | VKFNWYVDGVEVHNAKTKPREEQYNSTYR | |
| | VVSVLTVLHQDWLNGKEYKCKVSNKALPA | |
| | PIEKTISKAKGQPREPQVYTLPPSREEMTKNQ | |
| | VSLTCLVKGFYPSDIAVEWESNGQPENNYKT | |
| | TPPVLDSDGSFFLYSKLTVDKSRWQQGNVFS | |
| | CSVMHEALHNHYTQKSLSLSPGK | |
| 11 | EVQLVESGGGLVQPGGSLRLSCAASGYTFTS | Heavy chain sequence in |
| | YNMHWVRQAPGKGLEWVGAIYPGNGDTSY | ocrelizumab without terminal |
| | NQKFKGRFTISVDKSKNTLYLQMNSLRAEDT | lysine |
| | AVYYCARVVYYSNSYWYFDVWGQGTLVTV | |
| | SSASTKGPSVFPLAPSSKSTSGGTAALGCLVK | |
| | DYFPEPVTVSWNSGALTSGVHTFPAVLQSSG | |
| | LYSLSSVVTVPSSSLGTQTYICNVNHKPSNTK | |
| | VDKKVEPKSCDKTHTCPPCPAPELLGGPSVF | |
| | LFPPKPKDTLMISRTPEVTCVVVDVSHEDPE | |
| | VKFNWYVDGVEVHNAKTKPREEQYNSTYR | |
| | VVSVLTVLHQDWLNGKEYKCKVSNKALPA | |
| | PIEKTISKAKGQPREPQVYTLPPSREEMTKNQ | |
| | VSLTCLVKGFYPSDIAVEWESNGQPENNYKT | |
| | TPPVLDSDGSFFLYSKLTVDKSRWQQGNVFS | |
| | CSVMHEALHNHYTQKSLSLSPG | |

1. A method of treating multiple sclerosis in a patient comprising subcutaneously administering an anti-CD20 antibody to the patient at a dose of 920 mg, wherein the anti-CD20 antibody is ocrelizumab.

2. The method of claim 1, wherein the patient has not been subjected to a prior intravenous administration of the anti-CD20 antibody at a dose of about 600 mg.

3. The method of claim 1, wherein the patient has not been subjected to a prior intravenous administration of the anti-CD20 antibody.

4. The method of claim 1, wherein the patient has not been subjected to a prior subcutaneous administration of the anti-CD20 antibody at a dose lower than about 900 mg.

5. The method of claim 1, wherein the patient has received a prior treatment with an anti-CD20 antibody.

6. The method of claim 5, wherein the anti-CD20 antibody is ocrelizumab.

7. The method of claim 1, wherein the method comprises subcutaneously administering more than one dose of the anti-CD20 antibody, wherein each dose comprises 920 mg of the anti-CD20 antibody.

8. The method of claim 7, wherein the anti-CD20 antibody is administered at a frequency of once every 6 months.

9. The method of claim 1, wherein the anti-CD20 antibody is the only medicament administered to the patient to treat multiple sclerosis.

10. The method of claim 1, wherein the anti-CD20 antibody is in a pharmaceutical formulation, wherein the pharmaceutical formulation comprises:
a) 40 mg/ml anti-CD20 antibody;
b) 190-290 mM trehalose;
c) 5-15 mM methionine;
d) 0.04-0.08% (w/v) polysorbate 20; and
e) 1000 U/ml hyaluronidase;
wherein the pharmaceutical formulation has a pH of 5.0-5.6.

11. The method of claim 10, wherein the pharmaceutical formulation comprises 15-25 mM sodium acetate.

12. The method of claim 10, wherein the hyaluronidase comprises a recombinant human hyaluronidase.

13. The method of claim 12, wherein the recombinant human hyaluronidase is rHuPH20.

14. The method of claim 11, wherein the hyaluronidase comprises a recombinant human hyaluronidase.

15. The method of claim 14, wherein the hyaluronidase is rHuPH20.

16. The method of claim 1, wherein the patient has been subject to oral dexamethasone and antihistamine prior to the subcutaneous administration of the anti-CD20 antibody.

17. The method of claim 16, wherein the amount of dexamethasone is 20 mg.

18. The method of claim 1, wherein the multiple sclerosis is relapsing multiple sclerosis (RMS).

19. The method of claim 18, wherein the multiple sclerosis is clinical isolated syndrome (CIS), relapsing-remitting multiple sclerosis (RRMS) or active secondary progressive multiple sclerosis (SPMS).

20. The method of claim 1, wherein the multiple sclerosis is a primary progressive multiple sclerosis (PPMS).

21. The method of claim 1, wherein the anti-CD20 antibody is comprised in a pharmaceutical formulation and the subcutaneous administration takes no more than about 10 minutes.

22. The method of claim 21, wherein the subcutaneous administration takes about 10 minutes.

23. The method of claim 1, wherein the anti-CD20 antibody is in a pharmaceutical formulation, wherein the pharmaceutical formulation comprises 40 mg/ml anti-CD20 antibody.

24. The method of claim 23, wherein the subcutaneous administration takes no more than about 10 minutes.

25. The method of claim 24, wherein the subcutaneous administration takes about 10 minutes.


SEQUENCE LISTING (11 sequences)

SEQ ID NO: 1 - protein, 10 aa, synthetic construct
RASSSVSYMH

SEQ ID NO: 2 - protein, 7 aa, synthetic construct
APSNLAS

SEQ ID NO: 3 - protein, 9 aa, synthetic construct
QQWSFNPPT

SEQ ID NO: 4 - protein, 10 aa, synthetic construct
GYTFTSYNMH

SEQ ID NO: 5 - protein, 17 aa, synthetic construct
AIYPGNGDTSYNQKFKG

SEQ ID NO: 6 - protein, 13 aa, synthetic construct
VVYYSNSYWYFDV

SEQ ID NO: 7 - protein, 107 aa, synthetic construct
DIQMTQSPSSLSASVGDRVTITCRASSSVSYMHWYQQKPGKAPKPLIYAPSNLASGVPSRFSGSGSGTDFTLTISSLQPEDFATYYCQQWSFNPPTFGQGTKVEIKR

SEQ ID NO: 8 - protein, 122 aa, synthetic construct [sequence omitted]

SEQ ID NO: 9 - protein, 213 aa, synthetic construct
DIQMTQSPSSLSASVGDRVTITCRASSSVSYMHWYQQKPGKAPKPLIYAPSNLASGVPSRFSGSGSGTDFTLTISSLQPEDFATYYCQQWSFNPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 10 - protein, 452 aa, synthetic construct
EVQLVESGGGLVQPGGSLRLSCAASGYTFTSYNMHWVRQAPGKGLEWVGAIYPGNGDTSYNQKFKGRFTISVDKSKNTLYLQMNSLRAEDTAVYYCARVVYYSNSYWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 11 - protein, 451 aa, synthetic construct
EVQLVESGGGLVQPGGSLRLSCAASGYTFTSYNMHWVRQAPGKGLEWVGAIYPGNGDTSYNQKFKGRFTISVDKSKNTLYLQMNSLRAEDTAVYYCARVVYYSNSYWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG